(19) United States
(12) Patent Application Publication
(10) Pub. No.: US 2024/0360470 A1
(43) Pub. Date: Oct. 31, 2024

(54) COMPOSITIONS AND METHODS OF MODIFYING A PLANT GENOME TO PRODUCE A MS1 OR MS5 MALE-STERILE PLANT

(71) Applicant: PIONEER HI-BRED INTERNATIONAL, INC., JOHNSTON, IA (US)

(72) Inventors: UTE BAUMANN, GLEN OSMOND (AU); ANDREW MARK CIGAN, MADISON, WI (US); MARGARET ANNE PALLOTTA, NORWOOD (AU); MANJIT SINGH, JOHNSTON, IA (US); RADOSLAW SUCHECKI, PASADENA (AU); NATHAN SPENCER WATSON-HAIGH, NOVAR GARDENS (AU); RYAN WHITFORD, EASTWOOD (AU)

(73) Assignee: PIONEER HI-BRED INTERNATIONAL, INC., JOHNSTON, IA (US)

(21) Appl. No.: 18/765,988

(22) Filed: Jul. 8, 2024

Related U.S. Application Data
(63) Continuation of application No. 16/769,019, filed on Jun. 2, 2020, now Pat. No. 12,054,732.
(60) Provisional application No. 62/597,002, filed on Dec. 11, 2017.

(51) Int. Cl. C12N 15/82; C07K 14/415

(52) U.S. Cl. CPC C12N 15/8289; C07K 14/415; C12N 15/8213

(57) ABSTRACT

Compositions and methods are provided for genome modification of a nucleotide sequence located in or near a male fertility gene of Ms1 or Ms5 in the genome of a plant cell or plant to produce a male-sterile plant. In some examples, the methods and compositions employ a guide RNA/Cas endonuclease system for modifying or altering target sites located in or near a male fertility gene of Ms1 or Ms5 in the genome of a plant cell, plant or seed to produce a male-sterile plant. Also provided are compositions and methods employing a guide polynucleotide/Cas endonuclease system for genome modification a nucleotide sequence located in or near a male fertility gene of Ms1 or Ms5 in the genome of a plant cell to produce a male-sterile plant. Compositions and methods are also provided for restoring fertility to a Ms1 or Ms5 nucleotide sequence to a male-sterile Ms1 or Ms5 plant.

### CROSS REFERENCE

This patent application claims the benefit of and priority to U.S. Provisional Application No. 62/597,002, filed Dec. 11, 2017, which is incorporated herein by reference in its entirety.

### FIELD

The disclosure relates to the field of plant molecular biology, in particular, to compositions and methods of modifying a plant's genome to alter the male-fertility of a plant.

### REFERENCE TO SEQUENCE LISTING SUBMITTED ELECTRONICALLY

The official copy of the sequence listing is submitted electronically via EFS-Web as an ASCII formatted sequence listing with a file named RTS20250GWOPCT_SeqLstg_ST25.txt, produced on Dec. 10, 2018, and having a size 152 kilobytes and is filed concurrently with the specification. The sequence listing contained in this ASCII formatted document is part of the specification and is herein incorporated by reference in its entirety.

### BACKGROUND

Development of hybrid plant breeding has made possible considerable advances in quality and quantity of crops produced. Increased yield and combination of desirable characteristics, such as resistance to disease and insects, heat and drought tolerance, along with variations in plant composition are all possible because of hybridization procedures. These procedures frequently rely heavily on providing for a male parent contributing pollen to a female parent to produce the resulting hybrid.

Field crops are bred through techniques that take advantage of the plant's method of pollination. A plant is self-pollinating if pollen from one flower is transferred to the same or another flower of the same plant or a genetically identical plant. A plant is cross-pollinated if the pollen comes from a flower on a different plant.

In certain species, such as Brassica campestris, the plant is normally self-sterile and can only be cross-pollinated. In self-pollinating species, such as soybeans, cotton and wheat, the male and female plants are anatomically juxtaposed. During natural pollination, the male reproductive organs of a given flower pollinate the female reproductive organs of the same flower. Maize has male flowers, located on the tassel, and female flowers, located on the ear, on the same plant and can be bred by both self-pollination and cross-pollination techniques,

The development of hybrids requires the crossing of homozygous inbred parents. A hybrid variety is the cross of two such inbred lines, each of which may have one or more desirable characteristics lacked by the other or which complement the other. The new inbreds are crossed with other inbred lines and the hybrids from these crosses are evaluated to determine which have commercial potential. The hybrid progeny of the first generation is designated F₁. In the development of hybrids only the F1 hybrid plants are sought. The F1 hybrid is more vigorous than its inbred parents. This hybrid vigor, or heterosis, can be manifested in many ways, including increased vegetative growth and increased yield.

During hybrid seed production, it is desirable to prevent self-pollination of the female inbred to avoid production and harvesting of female inbred seeds, since they exhibit less vigor than the hybrid seeds. To increase commercial quantities of the resulting hybrid seed, hybrid seed is often obtained using male-sterile female parents. Manual emasculation of the female can be labor intensive and/or impractical, depending on the crop. For example, in wheat, both male flowers and female flowers are located within the same floret on a spike making it challenging to prevent self-pollination. As a result, male-sterile female plants created from either chemical or genetic manipulations are often used in hybrid seed production.

### SUMMARY

Provided herein are methods for producing male-sterile plants. In one embodiment, the method includes introducing a genetic modification into at least one or more endogenous MS1 or MS5 polynucleotide sequences in a plant cell, wherein the genetic modification confers male sterility to a plant obtained from the plant cell. In one aspect, the genetic modification is introduced using biotechnology approaches. Accordingly, also provided herein are male-sterile plants that contain a genetic modification in at least one or more endogenous MS1 or MS5 polynucleotide sequences. The genetic modification may confer male sterility to a plant obtained from the plant cell.

In yet another aspect, the method includes providing to a plant cell a guide RNA and a Cas endonuclease. The RNA and Cas endonuclease are capable of forming a complex that enables the Cas endonuclease to introduce a double strand break at a target site located in or near a male fertility gene of MS1 or MS5. The method may additionally include identifying at least one plant cell that has the modification. The modification may be at least one deletion, insertion, or substitution of one or more nucleotides in a MS1 or MS5 gene that confers male-sterility to a plant. A male-sterile plant may be obtained from the plant cell.

A male-sterile plant may have at least one altered target site that confers male-sterility to the plant. The target site may originate from a corresponding target site that was recognized and cleaved by a guideRNA/Cas endonuclease system. The target site may be located in or near a male fertility gene of MS1 or MS5 and affect the expression level of the MS1 or MS5 gene so that the plant is male-sterile.

Also provided herein is a method for producing a male sterile plant that includes obtaining or providing a first plant comprising at least one Cas endonuclease capable of introducing a double strand break at a genomic target site located in a male fertility gene locus of MS1 or MS5 in the plant genome and a second plant comprising a guide RNA that is capable of forming a complex with the Cas endonuclease. In some aspects, the first and second plants may be crossed and the progeny evaluated for those that have an altered target site. Male-sterile progeny plants may be selected. Accordingly, also included herein are male-sterile progeny plants produced by any of the methods disclosed herein. The progeny plant may include at least one altered target site that originated from a corresponding target site that was recognized and cleaved by a guideRNA/Cas endonuclease system. The altered target site may be located in or near a male fertility gene of MS1 or MS5 and affect the expression level of the MS1 or MS5 gene so that the plant is male-sterile.

A method of modifying the male-fertility of a plant that includes introducing at least one guide RNA, at least one polynucleotide modification template and at least one Cas endonuclease into a plant cell is provided herein. The Cas endonuclease may introduce a double-strand break at a target site located in or near a MS1 or MS5 gene in the genome of the plant cell. The polynucleotide modification template includes at least one nucleotide modification of a nucleotide sequence at the target site, and the modification modifies the expression level of the MS1 or MS5 gene. A male-sterile plant may be obtained from the plant cell.

Also provided herein are methods for restoring male fertility in a male-sterile plant. A male sterile plant produced by any of the methods disclosed herein and having one or more endogenous MS1 or MS5 genes with a genetic modification that confers male-sterility to the plant may have fertility restored by introducing one or more polynucleotide sequences that encode a MS1 or MS5 polypeptide.

Also provided herein are isolated nucleic acids that impact male fertility of a plant. In some aspects, an isolated nucleic acid that impacts male fertility of a plant is a polynucleotide sequence of: (a) a polynucleotide comprising the sequence set forth in SEQ ID NO: 16, 18, 21, 23-24, 28-29, 31-32, 199, 36, 38, 41, 43, 46, 48, 51 or 53; (b) a polynucleotide having at least 85%, 90% or 95% sequence identity to SEQ ID NO: 16, 18, 21, 23-24, 28-29, 31-32, 199, 36, 38, 41, 43, 46, 48, 51 or 53; (c) a polynucleotide that encodes a polypeptide having at least 85%, 90% or 95% sequence identity to SEQ ID NO: 19, 25-26, 33-34, 39, 44, 49, or 54; (d) a polynucleotide that encodes a polypeptide of SEQ ID NO: 19, 25-26, 33-34, 39, 44, 49, or 54; (e) a polynucleotide sequence which hybridizes to the full length of SEQ ID NO: 16, 18, 21, 23-24, 28-29, 31-32, 199, 36, 38, 41, 43, 46, 48, 51 or 53 under highly stringent conditions of a wash of 0.1 SSC, 0.1% (w/v) SDS at 65 degrees Celsius. In some aspects, the nucleic acid is in an expression vector.

Also provided herein is an isolated polypeptide that impacts the male fertility of a plant. In some aspects, the isolated polypeptide that impacts male fertility of a plant is an amino acid sequence of: (a) an amino acid sequence that has at least 85%, 90% or 95% sequence identity to the amino acid sequence set forth in SEQ ID NO: 19, 25-26, 33-34, 39, 44, 49, or 54, wherein said polypeptide impacts the male fertility of the plant; (b) an amino acid sequence comprising the amino acid sequence set forth in SEQ ID NO: 19, 25-26, 33-34, 39, 44, 49, or 54; (c) an amino acid sequence comprising at least 100 contiguous amino acids of the amino acid sequence set forth in SEQ ID NO: 19, 25-26, 33-34, 39, 44, 49, or 54; (d) an amino acid sequence encoded by a polynucleotide that has at least 85%, 90% or 95% sequence identity to SEQ ID NO: 16, 18, 21, 23-24, 28-29, 31-32, 199, 36, 38, 41, 43, 46, 48, 51 or 53; and (e) an amino acid sequence encoded by a polynucleotide of SEQ ID NO: 16, 18, 21, 23-24, 28-29, 31-32, 199, 36, 38, 41, 43, 46, 48, 51 or 53; and (f) a polynucleotide sequence which hybridizes to the full length of SEQ ID NO: 16, 18, 21, 23-24, 28-29, 31-32, 199, 36, 38, 41, 43, 46, 48, 51 or 53, or 55 under highly stringent conditions of a wash of 0.1 SSC, 0.1% (w/v) SDS at 65 degrees Celsius. Also provided herein are plant cells or plants having the nucleic acid and/or expressing the polypeptide.

In another aspect, disclosed herein is an isolated regulatory region driving male-tissue-preferred or specific expression that includes the sequence of SEQ ID NO: 17, 22, 30, 37, 42, 47, 52, or 200 and functional fragments thereof. Also disclosed herein are plant cells comprising the regulatory region. The regulatory region may be operably linked to a heterologous coding sequence. In some aspects, the regulatory region is included in a DNA construct to drive expression of a sequence of interest, for example, a heterologous polynucleotide. The regulatory region may be used to express a polynucleotide of interest in male tissue of a plant. In one aspect, the method includes introducing into the plant a polynucleotide having a polynucleotide sequence of SEQ ID NO: 17, 22, 30, 37, 42, 47, 52, or 200, and functional fragments thereof. The polynucleotide sequence may confer male-tissue-specific or preferred expression of an operably linked sequence.

### BRIEF DESCRIPTION OF THE FIGURES

FIG. 1 shows an alignment of barley (SEQ ID NO:39) and wheat (SEQ ID NO: 19) Ms5 amino acid sequences.

FIG. 2 is an alignment of MS5 homologues of Hordeum vulgare (SEQ ID NO: 39), Triticum aestivum (SEQ ID NO:19), Brachypodium distachyon (SEQ ID NO: 44) and Oryza sativa (SEQ ID NO:49).

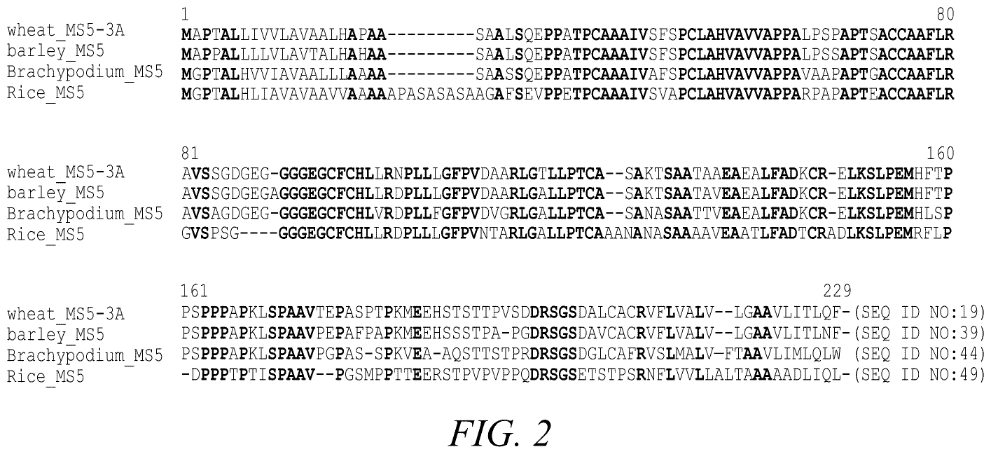

### DETAILED DESCRIPTION

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

Mutations that cause male sterility in plants have the potential to be useful in methods for hybrid seed production. For example, use of a male-sterile female inbred plant as a parent to produce hybrid seed can lower production costs by eliminating the need for the labor-intensive removal of male flowers and self-pollination of the female inbred. Emasculation of wheat can be especially challenging since the male flowers and female flowers are located within the same floret. This makes it difficult to prevent self-pollination of the female and fertilize it with pollen from another wheat plant. Self-pollination results in seed of the female inbred being harvested along with the hybrid seed which is normally produced. Female inbred seed does not exhibit heterosis and therefore is not as commercially desirable as F₁ seed. Thus, use of a male-sterile female inbred prevents self-fertilization while maintaining the purity of hybrid seeds.

Mutations that cause male sterility in crop plants such as maize, wheat and rice have been produced by a variety of methods such as X-rays or UV-irradiations, chemical treatments, or transposable element insertions (ms23, ms25, ms26, ms32) (Chaubal et al. 2000) Am J Bot 87:1193-1201). However, such methods are random mutagenesis methods that induce mutations randomly throughout the genome and not just in the gene of interest. Typically, with such random mutagenesis methods, it requires considerable effort to identify a plant that contains a mutation in the gene of interest and it is by no means certain that such a plant will be identified. Furthermore, with random mutagenesis methods, each plant tested is likely to carry multiple mutations. Therefore, a plant that is identified with the mutation in the gene of interest must be backcrossed for several or more generations to eliminate the undesired mutations.

In contrast to such random mutagenesis methods, the described herein are methods for producing male sterile plants by introducing a genetic modification into at least one or more endogenous fertility genes, such as MS1 or MS5 polynucleotide sequences, in a plant cell. The introduced genetic modification confers male sterility to a plant arising from the plant cell. Preferably the plant is a crop plant.

PCT Patent publication WO2016048891A1, published Mar. 31, 2016, describes a male fertility gene referred to as “MS1” that is located on wheat chromosome 4BS and encodes a glycosylphosphatidylinositol (GPI)-anchored nsLTP (LTPG) polypeptide (referred to as TaLTPGI) important to male fertility. Examples of DNA and polypeptide sequences of barley, wheat, rice, and Brachypodium Ms1 are disclosed in WO2016048891A1, published Mar. 31, 2016.

A mutated gene in FS20 referred to as ms5 was mapped to the long arm on wheat chromosome 3A. See Klindworth et al. “Chromosomal Location of Genetic Male Sterility Genes in Four Mutants of Hexaploid Wheat” Crop Science (2002) 42:1447-1450.

Additionally, the present disclosure includes the following MS1 and MS5 polynucleotides and polypeptides:

TABLE 1
Summary of SEQ ID NOS:

| SEQ ID NO: | Description |
|---|---|
| 1 | Wheat Ms1 A genomic (exon-intron) |
| 2 | Wheat Ms1 A promoter |
| 3 | Wheat Ms1 A coding |
| 4 | Wheat Ms1 A amino acid |
| 5 | Wheat Ms1 A terminator |
| 6 | Wheat Ms1 B genomic (exon-intron) |
| 7 | Wheat Ms1 B promoter |
| 8 | Wheat Ms1 B coding |
| 9 | Wheat Ms1 B amino acid |
| 10 | Wheat Ms1 B terminator |
| 11 | Wheat Ms1 D genomic (exon-intron) |
| 12 | Wheat Ms1 D promoter |
| 13 | Wheat Ms1 D coding |
| 14 | Wheat Ms1 D amino acid |
| 15 | Wheat Ms1 D terminator |
| 16 | Wheat Ms5 3A genomic (exon-intron) |
| 17 | Wheat Ms5 3A promoter |
| 18 | Wheat Ms5 3A coding |
| 19 | Wheat Ms5 3A amino acid |
| 20 | Wheat Ms5 3A terminator |
| 21 | Wheat Ms5 3B genomic (exon-intron) |
| 22 | Wheat Ms5 3B promoter |
| 23 | Wheat Ms5 3B coding |
| 24 | Wheat Ms5 3B coding |
| 25 | Wheat Ms5 3B amino acid |
| 26 | Wheat Ms5 3B amino acid |
| 27 | Wheat Ms5 3B terminator |
| 28 | Wheat Ms5 3D genomic (exon-intron) |
| 29 | Wheat Ms5 3D genomic (exon-intron) |
| 30 | Wheat Ms5 3D promoter |
| 31 | Wheat Ms5 3D coding |
| 32 | Wheat Ms5 3D coding |
| 33 | Wheat Ms5 3D amino acid |
| 34 | Wheat Ms5 3D amino acid |
| 35 | Wheat Ms5 3D terminator |
| 36 | Barley MS5 genomic (exon-intron) |
| 37 | Barley Ms5 promoter |
| 38 | Barley Ms5 coding |
| 39 | Barley Ms5 amino acid |
| 40 | Barley Ms5 terminator |
| 41 | Brachypodium distachyon Ms5 |
| | genomic (exon-intron) |
| 42 | Brachypodium distachyon Ms5 |
| | promoter |
| 43 | Brachypodium distachyon Ms5 coding |
| 44 | Brachypodium distachyon Ms5 amino |
| | acid |
| 45 | Brachypodium distachyon Ms5 |
| | terminator |
| 46 | Rice Ms5 genomic (exon-intron) |
| 47 | Rice Ms5 promoter |
| 48 | Rice Ms5 coding |
| 49 | Rice Ms5 amino acid |
| 50 | Rice Ms5 terminator |
| 51 | Maize Ms5 genomic (exon-intron) |
| 52 | Maize Ms5 promoter |
| 53 | Maize Ms5 coding |
| 54 | Maize Ms5 amino acid |
| 55 | Maize Ms5 terminator |
| 56 | Wheat Ms1 B CR1 target |
| 57 | Wheat Ms1 B CR2 target |
| 58 | Wheat Ms1 B CR3 target |
| 59 | Wheat Ms1 B CR4 target |
| 60 | Wheat Ms1 B CR5 target |
| 61 | Wheat Ms1 B CR6 target |
| 62 | Wheat Ms1 B CR7 target |
| 63 | Wheat Ms1 B CR8 target |
| 64 | Wheat Ms1 B CR9 target |
| 65 | Wheat Ms1 B CR10 target |
| 66 | Wheat Ms1 B CR11 target |
| 67 | Wheat Ms5 CR1 target |
| 68 | Wheat Ms5 CR2 target |
| 69 | Wheat Ms5 CR3 target |
| 70 | Wheat Ms5 CR4 target |
| 71 | Wheat Ms5 CR5 target |
| 72 | Wheat Ms5 CR6 target |
| 73 | Wheat Ms5 CR7 target |
| 74 | Wheat Ms5 CR8 target |
| 75 | Wheat Ms5 CR9 target |
| 76 | Wheat Ms5 CR10 target |
| 77 | Wheat Ms5 CR11 target |
| 78 | Wheat Ms5 CR12 target |
| 79 | Wheat Ms5 CR13 target |
| 80 | Wheat Ms5 CR14 target |
| 81 | Wheat Ms5 CR15 target |
| 82 | Wheat Ms1 CR1 guide |
| 83 | Wheat Ms1 CR2 guide |
| 84 | Wheat Ms1 CR3 guide |
| 85 | Wheat Ms1 CR4 guide |
| 86 | Wheat Ms1 CR5 guide |
| 87 | Wheat Ms1 CR6 guide |
| 88 | Wheat Ms1 CR7 guide |
| 89 | Wheat Ms1 CR8 guide |
| 90 | Wheat Ms1 CR9 guide |
| 91 | Wheat Ms1 CR10 guide |
| 92 | Wheat Ms1 CR11 guide |
| 93 | Wheat Ms5 CR1 guide |
| 94 | Wheat Ms5 CR2 guide |
| 95 | Wheat Ms5 CR3 guide |
| 96 | Wheat Ms5 CR4 guide |
| 97 | Wheat Ms5 CR5 guide |
| 98 | Wheat Ms5 CR6 guide |
| 99 | Wheat Ms5 CR7 guide |
| 100 | Wheat Ms5 CR8 guide |
| 101 | Wheat Ms5 CR9 guide |
| 102 | Wheat Ms5 CR10 guide |
| 103 | Wheat Ms5 CR11 guide |
| 104 | Wheat Ms5 CR12 guide |
| 105 | Wheat Ms5 CR13 guide |
| 106 | Wheat Ms5 CR14 guide |
| 107 | Wheat Ms5 CR15 guide |
| 108 | Genome edit insertion of one |
| | nucleotide(A) at position 18 of wheat |
| | MS1 B coding sequence |
| 109 | Genome edit insertion of one |
| | nucleotide(T) at position 18 of wheat |
| | MS1 B coding sequence |
| 110 | Genome edit insertion of one |
| | nucleotide(C) at position 18 of wheat |
| | MS1 B coding sequence |
| 111 | Genome edit deletion of three |
| | nucleotides at position 18 of wheat |
| | MS1 B coding sequence |
| 112 | Wheat 3A marker MP0061 amplicon |
| 113 | Wheat 3A marker MP0070 amplicon |
| 114 | Wheat 3A marker MP0079 amplicon |
| 115 | Wheat 3A marker MP0090 amplicon |
| 116 | Wheat 3A marker MP0091 amplicon |
| 117 | Wheat 3A marker MP0156 amplicon |
| 118 | Wheat 3A marker MP0179 amplicon |
| 119 | Wheat 3A marker MP0182 amplicon |
| 120 | Wheat 3A marker MP0190 amplicon |
| 121 | Wheat 3A marker MP0191 amplicon |
| 122 | Wheat 3A marker MP0192 amplicon |
| 123 | Wheat 3A marker MP0201 amplicon |
| 124 | Wheat 3D marker MP0126 amplicon |
| 125 | Wheat 3D marker MP0127 amplicon |
| 126 | Wheat 3D marker MP0130 amplicon |
| 127 | Wheat 3D marker MP0131 amplicon |
| 128 | Wheat 3D marker MP0211 amplicon |
| 129 | Wheat 3D marker MP0212 amplicon |
| 130 | Wheat 3D marker MP0215 amplicon |
| 131 | Wheat 3D marker MP0216 amplicon |
| 132 | Wheat Ms5 3A qRT-PCR primer |
| | Forward |
| 133 | Wheat Ms5 3A qRT-PCR primer |
| | Reverse |
| 134 | Wheat Ms5 3B qRT-PCR primer |
| | Forward |
| 135 | Wheat Ms5 3B qRT-PCR primer |
| | Reverse |
| 136 | Wheat Ms5 3D qRT-PCR primer |
| | Forward |
| 137 | Wheat Ms5 3D qRT-PCR primer |
| | Reverse |
| 138 | Wheat 3A marker MP0061 KASP |
| | primer Allele-specific forward primer X |
| 139 | Wheat 3A marker MP0061 KASP |
| | primer Allele-specific forward primer Y |
| 140 | Wheat 3A marker MP0061 KASP |
| | reverse primer |
| 141 | Wheat 3A marker MP0070 KASP |
| | primer Allele-specific forward primer X |
| 142 | Wheat 3A marker MP0070 KASP |
| | primer Allele-specific forward primer Y |
| 143 | Wheat 3A marker MP0070 KASP |
| | reverse primer |
| 144 | Wheat 3A marker MP0079 KASP |
| | primer Allele-specific forward primer X |
| 145 | Wheat 3A marker MP0079 KASP |
| | primer Allele-specific forward primer Y |
| 146 | Wheat 3A marker MP0079 KASP |
| | reverse primer |
| 147 | Wheat 3A marker MP0090 KASP |
| | primer Allele-specific forward primer X |
| 148 | Wheat 3A marker MP0090 KASP |
| | primer Allele-specific forward primer Y |
| 149 | Wheat 3A marker MP0090 KASP |
| | reverse primer |
| 150 | Wheat 3A marker MP0091 KASP |
| | primer Allele-specific forward primer X |
| 151 | Wheat 3A marker MP0091 KASP |
| | primer Allele-specific forward primer Y |
| 152 | Wheat 3A marker MP0091 KASP |
| | reverse primer |
| 153 | Wheat 3A marker MP0156 KASP |
| | primer Allele-specific forward primer X |
| 154 | Wheat 3A marker MP0156 KASP |
| | primer Allele-specific forward primer Y |
| 155 | Wheat 3A marker MP0156 KASP |
| | reverse primer |
| 156 | Wheat 3A marker MP0179 KASP |
| | primer Allele-specific forward primer X |
| 157 | Wheat 3A marker MP0179 KASP |
| | primer Allele-specific forward primer Y |
| 158 | Wheat 3A marker MP0179 KASP |
| | reverse primer |
| 159 | Wheat 3A marker MP0182 KASP |
| | primer Allele-specific forward primer X |
| 160 | Wheat 3A marker MP0182 KASP |
| | primer Allele-specific forward primer Y |
| 161 | Wheat 3A marker MP0182 KASP |
| | reverse primer |
| 162 | Wheat 3A marker MP0190 KASP |
| | primer Allele-specific forward primer X |
| 163 | Wheat 3A marker MP0190 KASP |
| | primer Allele-specific forward primer Y |
| 164 | Wheat 3A marker MP0190 KASP |
| | reverse primer |
| 165 | Wheat 3A marker MP0191 KASP |
| | primer Allele-specific forward primer X |
| 166 | Wheat 3A marker MP0191 KASP |
| | primer Allele-specific forward primer Y |
| 167 | Wheat 3A marker MP0191 KASP |
| | reverse primer |
| 168 | Wheat 3A marker MP0192 KASP |
| | primer Allele-specific forward primer X |
| 169 | Wheat 3A marker MP0192 KASP |
| | primer Allele-specific forward primer Y |
| 170 | Wheat 3A marker MP0192 KASP |
| | reverse primer |
| 171 | Wheat 3A marker MP0201 KASP |
| | primer Allele-specific forward primer X |
| 172 | Wheat 3A marker MP0201 KASP |
| | primer Allele-specific forward primer Y |
| 173 | Wheat 3A marker MP0201 KASP |
| | reverse primer |
| 174 | Wheat 3D marker MP0126 KASP |
| | primer Allele-specific forward primer X |
| 175 | Wheat 3D marker MP0126 KASP |
| | primer Allele-specific forward primer Y |
| 176 | Wheat 3D marker MP0126 KASP |
| | reverse primer |
| 177 | Wheat 3D marker MP0127 KASP |
| | primer Allele-specific forward primer X |
| 178 | Wheat 3D marker MP0127 KASP |
| | primer Allele-specific forward primer Y |
| 179 | Wheat 3D marker MP0127 KASP |
| | reverse primer |
| 180 | Wheat 3D marker MP0130 KASP |
| | primer Allele-specific forward primer X |
| 181 | Wheat 3D marker MP0130 KASP |
| | primer Allele-specific forward primer Y |
| 182 | Wheat 3D marker MP0130 KASP |
| | reverse primer |
| 183 | Wheat 3D marker MP0131 KASP |
| | primer Allele-specific forward primer X |
| 184 | Wheat 3D marker MP0131 KASP |
| | primer Allele-specific forward primer Y |
| 185 | Wheat 3D marker MP0131 KASP |
| | reverse primer |
| 186 | Wheat 3D marker MP0211 KASP |
| | primer Allele-specific forward primer X |
| 187 | Wheat 3D marker MP0211 KASP |
| | primer Allele-specific forward primer Y |
| 188 | Wheat 3D marker MP0211 KASP |
| | reverse primer |
| 189 | Wheat 3D marker MP0212 KASP |
| | primer Allele-specific forward primer X |
| 190 | Wheat 3D marker MP0212 KASP |
| | primer Allele-specific forward primer Y |
| 191 | Wheat 3D marker MP0212 KASP |
| | reverse primer |
| 192 | Wheat 3D marker MP0215 KASP |
| | primer Allele-specific forward primer X |
| 193 | Wheat 3D marker MP0215 KASP |
| | primer Allele-specific forward primer Y |
| 194 | Wheat 3D marker MP0215 KASP |
| | reverse primer |
| 195 | Wheat 3D marker MP0216 KASP |
| | primer Allele-specific forward primer X |
| 196 | Wheat 3D marker MP0216 KASP |
| | primer Allele-specific forward primer Y |
| 197 | Wheat 3D marker MP0216 KASP |
| | reverse primer |
| 198 | Synthesized Ms5 3A genomic (exon- |
| | intron) (Example 11) |
| 199 | Wheat Ms5 B genomic 2 (exon-intron) |
| 200 | Wheat Ms5 B promoter2 |
| 201 | Wheat Ms5 B terminator2 |
| 202 | Wheat U6 polIII promoter |
| 203 | Bar gene RbcsT terminator fusion |
| 204 | gRNA scaffold |
| 205 | Maize ubiquitin promoter |
| 206 | Rice codon-optimised Cas9 gene |
| 207 | Sorghum bicolor actin terminator |
| 208 | CaMV 35S enhancer |
| 209 | LTP2 promoter |
| 210 | DsRed2(Alt1) gene |
| 211 | PINII terminator |
| 212 | Maize Ubiquitin 1 promoter with |
| | modified first intron |

An isolated Ms1 polynucleotide comprising: (i) a nucleic acid sequence encoding a polypeptide having an amino acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the amino acid sequence of SEQ ID NO: 4, 9, or 14; or (ii) a full complement of the nucleic acid sequence of (i), wherein the full complement and the nucleic acid sequence of (i) consist of the same number of nucleotides and are 100% complementary.

An isolated Ms1 polypeptide having an amino acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the amino acid sequence of SEQ ID NO: 4, 9, or 14.

An isolated Ms1 polynucleotide comprising (i) a nucleic acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the nucleic acid sequence of SEQ ID NO: 1-3, 5-8, 10-13, and 15 and combinations thereof; or (ii) a full complement of the nucleic acid sequence of (i).

An isolated Ms1 polynucleotide comprising a nucleotide sequence, wherein the nucleotide sequence is hybridizable under stringent conditions with a DNA molecule comprising the full complement of SEQ ID NO: 1-3, 5-8, 10-13, and 15. The isolated MS1 protein of the present disclosure may also be a protein which is encoded by a nucleic acid comprising a nucleotide sequence hybridizable under stringent conditions with the complementary strand of the nucleotide sequence of SEQ ID NO:1, 3, 6, or 8.

An isolated Ms1 polynucleotide comprising a nucleotide sequence, wherein the nucleotide sequence is derived from SEQ ID NO: 1-3, 5-8, 10-13, and 15 by alteration of one or more nucleotides by at least one method selected from the group consisting of: deletion, substitution, addition and insertion.

An isolated Ms1 polynucleotide comprising a nucleotide sequence, wherein the nucleotide sequence corresponds to an allele of SEQ ID NO: 1-3, 5-8, 10-13, and 15.

As used herein, “TaLTPG2” is used interchangeably with “Ms5”. See, for example, Example 7 herein. An isolated Ms5 polynucleotide comprising: (i) a nucleic acid sequence encoding a polypeptide having an amino acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the amino acid sequence of SEQ ID NO: 19, 25-26, 33-34, 39, 44, 49, or 54; or (ii) a full complement of the nucleic acid sequence of (i), wherein the full complement and the nucleic acid sequence of (i) consist of the same number of nucleotides and are 100% complementary.

An isolated Ms5 polypeptide having an amino acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the amino acid sequence of SEQ ID NO: 19, 25-26, 33-34, 39, 44, 49, or 54.

An isolated Ms5 polynucleotide comprising (i) a nucleic acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when aligned with the nucleic acid sequence of SEQ ID NO: 16-18, 20-24, 27, 32, 35-38, 40-43, 45-48, 50-53, 55, or 199-201 and combinations thereof; or (ii) a full complement of the nucleic acid sequence of (i).

An isolated Ms5 polynucleotide comprising a nucleotide sequence, wherein the nucleotide sequence is hybridizable under stringent conditions with a DNA molecule comprising the full complement of SEQ ID NO: 16-18, 20-24, 27, 32, 35-38, 40-43, 45-48, 50-53, 55, or 199-201. The isolated MS5 protein of the present disclosure may also be a protein which is encoded by a nucleic acid comprising a nucleotide sequence hybridizable under stringent conditions with the complementary strand of the nucleotide sequence of SEQ ID NO: 16-18, 20-24, 27, 32, 35-38, 40-43, 45-48, 50-53, 55, or 199-201.

An isolated Ms5 polynucleotide comprising a nucleotide sequence, wherein the nucleotide sequence is derived from SEQ ID NO: 16-18, 20-24, 27, 32, 35-38, 40-43, 45-48, 50-53, 55, or 199-201 by alteration of one or more nucleotides by at least one method selected from the group consisting of: deletion, substitution, addition and insertion.

Any of the Ms1 or Ms5 polynucleotides and polypeptide described herein and known in the art may be utilized in any methods and compositions of the present disclosure.

Because the genetic modification is introduced at a target site located in or near a male fertility gene of Ms1 or Ms5, it is not necessary to screen a population of thousands of plants carrying random mutations, such as those resulting from chemical mutagenesis, in order to identify a plant with the introduced genetic modification. Therefore, the need to backcross a plant to remove undesired mutations that are not the introduced genetic modification is eliminated or at least reduced.

Described herein are compositions and methods for producing male-sterile plants that introduce a genetic modification into a male fertility gene locus of Ms1 or Ms5 in the plant genome in a plant cell and obtaining a plant from that plant cell. The methods may employ a guide RNA/Cas endonuclease system, wherein the Cas endonuclease is guided by the guide RNA to recognize and optionally introduce a double strand break at a specific target site into the genome of a cell. The guide RNA/Cas endonuclease system provides for an effective system for modifying target sites within the genome of a plant, plant cell or seed. The target site recognized by a Cas endonuclease may be located within or outside the Ms1 or Ms5 ponucleotide sequence, for example, within or outside the Ms1 or Ms5 gene locus.

In one embodiment, the method comprises a method for producing a male-sterile plant, the method comprising: a) obtaining a first plant comprising at least one Cas endonuclease capable of introducing a double strand break at a genomic target site located in a male fertility gene locus of Ms1 or Ms5 in the plant genome; b) obtaining a second plant comprising a guide RNA that is capable of forming a complex with the Cas endonuclease of (a),c) crossing the first plant of (a) with the second plant of (b); d) evaluating the progeny of (c) for an alteration in the target site; and e) selecting a progeny plant that is male-sterile.

Compositions and methods are also provided for editing a nucleotide sequence in the genome of a cell. In one embodiment, the disclosure describes a method for editing a nucleotide sequence located in or near a male fertility gene of Ms1 or Ms5 in the genome of a plant cell, the method comprising providing a guide RNA, a polynucleotide modification template, and at least one maize optimized Cas9 endonuclease to a plant cell, wherein the maize optimized Cas9 endonuclease is capable of introducing a double-strand break at a target site in the plant genome, wherein said polynucleotide modification template includes at least one nucleotide modification of said nucleotide sequence. The nucleotide to be edited (the nucleotide sequence of interest) can be located within or outside a target site located in or near a male fertility gene of Ms1 or Ms5 that is recognized and cleaved by a Cas endonuclease. Cells include, but are not limited to, plant cells as well as plants and seeds produced by the methods described herein.

Compositions and methods are also provided for methods of modifying the male-fertility of a plant, the method comprising introducing at least one guide RNA, at least one polynucleotide modification template and at least one Cas endonuclease into a cell. The Cas endonuclease introduces a double-strand break at a target site located in or near a Ms1 or Ms5 gene in the genome of said plant cell and the polynucleotide modification template comprises at least one nucleotide modification of a nucleotide sequence at the target site located in or near a male fertility gene of Ms1 or Ms5 that decreases the expression level of the Ms1 or Ms5 gene, to produce a male-sterile plant.

In another embodiment, the methods include selecting a male-sterile plant, the method comprising selecting at least one male-sterile plant that comprises the introduced genetic modification(s) in at least one or more of the endogenous Ms1 or Ms5 polynucleotide sequences or Ms1 or Ms5 gene locus. Also provided is a plant cell or plant or seed obtained or produced from the methods described herein.

The plant in the embodiments described herein is a monocot or a dicot. More specifically, the monocot is selected from the group consisting of maize, rice, sorghum, rye, barley, wheat, millet, oats, sugarcane, turfgrass, or switchgrass. The dicot is selected from the group consisting of soybean, canola, alfalfa, sunflower, cotton, tobacco, peanut, potato, tobacco, Arabidopsis, or safflower.

CRISPR loci (Clustered Regularly Interspaced Short Palindromic Repeats) (also known as SPIDRs—SPacer Interspersed Direct Repeats) constitute a family of recently described DNA loci. CRISPR loci consist of short and highly conserved DNA repeats (typically 24 to 40 bp, repeated from 1 to 140 times—also referred to as CRISPR-repeats) which are partially palindromic. The repeated sequences (usually specific to a species) are interspaced by variable sequences of constant length (typically 20 to 58 by depending on the CRISPR locus (WO2007/025097 published Mar. 1, 2007).

A Cas gene includes a gene that is generally coupled, associated or close to or in the vicinity of flanking CRISPR loci. The terms “Cas gene”, “CRISPR-associated (Cas) gene” are used interchangeably herein. A comprehensive review of the Cas protein family is presented in Haft et al. (2005) Computational Biology, PLOS Comput Biol 1 (6): e60. doi: 10.1371/journal.pcbi.0010060.

Cas endonuclease relates to a Cas protein encoded by a Cas gene, wherein said Cas protein is capable of introducing a double strand break into a DNA target sequence. The Cas endonuclease is guided by the guide polynucleotide to recognize and optionally introduce a double strand break at a specific target site into the genome of a cell. As used herein, the tem “guide polynucleotide/Cas endonuclease system” includes a complex of a Cas endonuclease and a guide polynucleotide that is capable of introducing a double strand break into a DNA target sequence. The Cas endonuclease unwinds the DNA duplex in close proximity of the genomic target site and cleaves both DNA strands upon recognition of a target sequence by a guide RNA, but only if the correct protospacer-adjacent motif (PAM) is approximately oriented at the 3′ end of the target sequence.

In one embodiment, the Cas endonuclease gene is a Cas9 endonuclease, such as but not limited to, Cas9 genes listed in SEQ ID NOs: 462, 474, 489, 494, 499, 505, and 518 of WO2007/025097 published Mar. 1, 2007, and incorporated herein by reference. In another embodiment, the Cas endonuclease gene is plant optimized Cas9 endonuclease, for example, codon-optimized for expression in maize, wheat, or soybean. In another embodiment, the Cas endonuclease gene is operably linked to a SV40 nuclear targeting signal upstream of the Cas codon region and a bipartite VirD2 nuclear localization signal (Tinland et al. (1992) Proc. Natl. Acad. Sci. USA 89:7442-6) downstream of the Cas codon region. As used herein, “operably linked” is intended to mean a functional linkage between two or more elements. For example, an operable linkage between a polynucleotide of interest and a regulatory sequence (e.g., a promoter) is a functional link that allows for expression of the polynucleotide of interest. Operably linked elements may be contiguous or non-contiguous. When used to refer to the joining of two protein coding regions, operably linked means that the coding regions are in the same reading frame. In one embodiment, the Cas endonuclease gene is a Cas9 endonuclease gene of SEQ ID NO:1, 124, 212, 213, 214, 215, 216, 193 or nucleotides 2037-6329 of SEQ ID NO:5, or any functional fragment or variant thereof of US publication number 20160208272, published Jul. 26, 2016, and incorporated herein by reference.

The terms “functional fragment”, “fragment that is functionally equivalent” and “functionally equivalent fragment” are used interchangeably herein. These terms refer to a portion or subsequence of the polypeptide sequence of the present disclosure in which the polypeptide's native function is retained.

The terms “functional variant”, “Variant that is functionally equivalent” and “functionally equivalent variant” are used interchangeably herein. These terms refer to a variant of a polypeptide of the present disclosure in which the polypeptide's native function is retained. Fragments and variants can be obtained via methods such as site-directed mutagenesis and synthetic construction.

The Cas endonuclease gene may be a plant codon optimized Streptococcus pyogenes Cas9 gene that can recognize any genomic sequence of the form N (12-30) NGG can in principle be targeted.

The Cas endonuclease may be introduced directly into a cell by any method known in the art, for example, but not limited to transient introduction methods, transfection and/or topical application, including those described in US publication number 20160208272, published Jul. 26, 2016, and incorporated herein by reference. The type II CRISPR/Cas system from bacteria employs a crRNA and tracrRNA to guide the Cas endonuclease to its DNA target. The crRNA (CRISPR RNA) contains the region complementary to one strand of the double strand DNA target and base pairs with the tracrRNA (trans-activating CRISPR RNA) forming a RNA duplex that directs the Cas endonuclease to cleave the DNA target.

As used herein, the term “guide RNA” relates to a synthetic fusion of two RNA molecules, a crRNA (CRISPR RNA) comprising a variable targeting domain, and a tracrRNA. In one embodiment, the guide RNA comprises a variable targeting domain of 12 to 30 nucleotide sequences and a RNA fragment that can interact with a Cas endonuclease.

As used herein, the term “guide polynucleotide”, relates to a polynucleotide sequence that can form a complex with a Cas endonuclease and enables the Cas endonuclease to recognize and optionally cleave a DNA target site. The guide polynucleotide can be a single molecule or a double molecule. The guide polynucleotide sequence can be a RNA sequence, a DNA sequence, or a combination thereof (a RNA-DNA combination sequence). A guide polynucleotide that solely comprises ribonucleic acids is also referred to as a “guide RNA”.

The guide polynucleotide can be a double molecule (also referred to as duplex guide polynucleotide) comprising a first nucleotide sequence domain (referred to as Variable Targeting domain or VT domain) that is complementary to a nucleotide sequence in a target DNA and a second nucleotide sequence domain (referred to as Cas endonuclease recognition domain or CER domain) that interacts with a Cas endonuclease polypeptide. The CER domain of the double molecule guide polynucleotide comprises two separate molecules that are hybridized along a region of complementarity. The two separate molecules can be RNA, DNA, and/or RNA-DNA-combination sequences. In some embodiments, the first molecule of the duplex guide polynucleotide comprising a VT domain linked to a CER domain is referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). The crNucleotide can comprise a fragment of the cRNA naturally occurring in Bacteria and Archaea. In one embodiment, the size of the fragment of the cRNA naturally occurring in Bacteria and Archaea that is present in a crNucleotide disclosed herein can range from, but is not limited to, 2, 3, 4, 5, 6, 7, 8, 9, 10, 11, 12, 13, 14, 15, 16, 17, 18, 19, 20 or more nucleotides. In one example, the second molecule of the duplex guide polynucleotide comprising a CER domain is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides. In one example, the RNA that guides the RNA/Cas9 endonuclease complex, is a duplexed RNA comprising a duplex crRNA-tracrRNA.

The guide polynucleotide can also be a single molecule comprising a first nucleotide sequence domain (referred to as Variable Targeting domain or VT domain) that is complementary to a nucleotide sequence in a target DNA and a second nucleotide domain (referred to as Cas endonuclease recognition domain or CER domain) that interacts with a Cas endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise a RNA sequence, a DNA sequence, or a RNA-DNA-combination sequence. In some examples, the single guide polynucleotide comprises a crNucleotide (comprising a VT domain linked to a CER domain) linked to a tracrNucleotide (comprising a CER domain), wherein the linkage is a nucleotide sequence comprising a RNA sequence, a DNA sequence, or a RNA-DNA combination sequence. The single guide polynucleotide being comprised of sequences from the crNucleotide and tracrNucleotide may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). The single guide RNA may comprise a cRNA or cRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a plant genomic target site, enabling the Cas endonuclease to introduce a double strand break into the genomic target site. One aspect of using a single guide polynucleotide versus a duplex guide polynucleotide is that only one expression cassette needs to be made to express the single guide polynucleotide.

The term “variable targeting domain” or “VT domain” is used interchangeably herein and includes a nucleotide sequence that is complementary to one strand (nucleotide sequence) of a double strand DNA target site. The % complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable target domain can be at least 12, 13, 14, 15, 16, 17, 18, 19, 20, 21, 22, 23, 24, 25, 26, 27, 28, 29 or 30 nucleotides in length. In some examples, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The term “Cas endonuclease recognition domain” or “CER domain” of a guide polynucleotide is used interchangeably herein and includes a nucleotide sequence (such as a second nucleotide sequence domain of a guide polynucleotide), that interacts with a Cas endonuclease polypeptide. The CER domain can be composed of a DNA sequence, a RNA sequence, a modified DNA sequence, a modified RNA sequence (see for example modifications described herein), or any combination thereof.

The nucleotide sequence linking the crNucleotide and the tracrNucleotide of a single guide polynucleotide can comprise a RNA sequence, a DNA sequence, or a RNA-DNA combination sequence. In one example, the nucleotide sequence linking the crNucleotide and the tracrNucleotide of a single guide polynucleotide can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In another example, the nucleotide sequence linking the crNucleotide and the tracrNucleotide of a single guide polynucleotide can comprise a tetraloop sequence, such as, but not limiting to a GAAA tetraloop sequence.

Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

The guide RNA and Cas endonuclease are capable of forming a complex that enables the Cas endonuclease to introduce in the plant genome a double strand break at a DNA target site, for example, in a male fertility gene locus of Ms1 or Ms5 or within Ms1 or Ms5 polynucleotides themselves. The variable target domain may be 12, 13, 14, 15, 16, 17, 18, 19, 20, 21, 22, 23, 24, 25, 26, 27, 28, 29 or 30 nucleotides in length.

In some approaches, the guide RNA comprises a cRNA (or cRNA fragment) and a tracrRNA (or tracrRNA fragment) of the type II CRISPR/Cas system that can form a complex with a type II Cas endonuclease, wherein said guide RNA/Cas endonuclease complex can direct the Cas endonuclease to a plant genomic target site, enabling the Cas endonuclease to introduce a double strand break into the genomic target site.

The guide RNA can be introduced into a plant or plant cell directly using any method known in the art such as, but not limited to, particle bombardment or topical applications, for example, as described in US publication number 20160208272, published Jul. 26, 2016, and incorporated herein by reference.

The guide RNA may be introduced indirectly by introducing a recombinant DNA molecule comprising the corresponding guide DNA sequence operably linked to a plant specific promoter that is capable of transcribing the guide RNA in said plant cell. The term “corresponding guide DNA” includes a DNA molecule that is identical to the RNA molecule but has a “T” substituted for each “U” of the RNA molecule. The guide RNA may be introduced via particle bombardment or Agrobacterium transformation of a recombinant DNA construct comprising the corresponding guide DNA operably linked to a plant U6 polymerase III promoter.

The RNA that guides the RNA/Cas9 endonuclease complex, may be a duplexed RNA comprising a duplex crRNA-tracrRNA. One advantage of using a guide RNA versus a duplexed crRNA-tracrRNA is that only one expression cassette needs to be made to express the fused guide RNA.

The terms “target site”, “target sequence”, “target DNA”, “target locus”, “genomic target site”, “genomic target sequence”, and “genomic target locus” are used interchangeably herein and refer to a polynucleotide sequence in the genome (including choloroplastic and mitochondrial DNA) of a plant cell at which a double-strand break is induced in the plant cell genome by a Cas endonuclease. The target site can be an endogenous site in the plant genome, or alternatively, the target site can be heterologous to the plant and thereby not be naturally occurring in the genome, or the target site can be found in a heterologous genomic location compared to where it occurs in nature. As used herein, terms “endogenous target sequence” and “native target sequence” are used interchangeable herein to refer to a target sequence that is endogenous or native to the genome of a plant and is at the endogenous or native position of that target sequence in the genome of the plant.

The target site may be similar to a DNA recognition site or target site that that is specifically recognized and/or bound by a double-strand break inducing agent such as a LIG3-4 endonuclease (US patent publication 2009-0133152 A1 (published May 21, 2009) or a meganuclease (U.S. patent publication 20150184194 published Jul. 2, 2015).

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii). For example, the methods and compositions described herein may be used to produce a Ms1 or Ms5 modified target site which confers male-sterility to the plant containing the modified Ms1 or Ms5 target site or introduced genetic modification.

Methods for modifying a plant genomic target site are disclosed herein.

In another embodiment, the method includes modifying a target site located in or near a Ms1 or Ms5 gene in the genome of a plant cell, the method comprising introducing a guide RNA into a plant cell having a Cas endonuclease, wherein said guide RNA and Cas endonuclease are capable of forming a complex that enables the Cas endonuclease to introduce a double strand break at said target site.

Also provided is a method for modifying a target site in the genome of a plant cell, the method comprising introducing a guide RNA and a Cas endonuclease into said plant, wherein said guide RNA and Cas endonuclease are capable of forming a complex that enables the Cas endonuclease to introduce a double strand break at said target site. In some embodiments, the guideRNA can simultaneously modify the same target site in multiple genomes in the plant cell or plant. See, for example, Example 3, demonstrating the generation of ms1 mutations in the b genome in wheat using Cas9 technology. Table 2 provided herein shows exemplary DNA versions of wheat guideRNAs and target sequences for making Ms1 or Ms5 mutations in wheat genomes to confer male-sterility to a plant. Additionally, many of the target sequences listed for wheat are consensus sequences so that each genome (A, B, or D) can be modified simultaneously using the same guideRNA to produce the genetic modification. For example, the target sequences of SEQ ID NOs: 57-59, 61-64, and 67-81 shown in Table 2 were selected as each site is a consensus region found in all three (A, B, and D) genomes in wheat. In some embodiments, only one genome in wheat is targeted, see, for example, SEQ ID NOs: 56, 60, and 65-66 specifically targeting the wheat B genome. As shown in Example 3 herein, targeting the B genome alone is sufficient to cause male-sterility of the wheat plant.

Further provided is a method for modifying a target site in or near a Ms1 or Ms5 gene in the genome of a plant cell, the method comprising: a) introducing into a plant cell a guide RNA comprising a variable targeting domain and a Cas endonuclease, wherein said guide RNA and Cas endonuclease are capable of forming a complex that enables the Cas endonuclease to introduce a double strand break at said target site; and, b) identifying at least one plant cell that has a modification at said target, wherein the modification includes at least one deletion or substitution of one or more nucleotides in said target site. A plant derived the modified plant cell is male-sterile.

Further provided, a method for modifying a target DNA sequence in or near a Ms1 or Ms5 gene in the genome of a plant cell, the method comprising: a) introducing into a plant cell a first recombinant DNA construct capable of expressing a guide RNA and a second recombinant DNA construct capable of expressing a Cas endonuclease, wherein said guide RNA and Cas endonuclease are capable of forming a complex that enables the Cas endonuclease to introduce a double strand break at said target site; and, b) identifying at least one plant cell that has a modification at said target, wherein the modification includes at least one deletion or substitution of one or more nucleotides in said target site and the modification confers male-sterility to a plant derived from the modified plant cell.

The length of the target site can vary, and includes, for example, target sites that are at least 12, 13, 14, 15, 16, 17, 18, 19, 20, 21, 22, 23, 24, 25, 26, 27, 28, 29, 30 or more nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other Cases, the incisions could be staggered to produce single-stranded overhangs, also called “sticky ends”, which can be either 5′ overhangs, or 3′ overhangs.

In some embodiment, the genomic target site capable of being cleaved by a Cas endonuclease comprises a 12 to 30 nucleotide fragment of a male fertility gene. Exemplary male fertility genes for use in the compositions and methods described here include but are not limited to MS1 or MS5. In some embodiments, the MS1 or MS5 fertility genes or gene loci to be targeted are from wheat, barley, maize, rice, sorghum, rye, millet, oats, sugarcane, turfgrass, switchgrass, soybean, canola, alfalfa, sunflower, cotton, tobacco, peanut, potato, tobacco, Arabidopsis, or safflower.

TABLE 2
Exemplary Guide RNAs and Target Sequence Description

| | | | | Relative | | | |
|---|---|---|---|---|---|---|---|
| Target | Target Sequence | Start | | DSB | | | |
| Sequence | (5′-3′) and | Position | Relative End | Position | PAM | DNA version of | Target |
| Designation | target strand | (bp) | Position (bp) | (bp) | Sequence | Guide RNA | Site |
| Wheat Ms1 | CGCCACCAGCAGCAGC | Position | Position 33 | Between | CGG | CGCCACCAGCAGCAGC | Exon 1 |
| CR1 | CCGCGG- | 12 of | of SEQ ID | Position 17 | | CCG | |
| | complementary | SEQ ID | NO: 6 | and 18 of | | (SEQ ID NO: 82) | |
| | (SEQ ID NO: 56) | NO: 6 | | SEQ ID NO: 6 | | | |
| Wheat Ms1 | GGGCGGGGGGCTGCTGA | Position | Position | Between | CGG | GGGCGGGGGGCTGCTG | Exon 2 |
| CR2 | CGACGG- | 1416 of | 1438 of SEQ | Position | | ACGA | |
| | Complementary | SEQ ID | ID NO: 6 | 1421 and | | (SEQ ID NO: 83) | |
| | (SEQ ID NO: 57) | NO: 6 | | 1422 of SEQ | | | |
| | | | | ID NO: 6 | | | |
| Wheat Ms1 | GTCGTCCCCGCCGCCGT | Position | Position 1668 | Between | AGG | GTCGTCCCCGCCGCCG | Exon 3 |
| CR3 | CCCAGG- | 1646 of | of SEQ ID | Position | | TCCC | |
| | Sense | SEQ ID | NO: 6 | 1662 and | | (SEQ ID NO: 84) | |
| | (SEQ ID NO: 58) | NO: 6 | | 1663 of SEQ | | | |
| | | | | ID NO: 6 | | | |
| Wheat Ms1 | GACGAAGAAGAAGGCCG | Position | Position | Between | TGG | GACGAAGAAGAAGGCC | Exon 3 |
| CR4 | CCTTGG- | 1798 of | 1820 of SEQ | Position | | GCCT | |
| | Complementary | SEQ ID | ID NO: 6 | 1803 and | | (SEQ ID NO: 85) | |
| | (SEQ ID NO: 59) | NO: 6 | | 1804 of SEQ | | | |
| | | | | ID NO: 6 | | | |
| Wheat Ms1 | GCCCACGGCGCCGTCCA | Position | Position | Between | CGG | GCCCACGGCGCCGTCC | Exon 3 |
| CR5 | AGGCGG- | 1784 of | 1806 of SEQ | Position | | AAGG | |
| | Sense | SEQ ID | ID NO: 6 | 1800 and | | (SEQ ID NO: 86) | |
| | (SEQ ID NO: 60) | NO: 6 | | 1801 of SEQ | | | |
| | | | | ID NO: 6 | | | |
| Wheat Ms1 | GGCCGTGGCGACGAAGA | Position | Position | Between | AGG | GGCCGTGGCGACGAAG | Exon 3 |
| CR6 | AGAAGG- | 1807 of | 1829 of SEQ | Position | | AAGA | |
| | Complementary | SEQ ID | ID NO: 6 | 1812 and | | (SEQ ID NO: 87) | |
| | (SEQ ID NO: 61) | NO: 6 | | 1813 of SEQ | | | |
| | | | | ID NO: 6 | | | |
| Wheat Ms1 | GTAGAGGCCGAGCATGG | Position | Position | Between | TGG | GTAGAGGCCGAGCATG | Exon 3 |
| CR7 | CCGTGG- | 1822 of | 1844 of SEQ | Position | | GCCG | |
| | Complementary | SEQ ID | ID NO: 6 | 1827 and | | (SEQ ID NO: 88) | |
| | (SEQ ID NO: 62) | NO: 6 | | 1828 of SEQ | | | |
| | | | | ID NO: 6 | | | |
| Wheat Ms1 | GGCCTTCTTCTTCGTCG | Position | Position | Between | CGG | GGCCTTCTTCTTCGTC | Exon 3 |
| CR8 | CCACGG- | 1805 of | 1827 of SEQ | Position | | GCCA | |
| | Sense | SEQ ID | ID NO: 6 | 1821 and | | (SEQ ID NO: 89) | |
| | (SEQ ID NO: 63) | NO: 6 | | 1822 of SEQ | | | |
| | | | | ID NO: 6 | | | |
| Wheat Ms1 | GATGATGTAGAGGCCGA | Position | Position | Between | TGG | GATGATGTAGAGGCCG | Exon 3 |
| CR9 | GCATGG- | 1828 of | 1850 of SEQ | Position | | AGCA | |
| | Complementary | SEQ ID | ID NO: 6 | 1833 and | | (SEQ ID NO: 90) | |
| | (SEQ ID NO: 64) | NO: 6 | | 1834 of SEQ | | | |
| | | | | ID NO: 6 | | | |
| Wheat Ms1 | GAGATCCCGCGGGCTGC | Position 6 | Position 28 | Between | TGG | (SEQ ID NO: 92) | Exon1 |
| CR10 | TGCTGG- | of SEQ ID | of SEQ ID | Position 22 | | GAGATCCCGCGGGCTG | |
| | sense | NO: 6 | NO: 6 | and 23 of | | CTGC | |
| | (SEQ ID NO: 65) | | | SEQ ID NO: 6 | | (SEQ ID NO: 91) | |
| Wheat Ms1 | GCTGCTGGCGGCGCTGC | Position | Position 58 | Between | CGG | GCTGCTGGCGGCGCTG | Exon 1 |
| CR11 | TGCCGG- | 36 of | of SEQ ID | Position 52 | | CTGC | |
| | sense | SEQ ID | NO: 6 | and 53 of | | (SEQ ID NO: 92) | |
| | (SEQ ID NO: 66) | NO: 6 | | SEQ ID NO: 6 | | | |
| | | | | Start and | | | |
| | | | | End | | | |
| Target | Target Sequence | Start | | Positions of | | | |
| Sequence | (5′-3′) | Position | Relative End | Target | PAM | DNA version of | Target |
| Designation | and target strand | (bp) | Position (bp) | Sequence | Sequence | Guide RNA | Site |
| Wheat Ms5 | GCACGGCGAGAAGGAC | 110 | 132 | Position | TGG | GCACGGCGAGAAGGAC | Exon 1 |
| CR1 | ACGATGG- | | | relative to | | ACGA | |
| | Complementary | | | ATG in SEQ | | (SEQ ID NO: 93) | |
| | (SEQ ID NO: 67) | | | ID NO: 16 | | | |
| Wheat Ms5 | GCAGCAGGCGCTGGTGG | 176 | 198 | Position | CGG | GCAGCAGGCGCTGGTG | Exon 1 |
| CR2 | GCGCGG- | | | relative to | | GGCG | |
| | Complementary | | | ATG in SEQ | | (SEQ ID NO: 94) | |
| | (SEQ ID NO: 68) | | | ID NO: 16 | | | |
| Wheat Ms5 | GCCGCGCAGCAGGCGCT | 181 | 203 | Position | GGG | GCCGCGCAGCAGGCGC | Exon 1 |
| CR3 | GGTGGG- | | | relative to | | TGGT | |
| | Complementary | | | ATG in SEQ | | (SEQ ID NO: 95) | |
| | (SEQ ID NO: 69) | | | ID NO: 16 | | | |
| Wheat Ms5 | GAACGCCGCGCAGCAGG | 185 | 207 | Position | TGG | GAACGCCGCGCAGCAG | Exon 1 |
| CR4 | CGCTGG- | | | relative to | | GCGC | |
| | Complementary | | | ATG in SEQ | | (SEQ ID NO: 96) | |
| | (SEQ ID NO: 70) | | | ID NO: 16 | | | |
| Wheat Ms5 | GCGCAGGAACGCCGCGC | 191 | 203 | Position | AGG | GCGCAGGAACGCCGCG | Exon 1 |
| CR5 | AGCAGG- | | | relative to | | CAGC | |
| | Complementary | | | ATG in SEQ | | (SEQ ID NO: 97) | |
| | (SEQ ID NO: 71) | | | ID NO: 16 | | | |
| Wheat Ms5 | GCCCACCAGCGCCTGCT | 180 | 202 | Position | CGG | GCCCACCAGCGCCTGC | Exon 1 |
| CR6 | GCGCGG- | | | relative to | | TGCG | |
| | Sense | | | ATG in SEQ | | (SEQ ID NO: 98) | |
| | (SEQ ID NO: 72) | | | ID NO: 16 | | | |
| Wheat Ms5 | GCCGCCTTCGCCGTCCC | 221 | 243 | Position | AGG | GCCGCCTTCGCCGTCC | Exon 1 |
| CR7 | CGGAGG- | | | relative to | | CCGG | |
| | Complementary | | | ATG in SEQ | | (SEQ ID NO: 99) | |
| | (SEQ ID NO: 73) | | | ID NO: 16 | | | |
| Wheat Ms5 | GGGGACGGCGAAGGCGG | 226 | 248 | Position | AGG | GGGGACGGCGAAGGCG | Exon 1 |
| CR8 | CGGAGG- | | | relative to | | GCGG | |
| | Sense | | | ATG in SEQ | | (SEQ ID NO: 100) | |
| | (SEQ ID NO: 74) | | | ID NO: 16 | | | |
| Wheat Ms5 | GGGACGGCGAAGGCGGC | 227 | 249 | Position | GGG | GGGACGGCGAAGGCGG | Exon 1 |
| CR9 | GGAGGG- | | | relative to | | CGGA | |
| | Sense | | | ATG in SEQ | | (SEQ ID NO: 101) | |
| | (SEQ ID NO: 75) | | | ID NO: 16 | | | |
| Wheat Ms5 | GGACGGCGAAGGCGGCG | 228 | 250 | Position | GGG | GGACGGCGAAGGCGGC | Exon 1 |
| CR10 | GAGGGG- | | | relative to | | GGAG | |
| | Sense | | | ATG in SEQ | | (SEQ ID NO: 102) | |
| | (SEQ ID NO: 76) | | | ID NO: 16 | | | |
| Wheat Ms5 | GGCGAAGGCGGCGGAGG | 232 | 254 | Position | GGG | GGCGAAGGCGGCGGAG | Exon 1 |
| CR11 | GGAGGG- | | | relative to | | GGGA | |
| | Sense | | | ATG in SEQ | | (SEQ ID NO: 103) | |
| | (SEQ ID NO: 77) | | | ID NO: 16 | | | |
| Wheat Ms5 | GCCGAGGCGCGCGGCGT | 299 | 321 | Position | CGG | GCCGAGGCGCGCGGCG | Exon 1 |
| CR12 | CGACGG- | | | relative to | | TCGA | |
| | Complementary | | | ATG in SEQ | | (SEQ ID NO: 104) | |
| | (SEQ ID NO: 78) | | | ID NO: 16 | | | |
| Wheat Ms5 | GTTTTCGCGGAGGCGCA | 331 | 353 | Position | GGG | GTTTTCGCGGAGGCGC | Exon 1 |
| CR13 | GGTGGG- | | | relative to | | AGGT | |
| | Complementary | | | ATG in SEQ | | (SEQ ID NO: 105) | |
| | (SEQ ID NO: 79) | | | ID NO: 16 | | | |
| Wheat Ms5 | GGTTTTCGCGGAGGCGC | 332 | 354 | Position | TGG | GGTTTTCGCGGAGGCG | Exon 1 |
| CR14 | AGGTGG- | | | relative to | | CAGG | |
| | Complementary | | | ATG in SEQ | | (SEQ ID NO: 106) | |
| | (SEQ ID NO: 80) | | | ID NO: 16 | | | |
| Wheat Ms5 | GGAGGTTTTCGCGGAGG | 335 | 357 | Position | AGG | GGAGGTTTTCGCGGAG | Exon |
| CR15 | CGCAGG- | | | relative to | | GCGC | 1 |
| | Complementary | | | ATG in SEQ | | (SEQ ID NO: 107) | |
| | (SEQ ID NO: 81) | | | ID NO: 16 | | | |

TABLE 3
Description of resulting MS1 genome edits

| Target Sequence (5′-3′) | DNA version of | Target | |
|---|---|---|---|
| and target strand | Guide RNA | Site | Description of Edit |
| CGCCACCAGCAGCAGCCCGCGG- | CGCCACCAGCAGCAGCCCG | Exon | Exon 1 |
| complementary | (SEQ ID NO: 82) | 1 | Insertion of one nucleotide (A) at position |
| (SEQ ID NO: 56) | | | 18 of MS1 CDS (SEQ ID NO: 8 (by single |
| | | | guide RNA (SEQ ID NO: 82) |
| | | | CGCCACCAGCAGCAGCACCG |
| | | | (SEQ ID NO: 108) |
| CGCCACCAGCAGCAGCCCGCGG- | CGCCACCAGCAGCAGCCCG | Exon | Exon 1 |
| complementary | (SEQ ID NO: 82) | 1 | Insertion of one nucleotide (T) at position |
| (SEQ ID NO: 56) | | | 18 of MS1 CDS (SEQ ID NO: 8) (by single |
| | | | guide RNA; (SEQ ID NO: 82) |
| | | | CGCCACCAGCAGCAGCTCCG (SEQ ID |
| | | | NO: 109) |
| CGCCACCAGCAGCAGCCCGCGG- | CGCCACCAGCAGCAGCCCG | Exon | Exon 1 |
| complementary | (SEQ ID NO: 82) | 1 | Insertion of one nucleotide (C) at position |
| (SEQ ID NO: 56) | | | 18 of MS1 CDS (SEQ ID NO: 8) (by single |
| | | | guide RNA; (SEQ ID NO: 82) |
| | | | CGCCACCAGCAGCAGCCCCG (SEQ ID |
| | | | NO: 110) |
| CGCCACCAGCAGCAGCCCGCGG- | CGCCACCAGCAGCAGCCCG | Exon | Exon 1 |
| complementary | (SEQ ID NO: 82) | 1 | Deletion of 3 nucleotide at position 18 of |
| (SEQ ID NO: 56) | | | MS1 CDS (SEQ ID NO:8) (by single guide |
| | | | RNA; (SEQ ID NO: 82) |
| | | | CGCCACCAGCAGCCCG (SEQ ID NO: 111) |

Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by a Cas endonuclease. Assays to measure the double-strand break of a target site by an endonuclease are known in the art and generally measure the overall activity and specificity of the agent on DNA substrates containing recognition sites.

As used herein, a “genomic region” is a segment of a chromosome in the genome of a plant cell. The genomic region may be present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases such that the genomic region has sufficient homology to undergo homologous recombination with the corresponding region of homology.

The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the plant genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination.

The region of homology on the donor DNA can have homology to any sequence flanking the target site. While in some embodiments the regions of homology share significant sequence homology to the genomic sequence immediately flanking the target site, it is recognized that the regions of homology can be designed to have sufficient homology to regions that may be further 5′ or 3′ to the target site. In still other embodiments, the regions of homology can also have homology with a fragment of the target site along with downstream genomic regions. In one embodiment, the first region of homology further comprises a first fragment of the target site and the second region of homology comprises a second fragment of the target site, wherein the first and second fragments are dissimilar.

As used herein, “homologous recombination” includes the exchange of DNA fragments between two DNA molecules at the sites of homology. Homology-directed repair (HDR) is a mechanism in cells to repair double-stranded and single stranded DNA breaks. Homology-directed repair includes homologous recombination (HR) and single-strand annealing (SSA) (Lieber. 2010 Annu. Rev. Biochem 79:181-211). The most common form of HDR is called homologous recombination (HR), which has the longest sequence homology requirements between the donor and acceptor DNA. Other forms of HDR include single-stranded annealing (SSA) and breakage-induced replication, and these require shorter sequence homology relative to HR. Homology-directed repair at nicks (single-stranded breaks) can occur via a mechanism distinct from HDR at double-strand breaks (Davis and Maizels. PNAS (0027-8424), 111 (10), p. E924-α932.

Alteration of the genome of a plant cell, for example, through homologous recombination (HR), is a powerful tool for genetic engineering. Despite the low frequency of homologous recombination in higher plants, there are a few examples of successful homologous recombination of plant endogenous genes. The parameters for homologous recombination in plants have primarily been investigated by rescuing introduced truncated selectable marker genes. In these experiments, the homologous DNA fragments were typically between 0.3 kb to 2 kb. Observed frequencies for homologous recombination were on the order of 10⁻⁴ to 10⁻⁵. See, for example, Halfter et al., (1992) Mol Gen Genet 231:186-93; Offringa et al., (1990) EMBO J 9:3077-84; Offringa et al., (1993) Proc. Natl. Acad. Sci. USA 90:7346-50; Paszkowski et al., (1988) EMBO J 7:4021-6; Hourda and Paszkowski, (1994) Mol Gen Genet 243:106-11; and Risseeuw et al., (1995) Plant J 7:109-19.

Once a double-strand break is induced in the DNA, the cell's DNA repair mechanism is activated to repair the break. Error-prone DNA repair mechanisms can produce mutations at double-strand break sites. The most common repair mechanism to bring the broken ends together is the nonhomologous end-joining (NHEJ) pathway (Bleuyard et al., (2006) DNA Repair 5:1-12). The structural integrity of chromosomes is typically preserved by the repair, but deletions, insertions, or other rearrangements are possible (Siebert and Puchta, (2002) Plant Cell 14:1121-31; Pacher et al., (2007) Genetics 175:21-9).

Alternatively, the double-strand break can be repaired by homologous recombination between homologous DNA sequences.

### Genome Editing Using the Guide RNA/Cas Endonuclease System

Further provided is a method for modifying a target site at or near a Ms1 or Ms5 gene in the genome of a plant cell, the method comprising introducing a guide RNA and a donor DNA into a plant cell having a Cas endonuclease, wherein said guide RNA and Cas endonuclease are capable of forming a complex that enables the Cas endonuclease to introduce a double strand break at said target site, wherein said donor DNA comprises a polynucleotide of interest that when inserted confers male-sterility to a plant obtained from the modified plant cell.

As described herein, the guide RNA/Cas endonuclease system can be used in combination with a co-delivered polynucleotide modification template to allow for editing of a genomic nucleotide sequence of interest, Ms1 or Ms5, to confer male-sterility to a plant. Also, as described herein, for each embodiment that uses a guide RNA/Cas endonuclease system, a similar guide polynucleotide/Cas endonuclease system can be deployed where the guide polynucleotide does not solely comprise ribonucleic acids but wherein the guide polynucleotide comprises a combination of RNA-DNA molecules or solely comprise DNA molecules.

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, or (iv) any combination of (i)-(iii).

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

In one embodiment provided herein, the method comprises contacting a plant cell with the donor DNA and the endonuclease. Once a double-strand break is introduced in the target site by the endonuclease, the first and second regions of homology of the donor DNA can undergo homologous recombination with their corresponding genomic regions of homology resulting in exchange of DNA between the donor and the genome. As such, the provided methods result in the integration of the polynucleotide of interest of the donor DNA into the double-strand break in the target site in the plant genome so that the endogenous male fertility gene of Ms1 or Ms5 is disrupted, thereby altering the original target site and producing an altered genomic target site that confers male sterility to the plant.

The donor DNA may be introduced by any means known in the art. For example, a plant having a target site is provided. The donor DNA may be provided by any transformation method known in the art including, for example, Agrobacterium-mediated transformation or biolistic particle bombardment. The donor DNA may be present transiently in the cell or it could be introduced via a viral replicon. In the presence of the Cas endonuclease and the target site, the donor DNA is inserted into the transformed plant's genome to disrupt an endogenous male fertility gene of Ms1 or Ms5.

In one embodiment, the disclosure describes a method for editing a nucleotide sequence in the genome of a cell, the method comprising providing a guide RNA, a polynucleotide modification template, and at least one Cas endonuclease to a cell, wherein the Cas endonuclease is capable of introducing a double-strand break at a target sequence in the genome of said cell to confer male-sterility, wherein said polynucleotide modification template includes at least one nucleotide modification of said nucleotide sequence. The nucleotide to be edited can be located within or outside a target site of one or more Ms1 or Ms5 genes recognized and cleaved by a Cas endonuclease. In one embodiment, the at least one nucleotide modification is not a modification at a target site recognized and cleaved by a Cas endonuclease. In another embodiment, there are at least 1, 2, 3, 4, 5, 6, 7, 8, 9, 10, 11, 12, 13, 14, 15, 16, 17, 18, 19, 20, 21, 22, 23, 24, 25, 26, 27, 30, 40, 50, 100, 200, 300, 400, 500, 600, 700, 900 or 1000 nucleotides between the at least one nucleotide to be edited and the genomic target site.

In another embodiment of genome editing, editing of an endogenous MS1 or MS5 gene in a plant cell or plant is disclosed herein. In some embodiments, the polynucleotide modification template (male fertility gene polynucleotide modification template) includes a partial fragment of the Ms1 or Ms5 gene (and therefore does not encode a fully functional Ms1 or Ms5 polypeptide by itself).

In one embodiment of the disclosure, a wheat Ms1 or Ms5 mutant plant is produced by the method described herein, said method comprising: a) providing a guide RNA, a polynucleotide modification template and at least one Cas endonuclease to a plant cell, wherein the Cas endonuclease introduces a double strand break at a target site within a wheat Ms1 or Ms5 (male sterility 45) genomic sequence in the plant genome, wherein said polynucleotide modification template comprises at least one nucleotide modification of the Ms1 or Ms5 genomic sequence; b) obtaining a plant from the plant cell of (a); c) evaluating the plant of (b) for the presence of said at least one nucleotide modification and d) selecting a progeny plant exhibiting male sterility from the modification of the endogenous Ms1 or Ms5 gene. The nucleotide sequence to be edited may be a sequence that is endogenous to the cell that is being edited.

### Regulatory Sequence Modifications Using the Guide Polynucleotide/Cas Endonuclease System

In one example, the nucleotide sequence to be modified can be a regulatory sequence such as a promoter, for example, for an endogenous MS1 or MS5 gene in a plant cell or plant. In some examples, the promoter may be modified to include or remove an element in the promoter. In one embodiment, the guide polynucleotide/Cas endonuclease system can be used to allow for the deletion of a promoter or promoter element, wherein the promoter deletion (or promoter element deletion) results in any one of the following or any one combination of the following: a permanently inactivated gene locus, a decreased promoter activity, a decreased promoter tissue specificity, a modification of the timing or developmental progress of gene expression, a mutation of DNA binding elements and/or an addition of DNA binding elements. Promoter elements to be deleted can be, but are not limited to, promoter core elements, such as, but not limited to, a CAAT box, a CCAAT box, a Pribnow box, TATA box, and/or translational regulation sequences, promoter enhancer elements. The promoter or promoter fragment to be deleted may be endogenous to the cell that is being edited, for example, the promoter of an endogenous Ms1 or Ms5 fertility gene.

### Additional Regulatory Sequence Modifications Using the Guide Polynucleotide/Cas Endonuclease System

The guide polynucleotide/Cas endonuclease system may be used to modify or replace a regulatory sequence in the genome of a cell. A regulatory sequence is a segment of a nucleic acid molecule which is capable of increasing or decreasing the expression of specific genes within an organism and/or is capable of altering tissue specific expression of genes within an organism. Examples of regulatory sequences include, but are not limited to, 3′ UTR (untranslated region) region, 5′ UTR region, transcription activators, transcriptional enhancers transcriptions repressors, translational repressors, splicing factors, miRNAs, siRNA, artificial miRNAs, promoter elements, polyadenylation signals, and polyubiquitination sites. In one example, the editing (modification) or replacement of a regulatory element results in altered protein translation, RNA cleavage, RNA splicing, transcriptional termination or post translational modification that confers male-sterility to a plant. In one embodiment, regulatory elements can be identified within a promoter and these regulatory elements can be edited or modified do to optimize these regulatory elements for down regulation of the promoter to create a male sterile plant.

In one embodiment, the genomic sequence of interest to be modified is an intron or UTR site, wherein the modification consist of inserting at least one microRNA into said intron or UTR site, wherein expression of the gene comprising the intron or UTR site also results in expression of said microRNA, which in turn can silence any gene targeted by the microRNA without disrupting the gene expression of the native/transgene comprising said intron.

Modifications of Splicing Sites and/or Introducing Alternate Splicing Sites Using the Guide Polynucleotide/Cas Endonuclease System

The guide polynucleotide/Cas endonuclease system can be used in combination with a co-delivered polynucleotide modification template to edit an endogenous Ms1 or Ms5 gene to introduce a canonical splice site at a described junction or any variant of a splicing site that disrupts the splicing pattern of pre-mRNA molecules so that the plant with the introduced genetic modification is male-sterile.

### Modifications of Nucleotide Sequences Encoding a Protein of Interest Using the Guide Polynucleotide/Cas Endonuclease System

In one embodiment, the guide polynucleotide/Cas endonuclease system can be used to modify or replace a coding sequence in the fertility gene locus of Ms1 or Ms5 in the genome of a plant cell, wherein the modification or replacement results in conferring male-sterility to the plant. In one embodiment, the protein knockout is due to the introduction of a stop codon into the coding sequence of interest. In one embodiment, the protein knockout is due to the deletion of a start codon into the coding sequence of interest.

### Gene Silencing by Expressing an Inverted Repeat or Antisense Using the Guide Polynucleotide/Cas Endonuclease System

In one embodiment, the guide polynucleotide/Cas endonuclease system can be used in combination with a co-delivered polynucleotide sequence to insert an inverted gene fragment into a gene of interest in the genome of an organism, wherein the insertion of the inverted gene fragment can allow for an in-vivo creation of an inverted repeat (hairpin) and results in the silencing of said endogenous gene of Ms1 or Ms5, for example, a hairpin promoter inverted repeat (pIR) directed to Ms1 or Ms5.

In one embodiment, the insertion of the inverted gene fragment can result in the formation of an in-vivo created inverted repeat (hairpin) in a native (or modified) promoter of a gene and/or in a native 5′ end of the native gene. The inverted gene fragment can further comprise an intron which can result in an enhanced silencing of the targeted endogenous Ms1 or Ms5 gene.

In one embodiment, the region of interest can be flanked by two independent guide polynucleotide/CAS endonuclease target sequences. Cutting would be done concurrently. The deletion event would be the repair of the two chromosomal ends without the region of interest. Alternative results would include inversions of the region of interest, mutations at the cut sites and duplication of the region of interest.

Furthermore, the introduced genetic modification may also comprise antisense sequences complementary to at least a portion of the messenger RNA (mRNA) for Ms1 or Ms5. Modifications of the antisense sequences may be made as long as the sequences hybridize to and interfere with expression of the corresponding mRNA. In this manner, antisense constructions having 70%, 80%, or 85% sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides, or greater may be used.

In addition, the introduced genetic modification may also be a polynucleotide arranged in the sense orientation to suppress the expression of endogenous Ms1 or Ms5 genes in plants. Methods for suppressing gene expression in plants using polynucleotides in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, generally greater than about 65% sequence identity, about 85% sequence identity, or greater than about 95% sequence identity. See, U.S. Pat. Nos. 5,283,184 and 5,034,323; herein incorporated by reference.

Protocols for introducing polynucleotides and polypeptides into plants may vary depending on the type of plant or plant cell targeted for transformation, such as monocot or dicot. Suitable methods of introducing polynucleotides and polypeptides into plant cells include but are not limited to Agrobacterium-mediated transformation (U.S. Pat. Nos. 5,563,055 and 5,981,840), direct gene transfer (Paszkowski et al., (1984) EMBO J 3:2717-22), and ballistic particle acceleration (U.S. Pat. Nos. 4,945,050; 5,879,918; 5,886,244; 5,932,782; Tomes et al., (1995) “Direct DNA Transfer into Intact Plant Cells via Microprojectile Bombardment” in Plant Cell, Tissue, and Organ Culture: Fundamental Methods, ed. Gamborg & Phillips (Springer-Verlag, Berlin). Wheat transformation may be carried out by any suitable technique known to one skilled in the art, including those described in published patent application No. 20140173781 published on Jun. 19, 2014.

### Methods for Identifying at Least One Plant Cell Comprising in its Genome the Introduced Genetic Modification at the Target Site.

Further provided are methods for identifying at least one plant cell, comprising in its genome, the introduced genetic modification at the target site. A variety of methods are available for identifying those plant cells with the introduced genetic modification into the genome at or near to the target site without using a screenable marker phenotype. Such methods can be viewed as directly analyzing a target sequence to detect any change in the target sequence, including but not limited to PCR methods, sequencing methods, nuclease digestion, Southern blots, and any combination thereof. See, for example, U.S. patent application Ser. No. 12/147,834, herein incorporated by reference. The method also comprises recovering a male-sterile plant from the plant cell having the introduced genetic modification in its genome.

The present disclosure further provides expression constructs for expressing in a plant, plant cell, or plant part a guide RNA/Cas system that is capable of binding to and creating a double strand break in a target site of the fertility gene locus of Ms1 or Ms5. In one embodiment, the expression constructs of the disclosure comprise a promoter operably linked to a nucleotide sequence encoding a Cas gene and a promoter operably linked to a guide RNA of the present disclosure. The promoter is capable of driving expression of an operably linked nucleotide sequence in a plant cell.

A promoter is a region of DNA involved in recognition and binding of RNA polymerase and other proteins to initiate transcription. A plant promoter is a promoter capable of initiating transcription in a plant cell, for a review of plant promoters, see, Potenza et al., (2004) In Vitro Cell Dev Biol 40:1-22. Constitutive promoters include, for example, the core promoter of the Rsyn7 promoter and other constitutive promoters disclosed in WO99/43838 and U.S. Pat. No. 6,072,050; the core CaMV 35S promoter (Odell et al., (1985) Nature 313:810-2); rice actin (McElroy et al., (1990) Plant Cell 2:163-71); ubiquitin (Christensen et al., (1989) Plant Mol Biol 12:619-32; Christensen et al., (1992) Plant Mol Biol 18:675-89); pEMU (Last et al., (1991) Theor Appl Genet 81:581-8); MAS (Velten et al., (1984) EMBO J 3:2723-30); ALS promoter (U.S. Pat. No. 5,659,026), and the like. Other constitutive promoters are described in, for example, U.S. Pat. Nos. 5,608,149; 5,608,144; 5,604,121; 5,569,597; 5,466,785; 5,399,680; 5,268,463; 5,608,142 and 6,177,611. In some examples, an inducible promoter may be used. Pathogen-inducible promoters induced following infection by a pathogen include, but are not limited to those regulating expression of PR proteins, SAR proteins, beta-1,3-glucanase, chitinase, etc.

### Marker Assisted Selection and Breeding of Plants

Use of markers, and/or genetically-linked nucleic acids is an effective method for selecting plant having the desired traits in breeding programs. For example, one advantage of marker-assisted selection over field evaluations is that MAS can be done at any time of year regardless of the growing season. Moreover, environmental effects are irrelevant to marker-assisted selection.

A plant breeder can advantageously use molecular markers to identify individuals containing any of the targeted genome edits by identifying marker alleles that show a statistically significant probability of co-segregation with male sterility, manifested as linkage disequilibrium. This is referred to as marker assisted selection (MAS). Thus, methods for the selection of mutant wheat plants that are homozygous or heterozygous for a mutation in the Ms1 or Ms5 gene, are also provided.

The Ms5_{FS20} mutation, is a recessive mutation of the Ms5 gene that was induced in the Chris wheat variety using ethyl methanesulfonate (Franckowiak et al. 1976. Crop Sci. and was identified in the line FS-20, also known as FS20 (Klindworth et al. 2002. Crop Sci. 42:1447-1450). The ms5_{FS20} gene was reported genetically linked to chromosome 3AL and on the basis of mapping data from crosses to Chinese Spring ditelosomic 3AL was presumed to be located at a position genetically independent of the centromere (Klindworth et al., 2002 Crop Sci. 42:1447-1450). The causal variation of the Ms5 mutation is provided herein, as are markers tightly linked to the Ms5 gene on chromosome 3AL and to TaLTPG2-3D on 3DL. Markers include but are not limited to MP0061, MP0070, MP0079, MP0090, MP091, MP0156, MP0179, MP0182, MP0190, MP0191, MP0192, MP0201, MP0126, MP0127, MP0130, MP0131, MP0211, MP0212, MP0215 and MP216; see SEQ ID NOS: 112-131. These Kompetitive Allele Specific PCR (KASP) marker amplicons, which comprise both alleles, result from a sub-genome-specific PCR using two allele-specific forward primers in combination with a single reverse primer; see SEQ ID NOS: 138-197. Allele-specific fluorescent tagging of amplicons facilitates allele detection. Such markers may be used to track ms5_{FS20} and a particular TaLTPG2-3D allele in subsequent selfing and crossing of wheat lines containing the ms5_{FS20} mutation, ensuring that the male sterility trait is advantageously inherited in a wheat breeding program.

A plant breeder can advantageously use molecular markers to identify individuals containing an Ms5 mutation by identifying marker alleles that show a statistically significant probability of co-segregation with male sterility, manifested as linkage disequilibrium. This is referred to as marker assisted selection (MAS). Thus, methods for the selection of mutant wheat plants that are homozygous or heterozygous for a mutation in the Ms5 gene, such as but not limited to ms5_{FS20} are also provided.

To perform MAS, a nucleic acid corresponding to the marker nucleic acid allele is detected in a biological sample from a plant to be selected. This detection can take the form of hybridization of a probe nucleic acid to a marker allele or amplicon thereof, e.g., using allele-specific hybridization, Southern analysis, northern analysis, in situ hybridization, hybridization of primers followed by PCR amplification of a region of the marker, DNA sequencing of a PCR amplification product, or the like. For any of the marker sequences described herein, one of ordinary skill in the art would understand how to obtain the allele at a marker locus in a particular wheat line or variety using known DNA amplification and sequencing techniques. For the purposes described herein, the lines or varieties that were used were publicly available. Hence, DNA could be obtained, and one of ordinary skill in the art could either use the provided primers or develop primers from the provided reference sequence to amplify and obtain the sequence at each marker locus from each line or variety.

After the presence (or absence) of a particular marker allele in the biological sample is verified, the plant is selected and is crossed to a second plant, optionally a wheat plant from an elite line. The progeny plants produced by the cross can be evaluated for that specific marker allele, and only those progeny plants that have the desired marker allele will be chosen.

Through marker assisted selection, a plant breeder can follow the presence of the male sterility trait through controlled crosses to obtain, when desired, a new plant containing a Ms1 or Ms5 gene mutation in either the homozygous or heterozygous state, thus maintaining the Ms1 or Ms5 gene mutations. In addition, marker assisted selection can be used to produce mutant male sterile seed parents that would be used as female, i.e. plants that need pollination by a pollen donor plant, to produce seeds of commercial interest. Alternatively, marker assisted selection could be used to produce F1 hybrids containing a Ms1 or Ms5 gene mutation in the heterozygous state.

Any of the markers provided herein, as well as any marker linked to and associated with any of those markers, can be used for marker assisted selection of the male sterility trait.

Compositions and methods for restoring male fertility to a male-sterile plant are provided. In some examples, the male-sterile plants are homozygous recessive for the fertility gene of Ms1 or Ms5. In some embodiments, the male-sterile phenotype is caused by the introduction of genetic modification of a target site located in a male fertility gene locus of Ms1 or Ms5 in a plant cell's genome. In some examples, the wheat genomes (A, B, and D) contain homologous genes that have similar gene structure and function, requiring triple mutants to result in a male-sterile phenotype. Male-sterile plants may be created using the methods and compositions described herein and those known to one skilled in the art. In some embodiments, provided herein are compositions and methods to complement and restore male fertility to wheat plants containing mutations or introduced genetic modifications in Ms1 or Ms5 genes or Ms1 or Ms5 locus.

Male-sterile plants may be restored to male fertility when a functional copy of the Ms1 or Ms5 fertility gene, from the same or different species, is used to complement the Ms1 or Ms5 mutation or introduced genetic modification. See, for example, Example 11 herein.

When the male-fertility Ms1 or Ms5 polynucleotide, fragment or variant is expressed, the plant is able to successfully produce mature pollen grains because the male-fertility polynucleotide restores the plant to a fertile condition. In some examples, the Ms1 or Ms5 polynucleotide, fragment, or variant thereof is maintained in a hemizygous state in a plant, so that only certain daughter cells will inherit the Ms1 or Ms5 polynucleotide, fragment, or variant in the process of pollen grain formation. Hemizygosity is a genetic condition existing when there is only one copy of a gene (or set of genes) with no allelic counterpart.

In some embodiments, the male-fertility Ms1 or Ms5 polynucleotide, fragment, or variants thereof, is operably linked to a promoter, to express the Ms1 or Ms5 polynucleotide, fragment, or variant and modulate, e.g, restore, the male fertility of a plant. In some examples, the Ms1 or Ms5 polynucleotide, fragment, or variant are expressed from an expression cassette. In some embodiments, the male-fertility Ms1 or Ms5 polynucleotides or expression cassette disclosed herein are maintained in a hemizygous state in a plant.

In particular embodiments, the male-fertility Ms1 or Ms5 polynucleotide, or fragment or variant thereof, is operably linked to a promoter. In certain embodiments, plant promoters can preferentially initiate transcription in certain tissues, such as stamen, anther, filament, and pollen, or developmental growth stages, such as sporogenous tissue, microspores, and microgametophyte. Such plant promoters are referred to as “tissue-preferred,” “cell-type-preferred,” or “growth-stage preferred.” Promoters which initiate transcription only in certain tissue are referred to as “tissue-specific.” Likewise, promoters which initiate transcription only at certain growth stages are referred to as “growth-stage-specific.” A “cell-type-specific” promoter drives expression only in certain cell types in one or more organs, for example, stamen cells, or individual cell types within the stamen such as anther, filament, or pollen cells.

A “male-fertility promoter” may initiate transcription exclusively or preferentially in a cell or tissue involved in the process of microsporogenesis or microgametogenesis. Male-fertility polynucleotides disclosed herein, and active fragments and variants thereof, can be operably linked to male-tissue-specific or male-tissue-preferred promoters including, for example, stamen-specific or stamen-preferred promoters, anther-specific or anther-preferred promoters, pollen-specific or pollen-preferred promoters, tapetum-specific promoters or tapetum-preferred promoters, and the like. Promoters can be selected based on the desired outcome. For example, the Ms1 or Ms5 polynucleotides can be operably linked to constitutive, tissue-preferred, growth stage-preferred, or other promoters for expression in plants.

In one embodiment, the promoters may be those which express an operably-linked Ms1 or Ms5 polynucleotide exclusively or preferentially in the male tissues of the plant. Any suitable male-fertility tissue-preferred or tissue-specific promoter may be used in the process; and any of the many such promoters known to one skilled in the art may be employed. One such promoter is the 5126 promoter, which preferentially directs expression of the polynucleotide to which it is linked to male tissue of the plants, as described in U.S. Pat. Nos. 5,837,851 and 5,689,051. Other exemplary promoters include the native promoter of Ms1 or Ms5, including those known and disclosed herein in SEQ ID NO: 2, 7, 12, 17, 22, 30, 37, 42, 47, 52 or 200.

In some examples, a termination region is operably linked to the male-fertility Ms1 or Ms5 polynucleotide, fragment or variant. In some examples, the terminator region is the native terminator of Ms1 or Ms5, including those known and disclosed herein.

Where appropriate, the Ms1 or Ms5 polynucleotides may be optimized for increased expression in the plant. That is, the Ms1 or Ms5 polynucleotides can be synthesized or altered to use plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

A male-fertility Ms1 or Ms5 polynucleotide disclosed herein can be provided in an expression cassette for expression in a plant of interest. The cassette can include 5′ and 3′ regulatory sequences operably linked to a male-fertility polynucleotide as disclosed herein. In some examples, the expression cassette includes in addition to the polynucleotide encoding the Ms1 or Ms5 polypeptide a male-gamete-disruptive polynucleotide, that is, a polynucleotide which interferes with the function, formation, or dispersal of male gametes. A male-gamete-disruptive polynucleotide can operate to prevent function, formation, or dispersal of male gametes by any of a variety of methods. By way of example but not limitation, this can include use of polynucleotides which encode a gene product such as DAM-methylase or barnase (See, for example, U.S. Pat. No. 5,792,853 or 5,689,049; PCT/EP89/00495); encode a gene product which interferes with the accumulation of starch, degrades starch, or affects osmotic balance in pollen, such as alpha-amylase (See, for example, U.S. Pat. Nos. 7,875,764; 8,013,218; 7,696,405, 8,614,367); inhibit formation of a gene product important to male gamete function, formation, or dispersal (See, for example, U.S. Pat. Nos. 5,859,341; 6,297,426). In some examples, the male-gamete-disruptive polynucleotide is operably linked to a male-tissue-preferred promoter.

When the expression cassette is introduced into the plant in a hemizygous condition, only certain daughter cells will inherit the expression cassette in the process of pollen grain formation. The daughter cells that inherit the expression cassette containing the male-fertility Ms1 or Ms5 polynucleotide will not develop into mature pollen grains due to the male-tissue-preferred expression of the stacked encoded male-gamete-disruptive gene product. Those pollen grains that do not inherit the expression cassette will continue to develop into mature pollen grains and be functional, but will not contain the male-fertility polynucleotide of the expression cassette and therefore will not transmit the male-fertility polynucleotide to progeny through pollen. See, for example, U.S. Pat. Nos. 7,875,764; 8,013,218; 7,696,405, 8,614,367, herein incorporated by reference in its entirety.

In one embodiment, the homozygous recessive condition of a male-sterile plant produced using methods described herein is maintained. A method of maintaining the homozygous recessive condition of a male-sterile plant may include fertilizing the homozygous recessive male-sterile plant with pollen from a plant expressing (1) a Ms1 or Ms5 fertility gene that when the gene is expressed in the plant restores male fertility to the male-sterile plant and (2) a polynucleotide sequence that inhibits the function or formation of viable male gametes, which are driven by promoters that preferentially expresses the sequence in male plant cells, such as male gametes. See, for example, U.S. Pat. No. 8,614,367. The progeny produced will continue to be male sterile as a result of maintaining homozygosity for the fertility gene, e.g. Ms1 or Ms5. The progeny will not contain the introduced restoring fertility gene-male gamete inhibition construct. The plant having the restorer nucleotide sequence may be self-fertilized, that is pollen from the plant transferred to the flower of the same plant to achieve the propagation of the restorer plants. Note that in referring to “self fertilization”, it includes the situation where the plant producing the pollen is fertilized with that same pollen, and the situation where two or more identical inbred plants are planted together and pollen from the identical inbred plant pollinate a different identical inbred plant. The pollen will not have the restoring transgene construct but it will be contained in 50% of the ovules (the female gamete). The seed resulting from the self-fertilization can be planted, and selection made for the seed having the restoring fertility gene-male gamete inhibition construct. Selection will allow for the identification of those plants produced from the seed having the restoring fertility gene-male gamete inhibition construct.

### Definitions

As used herein and in the appended claims, the singular forms “a”, “an”, and “the” include plural reference unless the context clearly dictates otherwise. Thus, for example, reference to “a plant” includes a plurality of such plants; reference to “a cell” includes one or more cells and equivalents thereof known to those skilled in the art, and so forth.

“Coding region” generally refers to the portion of a messenger RNA (or the corresponding portion of another nucleic acid molecule such as a DNA molecule) which encodes a protein or polypeptide. “Non-coding region” generally refers to all portions of a messenger RNA or other nucleic acid molecule that are not a coding region, including but not limited to, for example, the promoter region, 5′ untranslated region (“UTR”), 3′ UTR, intron and terminator. The terms “coding region” and “coding sequence” are used interchangeably herein. The terms “non-coding region” and “non-coding sequence” are used interchangeably herein.

“Cosuppression” generally refers to the production of sense RNA transcripts capable of suppressing the expression of the target gene or gene product. “Sense” RNA generally refers to RNA transcript that includes the mRNA and can be translated into protein within a cell or in vitro. Cosuppression constructs in plants have been previously designed by focusing on overexpression of a nucleic acid sequence having homology to a native mRNA, in the sense orientation, which results in the reduction of all RNA having homology to the overexpressed sequence (see Vaucheret et al., Plant J. 16:651-659 (1998); and Gura, Nature 404:804-808 (2000)).

The term “crossed” or “cross” or “crossing” in the context of this disclosure means the fusion of gametes via pollination to produce progeny (i.e., cells, seeds, or plants). The term encompasses both sexual crosses (the pollination of one plant by another) and selfing (self-pollination, i.e., when the pollen and ovule (or microspores and megaspores) are from the same plant or genetically identical plants).

“Expression” generally refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

The terms “full complement” and “full-length complement” are used interchangeably herein, and refer to a complement of a given nucleotide sequence, wherein the complement and the nucleotide sequence consist of the same number of nucleotides and are 100% complementary.

“Gamete” refers to a reproductive cell having the 1n set (haploid number) of chromosomes that can fuse with another gamete of the opposite sex during fertilization in organisms undergoing sexual reproduction. As used herein, a gamete in organisms undergoing asexual reproduction refers to a cell having a 2n number (an unreduced number) of chromosomes.

The term “gene” as used herein refers to a polynucleotide that is expressed by at least one of transcription and translation. An example of a gene is a nucleic acid fragment capable of being transcribed into mRNA or translated into a protein. A “gene” may or may not include a coding region or a regulatory sequence of a 5′-non coding sequence and a 3′-non coding sequence in addition to the coding region. For example, a Ms5 gene refers to a Ms5 polynucleotide that is expressed by at least one of transcription and translation.

As used herein, the term “gene locus” refers to the position of a gene on a genome. For example, Ms5 gene locus refers to the position of a Ms5 gene on genome.

The term “genome” refers to the entire complement of genetic material (genes and non-coding sequences) that is present in each cell of an organism, or virus or organelle; and/or a complete set of chromosomes inherited as a (haploid) unit from one parent.

“Heterologous” with respect to sequence means a sequence that originates from a foreign species, or, if from the same species, is substantially modified from its native form in composition and/or genomic locus by deliberate human intervention.

The term “introduced” in the context of inserting a nucleic acid into a cell,” and includes reference to the incorporation of a nucleic acid or nucleic acid fragment into a eukaryotic or prokaryotic cell where the nucleic acid may be incorporated into the genome of the cell (e.g., chromosome, plasmid, plastid or mitochondrial DNA), converted into an autonomous replicon or transiently expressed (e.g., transfected mRNA).

“Isolated” generally refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. The term also embraces recombinant polynucleotides and chemically synthesized polynucleotides.

As used herein, a “male sterile plant” is a plant that does not produce male gametes that are viable or otherwise capable of fertilization.

The term “miRNA* sequence” refers to a sequence in the miRNA precursor that is highly complementary to the miRNA sequence. The miRNA and miRNA* sequences form part of the stem region of the miRNA precursor hairpin structure.

The terms “monocot” and “monocotyledonous plant” are used interchangeably herein. A monocot of the current disclosure includes the Gramineae.

“Percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. In certain embodiments, sequence identity may be based on the Clustal V or Clustal W method of alignment. The term “about” when used herein in context with percent sequence identity means +/−1.0%.

The term “plant” refers to whole plants, plant organs, plant tissues, seeds, plant cells, seeds and progeny of the same. Plant cells include, without limitation, cells from seeds, suspension cultures, embryos, meristematic regions, callus tissue, leaves, roots, shoots, gametophytes, sporophytes, pollen and microspores. Plant parts include differentiated and undifferentiated tissues including, but not limited to roots, stems, shoots, leaves, pollen, seeds, tumor tissue and various forms of cells and culture (e.g., single cells, protoplasts, embryos, and callus tissue). The plant tissue may be in plant or in a plant organ, tissue or cell culture. The term “plant organ” refers to plant tissue or a group of tissues that constitute a morphologically and functionally distinct part of a plant.

“Polynucleotide”, “nucleic acid sequence”, “nucleotide sequence”, or “nucleic acid fragment” are used interchangeably and is a polymer of RNA or DNA that is single- or double-stranded, optionally containing synthetic, non-natural or altered nucleotide bases.

As used herein, “polynucleotide” includes reference to a deoxyribopolynucleotide, ribopolynucleotide or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

“Polypeptide”, “peptide”, “amino acid sequence” and “protein” are used interchangeably herein to refer to a polymer of amino acid residues.

“Progeny” comprises any subsequent generation of a plant.

“Promoter functional in a plant” is a promoter capable of controlling.

transcription in plant cells whether or not its origin is from a plant cell.

As used herein “promoter” includes reference to a region of DNA upstream from the start of transcription and involved in recognition and binding of RNA polymerase and other proteins to initiate transcription.

A “plant promoter” is a promoter capable of initiating transcription in plant cells.

“Recombinant” generally refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” generally refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature. The terms “recombinant DNA construct” and “recombinant construct” are used interchangeably herein.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-90% sequence identity and most preferably 100% sequence identity (i.e., complementary) with each other.

The terms “stringent conditions” or “stringent hybridization conditions” means conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence. The term “under stringent conditions” means that two sequences hybridize under moderately or highly stringent conditions. More specifically, moderately stringent conditions can be readily determined by those having ordinary skill in the art, e.g., depending on the length of DNA. The basic conditions are set forth by Sambrook et al., Molecular Cloning: A Laboratory Manual, third edition, chapters 6 and 7, Cold Spring Harbor Laboratory Press, 2001 and include the use of a prewashing solution for nitrocellulose filters 5×SSC, 0.5% SDS, 1.0 mM EDTA (pH 8.0), hybridization conditions of about 50% formamide, 2×SSC to 6×SSC at about 40-50° C. (or other similar hybridization solutions, such as Stark's solution, in about 50% formamide at about 42° C.) and washing conditions of, for example, about 40-60° C., 0.5-6×SSC, 0.1% SDS. Preferably, moderately stringent conditions include hybridization (and washing) at about 50° C. and 6×SSC. Highly stringent conditions can also be readily determined by those skilled in the art, e.g., depending on the length of DNA.

Generally, such conditions include hybridization and/or washing at higher temperature and/or lower salt concentration (such as hybridization at about 65° C., 6×SSC to 0.2×SSC, preferably 6×SSC, more preferably 2×SSC, most preferably 0.2×SSC), compared to the moderately stringent conditions. For example, highly stringent conditions may include hybridization as defined above, and washing at approximately 65-68° C., 0.2×SSC, 0.1% SDS. SSPE (1×SSPE is 0.15 M NaCl, 10 mM NaH2PO4, and 1.25 mM EDTA, pH 7.4) can be substituted for SSC (1×SSC is 0.15 M NaCl and 15 mM sodium citrate) in the hybridization and washing buffers; washing is performed for 15 minutes after hybridization is completed.

The term “substantial identity” of polynucleotide sequences means that a polynucleotide comprises a sequence that has between 50-100% sequence identity, preferably at least 50% sequence identity, preferably at least 60% sequence identity, preferably at least 70%, more preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 55-100%, preferably at least 55%, preferably at least 60%, more preferably at least 70%, 80%, 90% and most preferably at least 95%.

Another indication that nucleotide sequences are substantially identical is if two molecules hybridize to each other under stringent conditions. The degeneracy of the genetic code allows for many amino acids substitutions that lead to variety in the nucleotide sequence that code for the same amino acid, hence it is possible that the DNA sequence could code for the same polypeptide but not hybridize to each other under stringent conditions. This may occur, e.g., when a copy of a nucleic acid is created using the maximum codon degeneracy permitted by the genetic code. One indication that two nucleic acid sequences are substantially identical is that the polypeptide, which the first nucleic acid encodes, is immunologically cross reactive with the polypeptide encoded by the second nucleic acid.

The terms “substantial identity” in the context of a peptide indicates that a peptide comprises a sequence with between 55-100% sequence identity to a reference sequence preferably at least 55% sequence identity, preferably 60% preferably 70%, more preferably 80%, most preferably at least 90% or 95% sequence identity to the reference sequence over a specified comparison window. Preferably, optimal alignment is conducted using the homology alignment algorithm of Needleman and Wunsch, supra. An indication that two peptide sequences are substantially identical is that one peptide is immunologically reactive with antibodies raised against the second peptide. Thus, a peptide is substantially identical to a second peptide, for example, where the two peptides differ only by a conservative substitution. In addition, a peptide can be substantially identical to a second peptide when they differ by a non-conservative change if the epitope that the antibody recognizes is substantially identical. Peptides, which are “substantially similar” share sequences as, noted above except that residue positions, which are not identical, may differ by conservative amino acid changes.

The terms “suppress”, “suppressed”, “suppression”, “suppressing” and “silencing”, are used interchangeably herein and include lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches and the like.

“Transcription terminator”, “termination sequences”, or “terminator” refer to DNA sequences located downstream of a protein-coding sequence, including polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht, I. L., et al., Plant Cell 1:671-680 (1989). A polynucleotide sequence with “terminator activity” generally refers to a polynucleotide sequence that, when operably linked to the 3′ end of a second polynucleotide sequence that is to be expressed, is capable of terminating transcription from the second polynucleotide sequence and facilitating efficient 3′ end processing of the messenger RNA resulting in addition of poly A tail. Transcription termination is the process by which RNA synthesis by RNA polymerase is stopped and both the processed messenger RNA and the enzyme are released from the DNA template.

The term “under stringent conditions” means that two sequences hybridize under moderately or highly stringent conditions.

As used herein, the term “wheat” refers to any species of the genus Triticum, including progenitors thereof, as well as progeny thereof produced by crosses with other species. Wheat includes “hexaploid wheat” which has genome organization of AABBDD, comprised of 42 chromosomes, and “tetraploid wheat” which has genome organization of AABB, comprised of 28 chromosomes. Hexaploid wheat includes T. aestivum, T. spelta, T. mocha, T. compactum, T. sphaerococcum, T. vavilovii, and interspecies cross thereof. Tetraploid wheat includes T. durum (also referred to as durum wheat or Triticum turgidum ssp. durum), T. dicoccoides, T. dicoccum, T. polonicum, and interspecies cross thereof. In addition, the term “wheat” includes possible progenitors of hexaploid or tetraploid Triticum sp. such as T. uartu, T. monococcum or T. boeoticum for the A genome, Aegilops speltoides for the B genome, and T. tauschii (also known as Aegilops squarrosa or Aegilops tauschii) for the D genome. A wheat cultivar for use in the present disclosure may belong to, but is not limited to, any of the above-listed species. Also encompassed are plants that are produced by conventional techniques using Triticum sp. as a parent in a sexual cross with a non-Triticum species, such as rye (Secale cereale), including but not limited to Triticale. In some embodiments, the wheat plant is suitable for commercial production of grain, such as commercial varieties of hexaploid wheat or durum wheat, having suitable agronomic characteristics which are known to those skilled in the art.

The meaning of abbreviations is as follows: “sec” means second(s), “min” means minute(s), “h” means hour(s), “d” means day(s), “μL” means microliter(s), “mL” means milliliter(s), “L” means liter(s), “UM” means micromolar, “mM” means millimolar, “M” means molar, “mmol” means millimole(s), “μmole” mean micromole(s), “g” means gram(s), “μg” means microgram(s), “ng” means nanogram(s), “U” means unit(s), “bp” means base pair(s) and “kb” means kilobase(s).

Also, as described herein, for each example or embodiment that cites a guide RNA, a similar guide polynucleotide can be designed wherein the guide polynucleotide does not solely comprise ribonucleic acids but wherein the guide polynucleotide comprises a combination of RNA-DNA molecules or solely comprises DNA molecules.

### EXAMPLES

In the following Examples, unless otherwise stated, parts and percentages are by weight and degrees are Celsius. It should be understood that these Examples, while indicating embodiments of the disclosure, are given by way of illustration only. From the above discussion and these Examples, one skilled in the art can make various changes and modifications of the disclosure to adapt it to various usages and conditions. Such modifications are also intended to fall within the scope of the appended claims.

### Example 1: Expression Cassettes for Guide RNA/Cas Endonuclease Based Genome Modification in Wheat Plants

The gRNA expression cassette consisted of the wheat U6 promoter and the gRNA scaffold, both of which are described in Shan et al. 2013, Nature Biotechnology 31:686-688. See, SEQ ID NOs: 202 and 204 respectively.

The Cas9 expression cassette consisted of the Zea mays Ubiquitin 1 promoter (described in Christensen et al. 1992, Plant Molecular Biology 18:675-689), the rice codon-optimised Cas9 gene (described in Shan et al. 2013, Nature Biotechnology 31:686-688), and the Sorghum bicolor actin terminator. See, SEQ ID NOs: 205-207 respectively. The selection cassette consisted of the Zea mays Ubiquitin 1 promoter with modified first intron, the intron-containing bar gene and the wheat rbcS terminator (described in Sasanuma 2001, Molecular Genetics and Genomics 265:161-171). See, SEQ ID NOs: 212 and 203-respectively.

### Example 2: Wheat Transformation

Agrobacterium-mediated transformation of wheat cv. Fielder and cv. Gladius was carried out as described (Ishida et al. 2015, Methods in Molecular Biology 1223:189-198), with minor modifications. Briefly, immatures embryos were isolated from spikes harvested at 14 days post-anthesis. Isolated embryos were transferred to WLS-liq solution, centrifuged at 16,000 g for 10 mins, incubated in WLS-inf solution containing Agrobacterium (AGL1) for 5 mins, and then transferred to WLS-AS media for two days of co-cultivation. After co-cultivation, embryo axes were removed, and then scutella were transferred to WLS-Res media for five days of resting culture. After the resting culture, scutella were transferred to WLS-P5 callus induction media (selection with 5 mg/L phosphinothricin) for two weeks, followed by WLS-P10 callus induction media (selection with 10 mg/L phosphinothricin) for three weeks. Calli were then transferred to LSZ-P5 regeneration media for two weeks under a cycle of 12 hours dark/12 hours light (˜70 μmol/m2/s). Regenerants were transferred to LSF-P5 rooting media for two weeks, before being transferred to potted soil in the greenhouse. Timentin was substituted for cefotaxime in all tissue culture media.

### Example 3: TaMs1 Mutation in B Genome with CRISPR-Cas9 Results in Male Sterile Wheat

This example shows that homozygous TaMs1 knockout mutant plants derived from CRISPR-Cas9 induced mutations in the B genome (chromosome 4BS) exhibit a male sterile phenotype. The T0 line GL353-119 was a biallelic heterozygous mutant on 4BS with a +1 insertion in one allele, and a −3 deletion in the other allele. Both mutations were located precisely at the canonical Cas9 cut site for gRNA LTPG1-2 (SEQ ID NO:82). GL353-119 was partially sterile. GL353-119 was crossed with wildtype Gladius to produce +1/WT and −3/WT seeds (T1 generation). One of the +1/WT seeds that lacked DsRed expression was planted and grown to maturity to produce T2 seeds. Thirty of these T2 seeds were planted out, and the seedlings were genotyped. Of the 30 seedlings, four were +1/+1, 18 were +1/WT, and eight were WT/WT. The thirty T2 seedlings were grown to maturity. All +1/+1 mutants were fully sterile, whereas all +1/WT and WT/WT plants were fully fertile.

### Example 4: Phenotypic Assessment Facilitating Mapping of Ms5_{FS20}

Phenotyping for genetic male sterility was performed by quantitative and/or qualitative methods. For both methods at least 3 spikes per plant were securely covered with sealed white paper bags prior to anthesis and were then used for fertility assessment. A quantitative fertility score was determined by counting the number of florets per spike and the number of seeds per spike and expressing the score as the number of seeds per floret formed. A qualitative assessment was made by visual examination of the spikes for seed set and evidence of anther dehiscence. Anthers of ms5_{FS20} plants do not dehisce and florets of heads bagged prior to anthesis do not set seed and are deemed male sterile, while spikes of Ms5 plants show high levels of dehisced anthers and a high proportion of florets with seed and therefore deemed male fertile.

### Example 5: Genetic Mapping of Ms5

This example demonstrates that by using recombinant mapping populations of wild-type and male-sterile wheat, the causative locus for the male-sterile phenotype of wheat ms5_{FS20} can be mapped to a 0.012 cM region proximal on the long arm of chromosome 3 of the A genome. Populations ms5_{FS20}×H45 and ms5_{FS20}×Excalibur were selected for genetic mapping of Ms5 because inheritance of sterility in these populations was mono-factorial and bi-factorial respectively. Fine mapping was performed using ms5_{FS20}×H45 populations because mono-factorial inheritance provides a greater proportion of informative lines per number of lines genotyped than populations where inheritance is bi-factorial. y A male sterile (msms) wheat, var. Chris, carrying the FS20 mutant gene (also referred to as FS-20, ms5 and ms5_{FS20}) was crossed to plants of cvs. H45 and Excalibur to create F₂ mapping populations. Initial mapping to establish a broad interval spanning the Ms5 locus was undertaken in the ms5_{FS20}×Excalibur population. Sequences that were genetically positioned across a region covering the proximal region of 3AS and most of 3AL were targeted for marker development and were identified based either on synteny with wheat chromosome 3B (Choulet et al. 2014. Science 345 (6194): 1249721), barley chromosome 3H (Mayer et al. 2012. Nature 491 (7426): 711-716), rice chromosome 1 (Kawahara et al., 2013 Rice 6:4) or from a 90K consensus wheat single-nucleotide polymorphism (SNP) map (Wang et al., 2014 Plant Biotechnology 12:787-796). For example, corresponding wheat sequence contigs from reference syntenic sequences (e.g. rice gene LOC_Os01g42210, for which SEQ ID NO:48 represents a reference sequence) were identified by BLASTn to chromosome 3A-derived IWGSC (International Wheat Genome Sequencing Consortium) survey sequence assemblies (Mayer et al., 2014 Science 345 (6194): 1251788) or to Chinese Spring TGACv1 scaffolds (Clavijo et al., 2017 Genome Res 27:885-896) or to Synthetic W7984 scaffolds (Chapman et al. 2014 Genome Biol 16:26). Several methods were used to identify SNP-containing wheat sequences; direct comparison of Illumina HiSeq genomic sequences of 40 homozygous ms5_{FS20} individuals and 20 homozygous Ms5_{FS20} individuals, mapping of RNAseq reads from a homozygous fertile Ms5_{FS20} plant against 454 sequences of cv. Excalibur (ref BioPlatforms Australia), retrieval and examination of ms5_{FS20} and Ms5_{FS20} promoter sequences of anther transcripts which had been identified by RNAseq to be differentially expressed between ms5_{FS20} and Ms5_{FS20} lines. Identified SNPs were selected for marker design based on location of homeologous 3B sequences, location of orthologous 3H sequences or location of orthologous rice sequences. SNPs were further prioritized based on SNP type, with C/G to T/A transitions preferred, and on rarity of the ms5_{FS20} base when compared to sequences of homeologues and other wheat cultivars (ref BioPlatforms Australia). Identified SNPs were targeted for High Resolution Melting (HRM) marker development, Kompetitive Allele Specific PCR (KASP) marker development or Cleaved Amplified Polymorphic Sequence (CAPS) marker development. CAPS markers were developed using the NEBcutter v2.0 tool (Vincze et al., 2003 Nucleic Acids Res 31 (13): 3688-3691).

A set of the developed markers was used to genotype 2,300 ms5_{FS20}×Excalibur F₂ and F₃ plants, providing a subset of 325 plants which were predicted to be homozygous ms5_{FS20} or recombinant in the region of ms5_{FS20} and which were grown for phenotyping. Markers flanking ms5_{FS20} were experimentally determined by linkage analysis of 106 plants from the subset that showed complete sterility. In contrast to the report of Klindworth et al. (2002), the analysis showed that the region containing Ms5 is highly proximal to the 3A centromere, locating between the short arm marker MP0070 (SEQ ID: 113) and the long arm marker MP0061 (SEQ ID NO: 112). Markers MP0070 and MP0061 correspond to 3A-derived IWGSC sequence contigs Icl|3AS_3345038 and Icl|3AL_4288243 respectively (Mayer et al., 2014 Science 345 (6194): 1251788). This region was determined to approximately cover a genetic distance of 0.77 cM on the 90K consensus map (Wang et al., 2014 Plant Biotechnology 12:787-796). Inheritance of sterility in this population was determined to be controlled by homeologous loci on chromosomes 3A and 3D, with fertility levels comparable to those of wild-type plants observed in ms5_{FS20}/ms5_{FS20} genotypes which were homozygous for Excalibur alleles in the 3D region corresponding to the region on 3A shown to contain ms5_{FS20}.

Fine mapping of ms5_{FS20} was performed using 743 ms5_{FS20}×H45. F₂ individuals which were screened with markers identified to be flanking the Ms5 region on chromosome 3A and polymorphic between ms5_{FS20} and cv. H45. F₂ individuals were assessed phenotypically for male sterility using procedures described elsewhere. 16 recombinant lines were identified, and the Ms5 locus was located to a 0.13 cM interval between the KASP markers MP0091 (SEQ ID NO: 121) and MP0192 (SEQ ID NO: 122). KASP markers MP0091 and MP0192 were designated to 3AL-derived IWGSC sequence contigs Icl|3AL_4321937 and Icl|3AL_4455020, respectively (Mayer et al., 2014 Science 345 (6194): 1251788). Markers were then developed in the region between markers MP0091 and MP0192 and tested for their association with the male sterility phenotype. A total of 7721 F₃ and F₄ ms5_{FS20}×H45 individuals, derived from lines that were known to be heterozygous in the region of Ms5, were screened and 15 recombinants were identified, narrowing the Ms5-containing region to an area bounded by markers MP0156 (SEQ ID NO:117) and MP0192 (SEQ ID NO:122). Markers MP0156 and MP0192 correspond to 3AL-derived IWGSC sequence contigs Icl|3AL_4306089 and Icl|3AL_4455020 respectively (Mayer et al., 2014 Science 345 (6194): 1251788) and define a 0.012 cM region in the cross ms5_{FS20}×H45.

### Example 6: Genetic Mapping of a 3D Locus Restoring Fertility to ms5_{FS20}

This example demonstrates that by using recombinant mapping populations of wild-type and male-sterile wheat, a locus capable of complementing the male-sterile phenotype of wheat ms5_{FS20} can be mapped to a 1.19 cM region on chromosome 3 of the D genome which is syntenous with the location of ms5_{FS20} on chromosome 3A.

Fertility assessment in an unbiased set of 80 ms5_{FS20}×Excalibur F₂ lines found three levels of fertility; high fertility, partial fertility and complete sterility. High or partial fertility was present in 74 lines (92.5%) and complete sterility in 6 lines (7.5%), consistent with bi-factorial control of fertility in Excalibur (Fishers Exact Test, 2-tail p=1). Similarly, fertility assessment of an unbiased set of 209 ms5_{FS20}×Gladius F₂ lines found three levels of fertility; high fertility, partial fertility and sterility. High or partial fertility was present in 191 lines (91.4%) and sterility in 18 lines (8.6%), consistent with bi-factorial control of fertility in Gladius (Fisher's Exact Test, 2-tail p=0.4558).

The subset of 325 ms5_{FS20}×Excalibur F₂ and F₃ plants described above was used to investigate full and partial fertility restoration that was observed in a proportion of lines which were expected to be homozygous for ms5_{FS20} based on flanking marker genotype. Limiting the linkage analysis to 84 such lines which were observed to be highly fertile identified centromeric-proximal markers closely linked to a fertility restoration locus on chromosome 3D.

Fine mapping was performed using two populations; 209 ms5_{FS20}×Gladius F₂ individuals and 93 ms5_{FS20}×RAC875 F₂ individuals. The populations were screened with markers identified to be flanking the Ms5 region on chromosome 3D and polymorphic between ms5_{FS20} and cv. Gladius or between ms5_{FS20} and cv. RAC875. F₂ individuals were assessed phenotypically for male sterility using procedures described elsewhere herein (see Example 4.). In the ms5_{FS20}×Gladius population 5 recombinant lines were identified and the fertility-restoring locus was located to a 1.19 cM interval between the KASP markers MP0216 (SEQ ID NO:131) and MP0215 (SEQ ID NO:130). KASP markers MP0216 and MP0215 were designated to 3DL-derived IWGSC sequence contigs Icl|3DL_6894520 and Icl|3DL_6852770, respectively (Mayer et al., 2014 Science 345 (6194): 1251788). In the ms5_{FS20}×RAC875 population 4 recombinant lines were identified and the fertility-restoring locus was located to a 2.15 cM interval between the KASP markers MP0211 (SEQ ID NO: 128) and MP0131 (SEQ ID NO: 127). KASP markers MP0211 and MP0131 were designated to 3DL-derived IWGSC sequence contigs Icl|3DL_6867260 and Icl|3DL_6953108, respectively (Mayer et al., 2014 Science 345 (6194): 1251788). Combining information from all three populations positioned the 3D fertility-restoring locus between markers MP0211 and MP0215.

### Example 7: Identification of Candidate Ms5 Gene and Candidate 3D Fertility Gene

Comparison of marker order across the Ms5 region in 3A obtained by genetic mapping in the populations described above with that predicted by the then current 3B and 3H pseudomolecules showed limited agreement. Conversely, comparison to gene order in the rice Nipponbare RGAP 7 assembly (reference goes here) indicated a high degree of collinearity and therefore a 0.75 Mb interval of the rice genome from LOC_Os01g41030, which corresponds to MP0156, to LOC_Os01g42294, which corresponds to MP0192, was examined for Ms5 candidates.

Table 4 lists the 122 annotated rice genes within the interval LOC_Os01g41030 to LOC_Os01g42294 and their putative peptide function.

TABLE 4

| locus name | functional annotation |
|---|---|
| LOC_Os01g41030 | CDS ribosomal protein L25, putative |
| LOC_Os01g41040 | CDS SCF apoptosis response protein, putative |
| LOC_Os01g41050 | CDS sulfate transporter, putative |
| LOC_Os01g41060 | CDS retrotransposon protein, putative, unclassified |
| LOC_Os01g41070 | CDS retrotransposon protein, putative, unclassified |
| LOC_Os01g41080 | CDS retrotransposon protein, putative, unclassified |
| LOC_Os01g41090 | CDS retrotransposon protein, putative, unclassified |
| LOC_Os01g41100 | CDS retrotransposon protein, putative, unclassified |
| LOC_Os01g41110 | CDS expressed protein |
| LOC_Os01g41120 | CDS retrotransposon protein, putative, Ty3-gypsy |
| | subclass |
| LOC_Os01g41140 | CDS THION18 - Plant thionin family protein |
| | precursor |
| LOC_Os01g41145 | CDS retrotransposon protein, putative, unclassified |
| LOC_Os01g41160 | CDS FAD dependent oxidoreductase domain |
| | containing protein |
| LOC_Os01g41170 | CDS THION27 - Plant thionin family protein |
| | precursor |
| LOC_Os01g41180 | CDS THION19 - Plant thionin family protein |
| | precursor, putative |
| LOC_Os01g41190 | CDS glycine-rich protein, putative |
| LOC_Os01g41200 | CDS heavy metal-associated domain containing |
| | protein |
| LOC_Os01g41210 | CDS transposon protein, putative, unclassified |
| LOC_Os01g41220 | CDS DUF538 domain containing protein, putative |
| LOC_Os01g41230 | CDS hypothetical protein |
| LOC_Os01g41240 | CDS hydrolase, alpha/beta fold family domain |
| | containing protein |
| LOC_Os01g41250 | CDS OsFBX17 - F-box domain containing protein |
| LOC_Os01g41260 | CDS OsFBD2 - F-box and FBD domain containing |
| | protein |
| LOC_Os01g41270 | CDS OsFBD3 - F-box and FBD domain containing |
| | protein |
| LOC_Os01g41280 | CDS OsFBD4 - F-box and FBD domain containing |
| | protein |
| LOC_Os01g41290 | CDS OsFBD5 - F-box and FBD domain containing |
| | protein |
| LOC_Os01g41300 | CDS expressed protein |
| LOC_Os01g41310 | CDS OsFBX18 - F-box domain containing protein |
| LOC_Os01g41320 | CDS expressed protein |
| LOC_Os01g41330 | CDS hypothetical protein |
| LOC_Os01g41340 | CDS OsFBL1 - F-box domain and LRR containing |
| | protein |
| LOC_Os01g41350 | CDS expressed protein |
| LOC_Os01g41360 | CDS expressed protein |
| LOC_Os01g41370 | CDS FBD domain containing protein, putative |
| LOC_Os01g41390 | CDS retrotransposon, putative, centromere-specific |
| LOC_Os01g41400 | CDS transmembrane amino acid transporter |
| | protein, putative |
| LOC_Os01g41410 | CDS expressed protein |
| LOC_Os01g41420 | CDS transmembrane amino acid transporter |
| | protein, putative |
| LOC_Os01g41430 | CDS UDP-glucoronosyl and UDP-glucosyl |
| | transferase, putative |
| LOC_Os01g41440 | CDS retrotransposon protein, putative, unclassified |
| LOC_Os01g41450 | CDS UDP-glucoronosyl and UDP-glucosyl |
| | transferase domain containing protein |
| LOC_Os01g41460 | CDS retrotransposon protein, putative, Ty3-gypsy |
| | subclass |
| LOC_Os01g41470 | CDS retrotransposon protein, putative, Ty3-gypsy |
| | subclass |
| LOC_Os01g41480 | CDS retrotransposon protein, putative, Ty3-gypsy |
| | subclass |
| LOC_Os01g41490 | CDS retrotransposon protein, putative, Ty3-gypsy |
| | subclass |
| LOC_Os01g41500 | CDS retrotransposon protein, putative, unclassified |
| LOC_Os01g41510 | CDS calcineurin B, putative |
| LOC_Os01g41516 | CDS retrotransposon protein, putative, Ty1-copia |
| | subclass |
| LOC_Os01g41522 | CDS retrotransposon protein, putative, unclassified |
| LOC_Os01g41530 | CDS OsFBL2 - F-box domain and LRR containing |
| | protein |
| LOC_Os01g41540 | CDS hypothetical protein |
| LOC_Os01g41550 | CDS aspartic proteinase, putative |
| LOC_Os01g41560 | CDS hypothetical protein |
| LOC_Os01g41565 | CDS ATP-binding domain-containing protein, |
| | putative |
| LOC_Os01g41580 | CDS expressed protein |
| LOC_Os01g41590 | CDS transposon protein, putative, unclassified |
| LOC_Os01g41600 | CDS Sad1/UNC-like C-terminal domain |
| | containing protein, putative |
| LOC_Os01g41610 | CDS mitochondrial ATP synthase g subunit |
| | family protein, putative |
| LOC_Os01g41620 | CDS transposon protein, putative, unclassified |
| LOC_Os01g41630 | CDS serine/threonine protein phosphatase 2A |
| | 55 kDa regulatory subunit B, putative |
| LOC_Os01g41640 | CDS expressed protein |
| LOC_Os01g41650 | CDS pentatricopeptide, putative |
| LOC_Os01g41660 | CDS phosphoethanolamine/phosphocholine |
| | phosphatase, putative |
| LOC_Os01g41670 | CDS G-patch domain containing protein, putative |
| LOC_Os01g41680 | CDS retrotransposon protein, putative, unclassified |
| LOC_Os01g41690 | CDS transposon protein, putative, unclassified |
| LOC_Os01g41700 | CDS transposon protein, putative, unclassified |
| LOC_Os01g41710 | CDS chlorophyll A-B binding protein, putative |
| LOC_Os01g41720 | CDS expressed protein |
| LOC_Os01g41730 | CDS serine/threonine-protein kinase, putative |
| LOC_Os01g41740 | CDS expressed protein |
| LOC_Os01g41750 | CDS expressed protein |
| LOC_Os01g41760 | CDS expressed protein |
| LOC_Os01g41770 | CDS leucine rich repeat protein, putative |
| LOC_Os01g41780 | CDS expressed protein |
| LOC_Os01g41790 | CDS expressed protein |
| LOC_Os01g41800 | CDS cytochrome P450, putative |
| LOC_Os01g41810 | CDS cytochrome P450 72A1, putative |
| LOC_Os01g41820 | CDS cytochrome P450 72A1, putative |
| LOC_Os01g41834 | CDS chalcone synthase, putative |
| LOC_Os01g41850 | CDS transposon protein, putative, unclassified |
| LOC_Os01g41860 | CDS hypothetical protein |
| LOC_Os01g41870 | CDS protein kinase, putative |
| LOC_Os01g41880 | CDS hyaluronan/mRNA binding family domain |
| | containing protein |
| LOC_Os01g41890 | CDS MLA1, putative |
| LOC_Os01g41900 | CDS Myb transcription factor, putative |
| LOC_Os01g41910 | CDS receptor-like protein kinase 5 precursor, |
| | putative |
| LOC_Os01g41920 | CDS expressed protein |
| LOC_Os01g41930 | CDS leucine rich repeat protein, putative |
| LOC_Os01g41950 | CDS expressed protein |
| LOC_Os01g41960 | CDS retrotransposon protein, putative, unclassified |
| LOC_Os01g41970 | CDS expressed protein |
| LOC_Os01g41980 | CDS retrotransposon protein, putative, unclassified |
| LOC_Os01g41990 | CDS OsCML12 - Calmodulin-related calcium |
| | sensor protein |
| LOC_Os01g42000 | CDS skin secretory protein xP2 precursor, putative |
| LOC_Os01g42010 | CDS expressed protein |
| LOC_Os01g42020 | CDS retrotransposon protein, putative, LINE |
| | subclass |
| LOC_Os01g42024 | CDS expressed protein |
| LOC_Os01g42030 | CDS mitochondrial chaperone BCS1, putative |
| LOC_Os01g42040 | CDS ubiquitin-conjugating enzyme, putative |
| LOC_Os01g42050 | CDS DNL zinc finger domain containing protein, |
| | putative |
| LOC_Os01g42060 | CDS expressed protein |
| LOC_Os01g42070 | CDS kinesin motor domain containing protein, |
| | putative |
| LOC_Os01g42080 | CDS zinc ion binding protein, putative |
| LOC_Os01g42090 | CDS nodulin MtN3 family protein, putative |
| LOC_Os01g42100 | CDS expressed protein |
| LOC_Os01g42110 | CDS nodulin MtN3 family protein, putative |
| LOC_Os01g42120 | CDS expressed protein |
| LOC_Os01g42130 | CDS expressed protein |
| LOC_Os01g42140 | CDS expressed protein |
| LOC_Os01g42150 | CDS MEGL13 - Maternally expressed gene MEG |
| | family protein precursor |
| LOC_Os01g42160 | CDS MEGL14 - Maternally expressed gene MEG |
| | family protein precursor, putative |
| LOC_Os01g42170 | CDS zinc knuckle family protein |
| LOC_Os01g42190 | CDS heat shock protein DnaJ, putative |
| LOC_Os01g42200 | CDS expressed protein |
| LOC_Os01g42210 | CDS LTPL47 - Protease inhibitor/seed storage/LTP |
| | family protein precursor, putative |
| LOC_Os01g42220 | CDS expressed protein |
| LOC_Os01g42234 | CDS amino acid permease family protein, putative |
| LOC_Os01g42260 | CDS transcriptional corepressor LEUNIG, putative |
| LOC_Os01g42270 | CDS transcriptional corepressor LEUNIG, putative |
| LOC_Os01g42280 | CDS pentatricopeptide, putative |
| LOC_Os01g42294 | CDS inactive receptor kinase At2g26730 precursor, |
| | putative |

Among retrieved wheat sequences corresponding to the 122 annotated loci in the rice interval, 10 contained SNPs between ms5_{FS20} and cv. H45. One identified SNP was predicted to be in the coding sequence of the wheat orthologue of LOC_Os01g42210, a polypeptide with similarity to non-specific lipid transfer protein (nsLTP) (Edstam et al., 2014 Physiologia Plantarum doi: 10.1111/ppl.12156). This particular sequence is predicted to encode a glycosylphosphatidylinositol (GPI)-anchored nsLTP (LTPG) polypeptide (SEQ ID NO:19 is the amino acid sequence of the encoded protein). A functionally related sequence, TaLTPG1 (syn. TaMS1), was determined to have a crucial role in anther development (Tucker et al., 2017, Nature Communications 8 (869): 1-10), with mutated forms underlying the male-sterile phenotypes of ms1d, ms1e and ms1f. Therefore the identified 3A sequence has been named TaLTPG2-3A. Examination of retrieved sequences of homeologous loci TaLTPG2-3B and TaLTPG2-3D found two allelic forms for each homeolocus. Both alleles of TaLTPG2-3B were predicted to encode non-functional LTPG-type proteins as a result of coding sequence deletions. Genetic mapping of TaLTPG2-3D in ms5_{FS20}×Excalibur, ms5_{FS20}×Gladius and ms5_{FS20}×RAC875 located it within the determined critical fertility-restoring interval in each population. One allele of TaLTPG2-3D was predicted to encode a functional LTPG-type protein and this allelic form was found in the cultivars Excalibur, Gladius, RAC875 and Chinese Spring. A second allele of TaLTPG2-3D was predicted to encode a non-functional LTPG-type protein as a result of an exonic single base insertion at position 76-77 (−/C) of the genomic sequence of the functional form (SEQ ID NO: 28). The non-functional allelic form was found in cultivars Chris and H45 which do not contain sequences capable of complementing the ms5_{FS20} phenotype. Agreement between allelic form, observed phenotype and trait inheritance pattern suggested TaLTPG2-3A as a likely candidate for Ms5.

### Example 8: Isolation and Sequence of Wheat Mutant ms5_{FS20} Allele

Full-length coding sequences of TaLTPG2-3A from chromosome 3AL were PCR amplified from genomic DNAs isolated from male sterile homozygous Ethyl methanesulfonate (EMS)-induced mutant ms5_{FS20} (Klindworth et al., 2002 Crop Sci. 42:1447-1450) and wild-type (Ms5) male fertile genotypes (cultivar Chris). Both strands of PCR amplicons were sequenced using standard Sanger sequencing techniques for GC-rich products. The Sanger sequencing chromatograms revealed a SNP between the ms5_{FS20} mutant allele and the wild-type sequence. Sequence analysis predicts that protein function is disrupted for this mutant.

ms5_{FS20} exhibits a SNP at position 101 (G101A) when compared to wild-type Ms5 genomic DNA sequence (SEQ ID NO:16). This SNP is predicted to convert a conserved Cysteine to a Tyrosine (C34Y) within the encoded wild-type Ms5 polypeptide (SEQ ID NO:19). This amino acid change is predicted to disrupt the tertiary conformation of the mature protein mediated by a putative di-sulfide bridge.

### Example 9: Sequences of Wheat Ms5 Homeologue Alleles

Sequences for the 3B and 3D Ms5 homeologues were retrieved for cultivars Chinese Spring, Chris, Excalibur, Gladius, H45, RAC875 and Synthetic W7984. Sequences for each genome were compared by alignment to detect variant alleles.

Relative to the wildtype form of Ms5, the TaLTPG2-3B allele in cultivar Chinese Spring (SEQ ID NO:21) contains a 1 bp deletion at position 140 of the reference sequence (SEQ ID NO:16), predicted to be in exon 1 and to cause a frameshift, resulting in translation to a polypeptide with no similarity to proteins of known function (SEQ ID NO:23). Relative to the wildtype form of Ms5, the TaLTPG2-3B allele in cultivar Synthetic W7984 (SEQ ID NO:199) contains a large deletion beginning within exon 1 and extending into intron 1, resulting in a shortened predicted polypeptide comprising 43 residues (SEQ ID NO: 26), the first 31 of which show homology to Ms5.

The TaLTPG2-3D alleles in cultivars Excalibur, Gladius and RAC875 and Synthetic W7984 encode identical polypeptide sequences (SEQ ID NO:34) and have high homology to the wildtype form of Ms5. The TaLTPG2-3D alleles in cultivars Chris and H45 contain an indel at position 77 of the reference TaLTPG2-3D coding sequence (SEQ ID NO:25). This indel causes a frame shift predicted to generate a non-functional truncated polypeptide comprising 141 amino acids (SEQ ID NO: 33), the first 26 of which show homology to Ms5.

### Example 10: Markers in the Ms5 Region and their Use in Identifying and Selecting Wheat Plants Containing Ms5 Mutations

The Ms5 gene was found to be tightly linked to markers MP0156, MP0179, MP0182, MP0190, MP0191, MP0192, MP0201, and MP0090 that are located in the Ms5 region. See SEQ ID NOS: 115, 117-123. The fertility restoration locus on chromosome 3D was found to be tightly linked to markers MP0126, MP0212, MP0127, MP0215 and MP0130 that are located in the Ms5 homeologous region. See SEQ ID NOS: 124-126 and 129-130. Because the male sterility trait is controlled by two nuclear recessive genes, all crosses between male sterile mutants and wild type pollinators will result in 100% male fertile F₁ progenies (Ms5ms5), whereas F₂ and BC₁ progenies will segregate for this trait. It is desirable to determine the genotypes of the progenies, and as such, plants can be evaluated for the presence of the mutation itself, or alternatively, for one or more alleles that are linked to and associated with the mutation in the Ms5 gene (i.e. in linkage disequilibrium with the mutation). For example, one or more alleles at 3A markers MP0156, MP0179, MP0182, MP0190, MP0191, MP0192, MP0201, and MP0090 may be detected to determine if a plant has an Ms5 mutation in the homozygous or heterozygous state. Likewise, one or more alleles at 3D markers MP0126, MP0212, MP0127, MP0215 and MP0130 may be detected to determine if a plant carries a non-functional allele of TaLTPG2-3D in the homozygous or heterozygous state. In the case of ms5_{FS20}, the mutations arose in the Chris variety; therefore, alleles of Chris located in the vicinity of the Ms5 gene are in linkage disequilibrium with the causal mutation and hence can be evaluated for presence or absence in order to determine if ms5_{FS20} is present. Similarly alleles of Chris located in the vicinity of TaLTPG2-3D are in linkage disequilibrium with the genetic background which permits observation of ms5_{FS20} male sterility. Through marker assisted selection, a plant breeder will be able to follow the presence of the male sterility trait through controlled crosses to obtain, when desired, a new plant containing both a non-functional 3D allele and an Ms5 mutation in either the homozygous or heterozygous state, thereby maintaining the Ms5 mutation. A plant breeder can also utilize markers in the Ms5 and TaLTPG2-3D regions to produce mutant male sterile seed parents that would be used as female, i.e. plants that need pollination by a pollen donor plant, to produce seeds of commercial interest or to produce F₁ hybrids that contain an Ms5 mutation in the heterozygous state.

### Example 11: Restoring Male Fertility to Wheat Ms5 Homozygous Recessive Plants by Expressing a Transformed Copy of an Ms5 Gene or Ortholog

In the previous example, single-nucleotide sequence differences were detected within regions of DNA that correspond to the Ms5 candidate gene from ms5_{FS20} plants. In this example, various strategies are described for restoring male fertility to homozygous recessive ms5 plants. Male-sterile wheat plants containing an ms5 mutation or deletion are restored to male fertility when transformed with a DNA vector containing a functional copy of an Ms5 gene. This demonstrates that the sequence changes within, or deletions of, the candidate Ms5 gene are the causal effect of the male-sterile phenotype.

Although wheat is an allohexaploid containing three related genomes (ABD) with similar gene content, it behaves as a diploid during meiosis. Often the related wheat genomes contain homeologous genes that have similar gene structure and function, requiring triple mutants to result in a loss-of-function phenotype. The wheat male sterility phenotype observed in the ms5_{FS20} mutant segregates at a 3:1 ratio of fertile to sterile plants if homozygous for a non-functional TaLTPG2-3D allele. This indicates that in this mutant, in selected genetic backgrounds, a single recessive locus in the homozygous condition induces a male sterility phenotype and that this locus segregates according to the laws of Mendelian inheritance. The observation of some functional redundancy with the 3D, but not the 3B, Ms5 homologue indicates that there has been partial divergence of function among the copies of this gene. Marker development and assessment has shown that the ms5 locus, in selected genetic backgrounds, segregates at a 1:2:1 ratio of homozygous wild type to heterozygous to homozygous mutant. The correlation of phenotypic and genotypic data supports the Mendelian inheritance of the ms5 mutation.

The Mendelian nature of the ms5 mutation will facilitate a simple introgression of a male sterility trait into different genetic backgrounds.

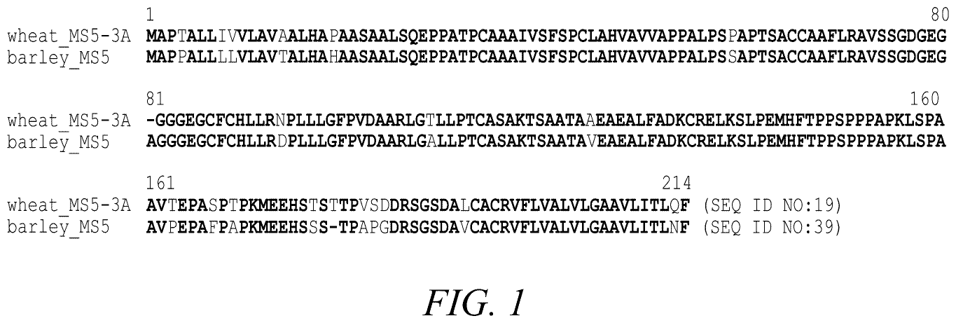

One strategy to restore male fertility to ms5 plants is to express a gene or genes that can overcome the loss of function or activity resulting from Ms5 mutation or deletion. A gene from wheat, or from another plant species, having identical or similar function to Ms5 is used to restore gene activity in transformed wheat plants. For example, as shown in FIG. 1, a gene from barley encodes a protein with high amino acid sequence similarity to the wheat Ms5 gene product, with approximately 90% sequence identity. The barley gene present within SEQ ID NO:36 is introduced into wheat ms5 mutant plants which are additionally homozygous for a non-functional TaLTPG2-3D allele to restore male fertility. This barley gene may be expressed using its native promoter (see SEQ ID NO:37, nucleotides 1-2000) or a non-native promoter, such as a tissue-preferred, constitutive or conditional promoter, to restore male fertility. Other monocot or dicot plants, or hybrid combinations thereof, can also serve as sources of a complementing gene and promoter to restore male fertility to ms5 mutant male-sterile wheat plants.

In another strategy, the wild-type wheat Ms5 gene or a variant (see, for example, SEQ ID NO:16-18, 21, 23-24) is used to restore male fertility to homozygous recessive ms5 plants which are additionally homozygous for a non-functional TaLTPG2-3D allele. The variant Ms5 gene comprises alteration of one or more DNA restriction sites to allow compatibility with DNA vectors used for plant transformation. See, for example, SEQ ID NO:198, which comprises nucleotide changes introduced at positions 1007 and 1584 to facilitate vector construction. The Ms5 gene is introduced into ms5 plants by known plant transformation methods to produce plants containing stably integrated versions of the Ms5 gene for fertility complementation. As an alternative to using the native Ms5 promoter (SEQ ID NO: 17, 22, or 30), a promoter variant (for example see SEQ ID NO:198), or other plant, such as SEQ ID NO:37, 42, 47, or 52, or non-plant constitutive, conditional or tissue-preferred promoter is used to express a wild-type or variant version of the Ms5 gene or cDNA for the purpose of restoring male fertility to homozygous recessive ms5 wheat plants. The gene and promoter may be from one source species or from a combination of source species. In some examples, the promoter is a Ms5 promoter from wheat, rice, barley or brachypodium. The genomic Ms5 sequence 3′ to the translational stop codon comprises a functional terminator region; see, for example, SEQ ID NO: 20, 27, 35, 40, 45, 50, or 55.

### Constructs and Transformation

To restore the fertility of ms5_{FS20}/ms5_{FS20} homozygous mutants, the wheat Ms5 gene under control of the native wheat Ms5 promoter and terminator was linked to a Bar gene under control of the maize ubiquitin promoter (see, e.g., SEQ ID NO: 205) and also carrying a Rbcs terminator sequence (TaMs5-UbiBar). This construct was transformed directly into wheat embryos harvested from Ms5/ms5_{FS20} heterozygous plants that were additionally homozygous for a non-functional TaLTPG2-3D allele through Agrobacterium-mediated transformation methods as referenced elsewhere herein. Several independent T-DNA insertion events containing TaMs5-UbiBar were obtained for construct evaluation in ms5_{FS20} plants.

### T0 Plant Generation and Analysis

T0 wheat plants containing one or more copies of the TaMs5-UbiBar cassette were identified and genotyped as homozygous or heterozygous for the ms5_{FS20} mutation. Selfed seed from these individual plants was counted as a qualitative measure of male fertility. As shown in Table 5, no seed set was observed in ms5_{FS20}/ms5_{FS20} homozygous plants lacking the TaMs5-UbiBar cassette. In contrast, seed set was observed when ms5_{FS20}/ms5_{FS20} homozygous plants contained a transformed copy of the TaMs5-UbiBar cassette. These results demonstrate that the transformed copy of TaMs5 was functional and able to restore fertility to ms5_{FS20}/ms5_{FS20} homozygous male sterile plants.

TABLE 5
Seed set in T0 wheat plants containing a TaMs5
complementation T-DNA insertion.

| T-DNA | | T-DNA | Male |
|---|---|---|---|
| Insertion | ms5_{FS20} | copy | Fertility |
| Event | genotype | number | Phenotpye |
| Event-1 | ms5_{FS20}/ms5_{FS20} | 1 | Fertile |
| Event-2 | ms5_{FS20}/ms5_{FS20} | 2 | Fertile |
| Event-3 | ms5_{FS20}/ms5_{FS20} | 7 | Fertile |
| Event-4 | ms5_{FS20}/ms5_{FS20} | 8 | Fertile |
| Event-5 | ms5_{FS20}/ms5_{FS20} | 2 | Fertile |
| Event-6 | Ms5_{FS20}/ms5_{FS20} | 1 | Fertile |
| Event-7 | Ms5_{FS20}/ms5_{FS20} | 2 | Fertile |
| Event-8 | ms5_{FS20}/ms5_{FS20} | 1 | Fertile |
| No T-DNA | ms5_{FS20}/ms5_{FS20} | 0 | sterile |
| No T-DNA | ms5_{FS20}/ms5_{FS20} | 0 | sterile |

### T1 Analysis; Molecular and Phenotypic

Inheritance of complementation by TaMs5 T-DNA insertion was shown by analyzing the T1 plants derived from 2 separate T0 plants with independent T-DNA insertions (Event-1 and Event-8). One set of T1 progeny was derived from a TO plant homozygous for ms5_{FS20} mutation (ms5_{FS20}/ms5_{FS20}) with TaMs5-UbiBar cassette (Event-1). The second set of T1 progeny was derived from a T0 plant heterozygous for ms5_{FS20} mutation (Ms5_{FS20}/ms5_{FS20}) with TaMs5-UbiBar cassette (Event-8). Plants from both sets were genotyped for ms5 and the T-DNA insertion (Event-1 or Event-8). In both sets of T1 progeny, all the plants with genotype ms5_{FS20}/ms5_{FS20} and T-DNA insertion (Event-1 or Event-8) were fertile as determined by production of seed (Table 6). All the progeny with genotype ms5_{FS20}/ms5_{FS20} without the T-DNA insertion were male sterile and did not produce seed. This clearly demonstrates that the TaMs5 complementation T-DNA insertion is able to restore fertility to the ms5_{FS20}/ms5_{FS20} mutant plants and this ability is passed on to progeny.

TABLE 6
Fertility of T1 plants with or without a TsMs5
complementation T-DNA insertion.

| | | | T-DNA | Male |
|---|---|---|---|---|
| T0 | T1 | ms5_{FS20} | Copy | Fertility |
| Event | Plant | genotype | number | Phenotype |
| Event-1 | Plant 1 | homozygous | 2 | Fertile |
| Event-1 | Plant 2 | homozygous | 0 | Sterile |
| Event-1 | Plant 3 | homozygous | 2 | Fertile |
| Event-1 | Plant 4 | homozygous | 0 | Sterile |
| Event-1 | Plant 5 | homozygous | 0 | Sterile |
| Event-1 | Plant 6 | homozygous | 1 | Fertile |
| Event-1 | Plant 7 | homozygous | 2 | Fertile |
| Event-1 | Plant 8 | homozygous | 2 | Fertile |
| Event-1 | Plant 9 | homozygous | 1 | Fertile |
| Event-1 | Plant 10 | homozygous | 0 | Sterile |
| Event-1 | Plant 11 | homozygous | 2 | Fertile |
| Event-1 | Plant 12 | homozygous | 1 | Fertile |
| Event-1 | Plant 13 | homozygous | 1 | Fertile |
| Event-1 | Plant 14 | homozygous | 1 | Fertile |
| Event-1 | Plant 15 | homozygous | 1 | Fertile |
| Event-1 | Plant 16 | homozygous | 0 | Sterile |
| Event-1 | Plant 17 | homozygous | 2 | Fertile |
| Event-1 | Plant 18 | homozygous | 1 | Fertile |
| Event-1 | Plant 19 | homozygous | 1 | Fertile |
| Event-1 | Plant 20 | homozygous | 1 | Fertile |
| Event-1 | Plant 21 | homozygous | 1 | Fertile |
| Event-1 | Plant 22 | homozygous | 0 | Sterile |
| Event-1 | Plant 23 | homozygous | 1 | Fertile |
| Event-1 | Plant 24 | homozygous | 1 | Fertile |
| Event-1 | Plant 25 | homozygous | 1 | Fertile |
| Event-8 | Plant 1 | homozygous | 1 | Fertile |
| Event-8 | Plant 2 | homozygous | 2 | Fertile |
| Event-8 | Plant 3 | homozygous | 0 | Sterile |
| Event-8 | Plant 4 | homozygous | 0 | Sterile |
| Event-8 | Plant 5 | homozygous | 1 | Fertile |
| Event-8 | Plant 6 | homozygous | 1 | Fertile |

In conclusion, analysis of the T0 and T1 plants with the T-DNA insertion containing the native wheat MS5 gene showed that this gene is able to restore fertility to the ms5_{FS20}/ms5_{FS20} homozygous recessive mutation. This example is a further proof that the ms5_{FS20} mutation is in the wheat Ms5 gene.

### Example 12. Inbred Maintenance and Increase of Wheat Ms5 Male-Sterile Plants Using a Hemizygous Maintainer

This example demonstrates that wheat plants homozygous recessive for ms5 and which are additionally homozygous for a non-functional TaLTPG2-3D allele can be maintained as male-sterile plants using a functional copy of Ms5 linked to a seed marker gene and pollen inhibition gene.

It would be advantageous to produce a pure line of male-sterile plants to allow for cross pollination with a different inbred wheat variety to produce hybrid seed. Generally, strategies that incorporate recessive male sterility result in plants that cannot self-pollinate. To accomplish self-pollination and the production of a pure line of male-sterile plants for cross pollination, an expression cassette (Ms5-AA-Red) is constructed which comprises a functional copy of Ms5 linked to the maize PG47 promoter expressing a functional alpha amylase gene (see, for example, SEQ ID NO:26 in U.S. Pat. No. 8,614,367) and further linked to a color-marker gene (for example, encoding a red fluorescent protein) under control of the barley LTP2 promoter (see, e.g., U.S. Pat. No. 5,525,716) and also carrying a PINII terminator sequence. Using biolistic or Agrobacterium-mediated transformation, this construct is transformed directly into embryos derived from self-pollinated Ms5/ms5 wheat plants which are homozygous for a non-functional TaLTPG2-3D allele. Transformed embryos are regenerated into plants. Wheat plants (ms5/ms5) containing single-copy Ms5-AA-Red cassette, which can be identified using markers flanking the ms5 locus as described above, are male-fertile and are allowed to self-pollinate. Due to the action of PG47: AA to inhibit pollen function and thus prevent transmission of the Ms5-AA-Red expression cassette through pollen, seed from this generation of progeny will segregate at a frequency of 1:1 red-fluorescence and non-fluorescence. Progeny grown from red-fluorescing seed are hemizygous for Ms5-AA-Red, homozygous for ms5, and male fertile; these are used to propagate (i.e., “maintain”) the male-sterile inbred. Progeny of the non-fluorescing seed do not contain a transformed copy of the Ms5 complementing gene, are homozygous for ms5 and male-sterile. These male-sterile inbreds are used as the female inbred for the production of hybrid seed when planted adjacent to male inbred wheat plants that are wild-type for the Ms5 gene.

### Example 13: Targeted Regulation or Mutagenesis of Gene Candidate

For male fertility applications, it may be advantageous to mutate the endogenous Ms1 or Ms5 gene or change its expression, such as by methods described in this example.

Introducing an RNA into a living cell has been shown to inhibit expression of a target gene in that cell (Fire et al. 1998; Timmons and Fire 1998; Fire et al. 1999; Mette et al. 2000; Yu et al. 2002; Cigan et al. 2005; Dalakouras et al. 2009; Bae et al. 2010; Cigan et al. 2010; Tang 2013). A skilled artisan will appreciate that the RNA could be expressed within the cell or applied exogenously (Tang 2013). Interfering RNA may target transcription, translation or mRNA stability, thereby changing the expression of the targeted gene. In this example, expression of the Ms5 gene is reduced or silenced by expressing in planta either RNAs that target the promoter region, as has been shown previously in monocots (Cigan et al. 2010) including wheat (U.S. patent application Ser. No. 14/203,698), or RNAs that target the expressed mRNA, either individually or in combination. For the promoter inverted repeat approach, a portion of the Ms5 promoter region may be duplicated, juxtaposed and oriented in tandem in opposite directions and placed under the control of a constitutive, tissue-preferred or conditional promoter in a plant transformation vector, for the purpose of expressing the promoter inverted repeat RNA in plant cells to silence a gene operably linked to the target promoter.

The skilled artisan will further appreciate that changes can be introduced by mutation of the nucleic acid sequences, thereby leading to changes in either the expression of encoded mRNAs or the amino acid sequence of the encoded Ms5 polypeptide, resulting in alteration of the biological activity of the mRNA or protein, respectively, or both. See for example methods described in U.S. patent application Ser. No. 14/463,687 filed on Aug. 20, 2014, incorporated by reference in its entirety herein. Thus, variant nucleic acid molecules can be created by introducing one or more nucleotide substitutions, additions and/or deletions into the corresponding nucleic acid sequence or surrounding sequences disclosed herein. Such variant nucleic acid sequences are also encompassed by the present disclosure.

Variant nucleic acid sequences can be made by introducing sequence changes randomly along all or part of the Ms5 genic region, including, but not limited to, chemical or irradiation mutagenesis and oligonucleotide-mediated mutagenesis (OMM) (Beetham et al. 1999; Okuzaki and Toriyama 2004). Alternatively, or additionally, sequence changes can be introduced at specific selected sites using double-strand-break technologies such as ZNFs, custom designed homing endonucleases, TALENs, CRISPR/CAS (also referred to as guide RNA/Cas endonuclease systems (U.S. patent application Ser. No. 14/463,687 filed on Aug. 20, 2014)), or other protein and/or nucleic acid based mutagenesis technologies. The resultant variants can be screened for altered Ms5 activity. It will be appreciated that the techniques are often not mutually exclusive. Indeed, the various methods can be used singly or in combination, in parallel or in series, to create or access diverse sequence variants.

### Example 14: Cytological and Metabolite Analysis of Ms5 and ms5_{FS20} Pollen

### Electron and Light Microscopy

Sterile (ms5) and fertile (Ms5) mature anthers before dehiscence were fixed with either paraformaldehyde 4%, glutaraldehyde 1.25%, and sucrose 4% in phosphate-buffered saline (PBS) pH 7.4, for 16 h at 4° C. for scanning electron microscopy (SEM) or 3% glutaraldehyde in 0.1 M phosphate buffer pH 7.0 overnight for transmission electron (TEM) or light microscopy. Samples for SEM were rinsed twice with PBS pH 7.4 for 5 min whereas samples for TEM and light microscopy were washed twice with 1×PBS and embedded in 2% low melting point agarose (Sigma, St. Louis, MO) in 1×PBS for sample orientation and sectioning, then dehydrated using a series of graded ethanol solutions (30%, 50%, 70%, 85%, 90% and 95%) each for 60 min. Samples were then infiltrated 3 times, each for 60 min, in 100% ethanol. Samples were either embedded in LR white resin, sectioned (2 μm) and stained with 0.05% toluidine blue stain and mounted on slides in DPX solution (Sigma, St. Louis, MO) for light microscopy or dissected then critical point dried and sputter coated with platinum (BalTec CPD030 Critical Point Dryer) for SEM. 70-80 nm ultrathin anther sections were prepared and stained in 4% uranyl acetate followed by Reynold's lead citrate (The University of Adelaide microscopy) 43. SEM and image capture was performed at an accelerating voltage of 10 kV (Philips XL20 SEM w EDAX EDS) whereas TEM and image capture was performed on a Phillips CM-1000 TEM (The University of Adelaide microscopy). Light microscopy images were captured using a Zeiss Axio Imager M2 optical microscope (Zeiss, Germany).

### Fatty Acid Profiling

Approximately 50 frozen anthers were transferred into pre-chilled cryogenic mill tubes and weighed accurately. A 300 μL aliquot of 1:3:1 chloroform:methanol:water containing 30 UM internal standard (13C1 Myristic acid) was added to each sample tube. Dried samples and a fatty acid calibration mix (Supelco® 37 Component FAME Mix) was prepared by adding 25 μL of 2:1 chloroform:methanol followed by shaking at 37° C. for 30 minutes. Samples were then derivatised using 5 μL of Meth-Prep II (Grace Davison Discovery). 1 μL was injected onto the GC column. The GC-MS apparatus comprised of a Gerstel 2.5.2 Autosampler, a 7890A Agilent gas chromatograph and a 5975C Agilent quadrupole mass spectrometer (Agilent, Santa Clara, USA). The mass spectrometer was calibrated according to manufacturer's recommendations using tris-(perfluorobutyl)-amine (CF43).

Gas chromatography was performed on a VF-5 MS column (Agilent Technologies, Australia). The injection temperature was set at 250° C., with the MS transfer line at 280° C., the ion source adjusted to 250° C. and the quadrupole at 150° C. Helium was used as the carrier gas at a flow rate of 1.1 mL min-1. The corresponding GC-MS method was performed using the following temperature program; start at injection 50° C., hold for 1 min, followed by a 15° C. min-1 oven temperature ramp to 230° C.; hold for 3 min, followed by a 10° C. ramp to 300° C.

Mass spectra were recorded at 2 scan s-1 with an m/z 50-600 scanning range. Both chromatograms and mass spectra were evaluated using the MassHunter Workstation software version B.07.00 (Agilent, Santa Clara, USA). Retention times and mass spectra (unique qualifier ions) were identified and compared directly to standards from a commercially available fatty acid methyl ester mix (Supelco®37 Component FAME Mix, 47885-U, Sigma-Aldrich). All fatty acid methyl esters identified were quantified using prepared calibration curves from the stock Supelco® 37 Component FAME Mix in the linear range from 2.5-150® M for each lipid class.

Analysis of ms5 anthers revealed disrupted pollen exine structure, which was first observed in early uninucleate microspores and typified by shallow and incomplete exine surface and reduced electron dense materials at the tapetal cell surface. Furthermore, metabolomic profiling by GC-MS revealed that ms5 anthers accumulate lipid monomers of sporopollenin relative to wild-type. Sterile ms5 anthers containing uninucleate microspores exhibited a five fold increase in C16:0 long chain fatty acids whereas C18ln9c, C18:2n6c and C18:3n6 long chain fatty acids increased 14, 23 and 14 fold respectively (Tables 7 and 8). Taken together this suggests Ms5 is necessary for sporopollenin biosynthesis or transport. Transcriptional profiling of wild-type Ms5 by qRT-PCR using the primers in SEQ SEQ ID NOs: 132-137 revealed the A-genome to be preferentially expressed during early microspore development.

TABLE 7
Fatty Acid profiling of Ms5 fertile anthers

| | Ms5 Fertile | | | Ms5 Fertile | | | Ms5 Fertile Anthers | | |
|---|---|---|---|---|---|---|---|---|---|
| | Premeiotic | | | Meiotic | | | with Uninucleate | | |
| Fatty | anthers | | | anthers | | | Microspores | | |
| Acid | x-fold | | sem | x-fold | | sem | x-fold | | sem |
| C10:0 | 1.000 | ± | 0.189 | 0.980 | ± | 0.088 | 1.479 | ± | 0.121 |
| C11:0 | 1.000 | ± | 0.251 | 0.610 | ± | 0.098 | 0.872 | ± | 0.146 |
| C12:0 | 1.000 | ± | 0.183 | 0.992 | ± | 0.070 | 1.509 | ± | 0.108 |
| C14:0 | 1.000 | ± | 0.204 | 0.956 | ± | 0.047 | 1.402 | ± | 0.100 |
| C15:0 | 1.000 | ± | 0.193 | 1.006 | ± | 0.056 | 1.533 | ± | 0.119 |
| C15:1 | 1.000 | ± | 0.179 | 1.009 | ± | 0.087 | 1.553 | ± | 0.117 |
| C16:0 | 1.000 | ± | 0.311 | 1.066 | ± | 0.196 | 1.454 | ± | 0.060 |
| C16:1 | 1.000 | ± | 0.214 | 1.065 | ± | 0.060 | 1.801 | ± | 0.068* |
| C17:0 | 1.000 | ± | 0.190 | 1.026 | ± | 0.063 | 1.551 | ± | 0.094 |
| C17:1 | 1.000 | ± | 0.185 | 1.066 | ± | 0.059 | 1.546 | ± | 0.086 |
| C18:0 | 1.000 | ± | 0.139 | 0.960 | ± | 0.084 | 1.315 | ± | 0.082 |
| C18:1n9c | 1.000 | ± | 0.309 | 2.097 | ± | 0.274 | 2.629 | ± | 0.167* |
| C18:1n9t | 1.000 | ± | 0.309 | 1.295 | ± | 0.209 | 2.129 | ± | 0.025 |
| C18:2n6c | 1.000 | ± | 0.443 | 1.427 | ± | 0.269 | 2.599 | ± | 0.066 |
| C18:3n3 | 1.000 | ± | 0.140 | 0.895 | ± | 0.092 | 1.354 | ± | 0.120 |
| C18:3n6 | 1.000 | ± | 0.423 | 1.282 | ± | 0.286 | 1.610 | ± | 0.105 |
| C20:0 | 1.000 | ± | 0.186 | 1.905 | ± | 0.196 | 1.991 | ± | 0.070* |
| C20:1 | 1.000 | ± | 0.256 | 1.541 | ± | 0.202 | 2.128 | ± | 0.065* |
| C20:2 | 1.000 | ± | 0.215 | 1.106 | ± | 0.059 | 1.675 | ± | 0.060 |
| C20:4n6 | 1.000 | ± | 0.181 | 0.979 | ± | 0.088 | 1.580 | ± | 0.100 |
| C20:5n3 | 1.000 | ± | 0.177 | 1.015 | ± | 0.076 | 1.593 | ± | 0.098 |
| C21:0 | 1.000 | ± | 0.188 | 1.046 | ± | 0.060 | 1.599 | ± | 0.106 |
| C22 | 1.000 | ± | 0.199 | 1.195 | ± | 0.060 | 1.722 | ± | 0.089 |
| C23:0 | 1.000 | ± | 0.182 | 1.055 | ± | 0.059 | 1.603 | ± | 0.105 |
| C24:0 | 1.000 | ± | 0.184 | 1.082 | ± | 0.060 | 1.651 | ± | 0.094 |
| C24:1 | 1.000 | ± | 0.182 | 1.042 | ± | 0.058 | 1.589 | ± | 0.106 |

TABLE 8
Fatty Acid profiling of ms5 sterile anthers

| ms5 Stertile | | | ms5 Sterile | | | ms5 Sterile Anthers with | | |
|---|---|---|---|---|---|---|---|---|
| Premeiotic anthers | | | Meiotic anthers | | | Uninucleate Microspores | | |
| x-fold | | sem | x-fold | | sem | x-fold | | sem |
| 1.000 | ± | 0.107 | 1.284 | ± | 0.141 | 1.494 | ± | 0.384 |
| 1.000 | ± | 0.030 | 1.272 | ± | 0.131 | 1.676 | ± | 0.440 |
| 1.000 | ± | 0.107 | 1.294 | ± | 0.139 | 1.555 | ± | 0.355 |
| 1.000 | ± | 0.086 | 1.388 | ± | 0.116 | 1.765 | ± | 0.294 |
| 1.000 | ± | 0.103 | 1.322 | ± | 0.126 | 1.663 | ± | 0.332 |
| 1.000 | ± | 0.106 | 1.288 | ± | 0.141 | 1.529 | ± | 0.391 |
| 1.000 | ± | 0.216 | 2.410 | ± | 0.126* | 5.679 | ± | 0.064* |
| 1.000 | ± | 0.078 | 1.489 | ± | 0.133 | 2.542 | ± | 0.077* |
| 1.000 | ± | 0.110 | 1.351 | ± | 0.124 | 1.737 | ± | 0.302 |
| 1.000 | ± | 0.101 | 1.333 | ± | 0.125 | 1.734 | ± | 0.298 |
| 1.000 | ± | 0.071 | 1.389 | ± | 0.132 | 2.482 | ± | 0.127* |
| 1.000 | ± | 0.493 | 5.164 | ± | 0.174* | 13.639 | ± | 0.219* |
| 1.000 | ± | 0.175 | 1.825 | ± | 0.137 | 5.674 | ± | 0.260* |
| 1.000 | ± | 0.432 | 4.896 | ± | 0.247 | 22.796 | ± | 0.341* |
| 1.000 | ± | 0.250 | 0.842 | ± | 0.124 | 0.965 | ± | 0.386 |
| 1.000 | ± | 0.558 | 4.372 | ± | 0.214 | 14.104 | ± | 0.229* |
| 1.000 | ± | 0.013 | 2.333 | ± | 0.089* | 3.833 | ± | 0.122* |
| 1.000 | ± | 0.017 | 2.097 | ± | 0.080* | 4.381 | ± | 0.127* |
| 1.000 | ± | 0.283 | 1.103 | ± | 0.114 | 1.685 | ± | 0.157 |
| 1.000 | ± | 0.109 | 1.284 | ± | 0.143 | 1.550 | ± | 0.367 |
| 1.000 | ± | 0.108 | 1.306 | ± | 0.144 | 1.534 | ± | 0.373 |
| 1.000 | ± | 0.104 | 1.339 | ± | 0.131 | 1.637 | ± | 0.339 |
| 1.000 | ± | 0.096 | 1.456 | ± | 0.102 | 2.067 | ± | 0.226 |
| 1.000 | ± | 0.182 | 1.218 | ± | 0.130 | 1.545 | ± | 0.314 |
| 1.000 | ± | 0.106 | 1.351 | ± | 0.128 | 1.760 | ± | 0.291 |
| 1.000 | ± | 0.144 | 1.265 | ± | 0.136 | 1.600 | ± | 0.311 |

Tables 7 and 8: Fatty Acid profiling of Ms5 fertile versus ms5 sterile anthers. Mean fatty acid content and associated standard error (SEM) was calculated as concentration in mmol per anther based on three biological replicates and presented as a fold change relative to pre-meiotic anthers for each fertile and sterile anther sample. * indicates those samples that have a T-test value below p<0.05, but not below the Bonferroni corrected p-value.

1-31. (canceled)

32. A method for producing a male-sterile wheat plant, the method comprising:
a) introducing a genetic modification into at least one or more endogenous MS1 polynucleotide sequences in a wheat plant cell, wherein the genetic modification confers male sterility to a wheat plant from the wheat plant cell, wherein the at least one or more endogenous MS1 polynucleotide sequences is selected from the group consisting of:
(1) a polynucleotide comprising the sequence set forth in SEQ ID NO:1-3, 5-8, 10-13, or 15;
(2) a polynucleotide having at least 85%, 90% or 95% sequence identity to SEQ ID NO: 1-3, 5-8, 10-13, or 15;
(3) a polynucleotide that encodes a polypeptide having at least 85%, 90% or 95% sequence identity to SEQ ID NO:4, 9; or 14
(4) a polynucleotide that encodes a polypeptide of SEQ ID NO: 4, 9; or 14; and
b) obtaining the male-sterile wheat plant from the wheat plant cell.

33. The method of claim 32, wherein said the genetic modification is introduced by a TALEN, a meganuclease, a zinc finger nuclease, or a CRISPR-associated nuclease.

34. The method of claim 32, wherein the genetic modification introduces one or more nucleotide substitutions, additions and/or deletions into at least one or more endogenous wheat MS1 polynucleotide sequences.

35. The method of claim 32 further comprising selecting the male sterile wheat plant, wherein the wheat plant comprises the genetic modification in the at least one or more endogenous wheat MS1 polynucleotide sequences.

36. The method of claim 32, further comprising crossing the male-sterile wheat plant with a male-fertile wheat plant to produce a hybrid wheat seed.

37. A wheat plant having modified male-fertility comprising at least one altered target site that confers modified male-fertility to the wheat plant, wherein the at least one altered target site originated from an endonuclease, and wherein the at least one altered target site is located in or near an endogenous wheat male fertility gene of MS1, wherein the endogenous wheat male fertility gene of Ms1 comprises a wheat Ms1 polynucleotide sequence selected from the group consisting of: (a) a polynucleotide comprising the sequence set forth in SEQ ID NO: 1-3, 5-8, 10-13, or 15; (b) a polynucleotide having at least 85%, 90% or 95% sequence identity to SEQ ID NO: 11-3, 5-8, 10-13, or 15; (c) a polynucleotide that encodes a polypeptide having at least 85%, 90% or 95% sequence identity to SEQ ID NO: 4, 9; or 14; (d) a polynucleotide that encodes a polypeptide of SEQ ID NO: 4, 9; or 14, and the altered target site affects the expression level of the MS1 gene so that the wheat plant is male-sterile.

38. A method for restoring male fertility, the method comprising:
a) introducing a genetic modification into at least one or more endogenous wheat MS1 polynucleotide sequences in a wheat plant cell, wherein the genetic modification confers male sterility to a wheat plant obtained from the plant cell, wherein the genetic modification is introduced by an endonuclease guided by at least one guide RNA; and
b) introducing into and expressing in the wheat plant cell one or more polynucleotides that encode a MS1 polypeptide that confers male fertility to the male-sterile wheat plant obtained from the wheat plant cell.

39. The method of claim 38, wherein the genetic modification introduces one or more nucleotide substitutions, additions and/or deletions into at least one or more endogenous wheat MS1 polynucleotide sequences.

40. The method of claim 38, wherein the at least one or more endogenous wheat MS1 polynucleotide sequences is selected from the group consisting of:
(a) a polynucleotide comprising the sequence set forth in SEQ ID NO: 1-3, 5-8, 10-13, or 15;
(b) a polynucleotide having at least 85%, 90% or 95% sequence identity to SEQ ID NO: 1-3, 5-8, 10-13, or 15;
(c) a polynucleotide that encodes a polypeptide having at least 85%, 90% or 95% sequence identity to SEQ ID NO: 1-3, 5-8, 10-13, or 15; and
(d) a polynucleotide that encodes a polypeptide of SEQ ID NO: 4, 9; or 14.

41. The method of claim 38, wherein said the genetic modification is introduced by a TALEN, a meganuclease, a zinc finger nuclease, or a CRISPR-associated nuclease.

42. The method of claim 38, wherein the genetic modification is introduced by a Cas9 endonuclease guided by at least one guide RNA.

43. The method of claim 38, wherein the MS1 polypeptide that confers male fertility to the male-sterile wheat plant is from a monocot.

44. The method of claim 38, wherein the MS1 polypeptide that confers male fertility to the male-sterile wheat plant is from wheat or a wheat species.

45. The method of claim 38, wherein the MS1 polypeptide that confers male fertility to the male-sterile wheat plant is from a species different than wheat.

46. The method of claim 38, the method further comprising obtaining a MS1 male-fertile wheat plant from the wheat plant cell.

47. The method of claim 38, further comprising crossing the male-fertile wheat plant with itself to produce wheat seed.

48. A wheat plant having restored male-fertility, the wheat plant comprising at least one altered target site that confers modified male-fertility to the wheat plant, wherein the at least one altered target site originated from a corresponding target site that was recognized and cleaved by an endonuclease, and wherein the at least one altered target site is located in or near one or more endogenous wheat male fertility polynucleotides of MS1 and the altered target site affects the expression level of the one or more endogenous MS1 polynucleotides so that the wheat plant is male-sterile and wherein the wheat plant comprises one or more polynucleotides that encodes a MS1 polypeptide that confers male fertility to the male-sterile wheat plant when expressed in the wheat plant's male-tissue.


SEQUENCE LISTING (212 sequences)

SEQ ID NO: 1 - DNA, 1857 bp, Triticum aestivum
  source 1..1857: mol_type = genomic DNA
atggagagatcccgccgcctgctgctggtggcgggcctgctcgccgcgctgctcccggcggcggcggccgccttcgggcagcagccgggggcgccgtgcgagcccacgctgctggcgacgcaggtggcgctcttctgcgcgcccgacatgcccaccgcgcagtgctgcgagcccgtcgtcgccgccgtcgacctcggcggcggggtcccctgcctctgccgcgtcgccgcggagccgcagctcgtcatggcgggcctcaacgccacccacctcctcacgctctacagctcctgcggcggcctccgtcccggcggcgcccacctcgccgccgcctgcgaaggtacgcgcacgttcaccgccctccgtccctccctctctctgtctacgtgcagattttctgtgctctctttcctgcttgcctagtacgtagtgttccatggcctctcgggccgctagcgctccgatttgcgttggtttccttgctgttctgccggatctgttggcacggcgcgcggcgtcgggttctcgccgtctcccgtggcgagcgacctgcgcagcgcgcgcggcctggctagcttcataccgctgtaccttgagatacacggagcgatttagggtctactctgagtatttcgtcatcgtaggatgcatgtgccgctcgcgattgtttcatcgatttgagatctgtgcttgttcccgcgagttaagatggatctagcgccgtacgcagatgcagagtctgttgctcgagttaccttatctaccgtcgttcgactatggtatttgcctgcttccttttggctgggtttatcgtgcagtagtagtagacatgtggacgcgttcttcttattttgtgccgaccatcgtcgagatacttttcctgctacagcgtttcatcgcctgcaccatcccgttcgtgatagcacttttgtgtcaaaccgcaacgcagctttgctttctgcggtatcttctgccttgtttgtcgccttgcttggtcaaaactgagaactcttgctgtttgatcgaccgagggcagaggcagagcaagagcctgccgtgcttttggctctgcagtgcgtcgtctctgcctcctttgccaaacatttccatgttgatcctctgggggcactgctttttcgcatgcggtttccgtagccttcctctttcatgaaaaaaggtttgggtcaaatcaaatggatcgcctattggcagagcagcagcagatagctggctgtctcacagctttggcagaatcggtctgttgcctgccaccgtgtctcttatcttgcctgccaccgtgtctcttttcttgttgcgcacgtcgtcacctcctcctacttcttttccagttttgtttacttttgatgaaatacggacgaacggctggtaatcattaactttggttgctgttgttactgtggattttggacgcaggacccgctcccccggccgccgtcgtcagcagccccccgcccccgccaccgtcgaccgcacctcgccgcaagcagccagcgcgtgcgtacctctccctctcgcccgcatctcgctccgtattaactgattgtgtctgcatactgacgtgtgctttggctttggatctgtttcgcagacgacgcaccaccgccgccgccgccgtccagcgacaagccgtcgtccccgccgccgtcccaggaacacgacggcgccgctccccacgccaaggccgcccccgcccaggcggctacctccccgctcgcgcccgctgctgccatcgccccgccgccccaggcgccacactccgccgcgcccacggcgtcatccaaggcggccttcttcttcgtcgccacggccatgctcggcctctacatcatcctctga

SEQ ID NO: 2 - DNA, 1997 bp, Triticum aestivum
  source 1..1997: mol_type = genomic DNA
ttctgcaaaaatctccactagccattgcataagctcaggaaaattacctttatgtagtgaaacttctctccatcatgttcacgaaaatctaacccttgacaaaaaaggaacctcgggcattaaaaggaatatgtcaggccagctctatataaaaccttgtctcgtttgatggttgaacaaaatgactctatgattgttgtgtttgctgcaatgaagaaattgtatttctcttgtgctttgttacgtgcacactgcactattgatttcaccgacatgtttcacaaaactatccttgtgattctaatttctaagtcacccattcaccaaaaatctccaccaacatgcaaattatcattgaaaagataacatacaagcataaagcaccatctagttctttactatactcaagccaactataagacttaaaccatttagctacaaatattgttgcacacctccggtggggtgttgtggaaaagcatattttttcggtcaacaagccccttttgcaatgtatcctcttctaatcctattcggaccattaacatcataagttgcgattggcatcctcttcctaggatcagattcactcaatcgaacatcataaactgcatcttcaatgtcacccatttcctatattttttcagattattggcttgcttcgttcgcaatattaggtactgtgattggacttctgttgatgccactaataatttgcagttgttgcggaatatgaactcaaggggagctcatggtgctatgaagttgattcggtgggaaattgttctacatccgcacttgctgctcaacctaaatacatgggttggatttcttcccaactttagtacataaagttctcaaattaatgttctactacattaaaattgaaatccgcaaacattttttagtacccaaacatttttctaatatacggtgaacatttttcatctactgattttttgatatatggtgaaaattggtgtaatatatgctggcatgtttttaaatactacatattgaccatgtagataaaaaatttatagtatatgatgaacatttttgtaatatagatggccatttttaaaatatacattgcacattttataatatacgatgagcagtttataatactagatgaaccttttttggagttctgaacatttttttgaaaacagcagccattgtacaagaaaaaaccaaaacaaaagaaatgagaaacccaaaaacaaaaacaaaacaaaacaaaacagagaaacctacagaaaaaaacgaaacagaaaaaggcaaaggaagaacccgaactgggccagccggctcggcgtgccccagtgggccgtcgtggcgaatgcaacggctacatgggccgctcttcgtgaaagagaaggaggtcagttcatggaccgctaccagtacacgggcctcgctgtggcaacacccgccgtgtactagttttcgcgggaatccaatgccaaaatcgctccccgcgggaacccgacgtcggtctggtgacttctggagccttccagaacactccacaagctcccagagccgtctgatcagatcagcacgaagcacgaacattggcgcgcgaagatattttccttcccgacgacgccacactgcatttcatttgaatttcaaaaatcgaaaacggaaaacactttctctcatcccgaggagaggcggttagtgccagaggagcacgagagaggccaccccccccccagccagctcacgtgccgtgccctcgcaccctgcgcggccgcatccgggccgtccgcgcggacagctggccgcgccccacccgaaccgacgcccaggatcgcgcccgccacccgcttgccttagcgtccacggctcctccggctatataacccgcccctcacccgctccccctccggcattccatttccgtcccaccaccgcaccaccaccactccaccaaaaccctagcgggcgagcgagggagagagagaccaccccgcccgaccccgccg

SEQ ID NO: 3 - DNA, 669 bp, Triticum aestivum
  source 1..669: mol_type = genomic DNA
atggagagatcccgccgcctgctgctggtggcgggcctgctcgccgcgctgctcccggcggcggcggccgccttcgggcagcagccgggggcgccgtgcgagcccacgctgctggcgacgcaggtggcgctcttctgcgcgcccgacatgcccaccgcgcagtgctgcgagcccgtcgtcgccgccgtcgacctcggcggcggggtcccctgcctctgccgcgtcgccgcggagccgcagctcgtcatggcgggcctcaacgccacccacctcctcacgctctacagctcctgcggcggcctccgtcccggcggcgcccacctcgccgccgcctgcgaaggacccgctcccccggccgccgtcgtcagcagccccccgcccccgccaccgtcgaccgcacctcgccgcaagcagccagcgcacgacgcaccaccgccgccgccgccgtccagcgacaagccgtcgtccccgccgccgtcccaggaacacgacggcgccgctccccacgccaaggccgcccccgcccaggcggctacctccccgctcgcgcccgctgctgccatcgccccgccgccccaggcgccacactccgccgcgcccacggcgtcatccaaggcggccttcttcttcgtcgccacggccatgctcggcctctacatcatcctctga

SEQ ID NO: 4 - protein, 222 aa, Triticum aestivum
MERSRRLLLVAGLLAALLPAAAAAFGQQPGAPCEPTLLATQVALFCAPDMPTAQCCEPVVAAVDLGGGVPCLCRVAAEPQLVMAGLNATHLLTLYSSCGGLRPGGAHLAAACEGPAPPAAVVSSPPPPPPSTAPRRKQPAHDAPPPPPPSSDKPSSPPPSQEHDGAAPHAKAAPAQAATSPLAPAAAIAPPPQAPHSAAPTASSKAAFFFVATAMLGLYIIL

SEQ ID NO: 5 - DNA, 1000 bp, Triticum aestivum
  source 1..1000: mol_type = genomic DNA
gtcgccgaccccgaggccatggtccgtccagttgcagtagaatgctcgtcgtcttgttccgtttcatgcttgtcgccgttcgaggttcgtttctgcagtccgattgagaagaagacggtgggttttgatcgcgtcccgagatttctgttgtcgatcgtagcgtcctggtagtagtagtgtctggtagcagcagtatgttcatgtgtcctcggtcgcctagttttggtctcaagtagtactgtctgtccaccgtgtttgcgtggtcgcggagaacatcattgggttttgcgattcctctggtcagatgaaccactgctatgtgatcgatcgatatgatctgaatggaatggatcaagttttgcgttctgctgatgacgtgatgcttcttcagttatattcatgctcgatctatttctgtttcccccatttgaatttgtggagcagcagtttggctttcttttgttctgctatggatgaatgcttcttgcatgcatcttgtctttgcttaatttgaactgtagaacggatgcagttctggtttctgctaatgatgtgatgattcttcatatgcatatgctttacatgttcatctcttcaaatttgtgcagcaacagtttgtagctttcattcggctctgaatgaaatgcctcttgcatgttgtctttgcttaatttgtttttcacggggagcctgctgcagctttctgttgccatgttgttttccacgccaggacaaaatagatggtgcggtttgattcgatcccggttaattgcttgatgctagcttctgatcaatcccttcatcacgatgttccggagagccacatggaactggaggggggagattcaaattcatgcatgcaaatttgtgttggtgttgggtcacgtcaagcagtcactttttgcagtatcactcttaccattttatccttttgttgaaacctctctcctcaccccaaaagttgatgcaatagtgctatgcccacccatgcttttttcat

SEQ ID NO: 6 - DNA, 1856 bp, Triticum aestivum
  source 1..1856: mol_type = genomic DNA
atggagagatcccgcgggctgctgctggtggcggggctgctggcggcgctgctgccggcggcggcggcgcagccgggggcgccgtgcgagcccgcgctgctggcgacgcaggtggcgctcttctgcgcgcccgacatgccgacggcccagtgctgcgagcccgtcgtcgccgccgtcgacctcggcggcggggtgccctgcctctgccgcgtcgccgccgagccgcagctcgtcatggcgggcctcaacgccacccacctcctcacgctctacagctcctgcggcggcctccgccccggcggcgcccacctcgccgccgcctgcgaaggtacgttgtccgcctcctcccctccctccctccctccctctctctctacgtgctcgctttcctgcttacctagtagtacgtagtttcccatgccttcttgactcgctagaagtgctccggtttgggtctgttaatttcctcgctgtactaccggatctgtcgtcggcacggcgcgcggcgtcgggtcctcgccttctcccgtggcgaccgacctgcgcagcgcgcgcgcggcctagctagcttcataccgctgtacctcgacatacacggagcgatctatggtctactctgagtatttcctcatcgtagaacgcatgcgccgctcgcgattgtttcgtcgattctagatccgtgcttgttcccgcgagttagtatgcatctgcgtgcatatgccgtacgcacgcagatgcagagtctgttgctcgagttatctactgtcgttcgctcgaccatatttgcctgttaatttcctgttcatcgtgcatgcagtagtagtagccatgtccacgccttcttgttttgaggcgatcatcgtcgagatccatggctttgctttctgcactatcttctgccttgttttgttctccgcagtacgtacgtcttgcttggtcaaaactgaaaaacgctttgctgtttgtttgatcggcaagagctggccgtgcttttggcaccgcagtgcgtcgcctctgccgcttttgcgaaacatttccatgttgatcctctggcggaactactttttcgcgtgcggtttgcgtggccttcctctctcgtgaaaagaggtcgggtcaaaccaaatggatcgcctcttggcagagcagcggcagcagatagctggccgtctcgcagctttggcagaaccggtctgtggccatctgtcgccgcctgccaccgtttccctgatgtttgtttctctctcgcctgccactgtttcttttcttgttgcgcacgtacgtcgtcacctcctcctacttttttgccagttttgtttacttttgatgaaatatacggatgaatcggctggtgattaactttggctgctgctgttaattactgtggattttggatgcaggacccgctcccccggccgccgtcgtcagcagccccccgcccccgcctccaccgtccgccgcacctcgccgcaagcagccagcgcgtaagaacctctccctctccctctctctctccctctcgcctgcatctcgctatgtttatccatgtccatatgttgatcagccttgtttagttactaacatgtgcaccggatcgggttctcgcagacgacgcaccaccgccgccaccgccgtcgagcgagaagccgtcgtccccgccgccgtcccaggaccacgacggcgccgccccccgcgccaaggccgcgcccgcccaggcggccacctccacgctcgcgcccgccgccgccgccaccgccccgccgccccaggcgccgcactccgccgcgcccacggcgccgtccaaggcggccttcttcttcgtcgccacggccatgctcggcctctacatcatcctctga

SEQ ID NO: 7 - DNA, 2000 bp, Triticum aestivum
  source 1..2000: mol_type = genomic DNA
gcatgaacttattatataataacttgaccgaaaaactaccaaagctggcatgaggccaaataacagagtccattgtctccgagagcaccgggaaggaagcagcaagacagtgccaatcttccaactctacaggggacaacacctatggaaacccaaatcccaattcctatcagagagctccacgatggagatcactatatccaaacaataagaaaacaaggttgggaaagagcccgagagaggcccctctccaacccaccaatcaaaccgaaagcgaacagaacgaccatctcccaccacaaacttaactagagacttgaactcatcatgaacacgaatcaactggcgccagaaccgggagcccccagatccataggcaaacaatgggttgccggtggggaaatgtttagctctcaggatatcccaccatagagtcctagcactagttctttactatactcaagccaactataagacttaaaccatttagttacaaatatcgatgcacaccttcggtggggcgttgtgaaaaagcatgttttttgggtcgacaagccccttttgcaacgtatcctcttctaatcctattcagatcattaacatcataagctgcaattgacatgctcttctgaggatcaggttcatgcaattaaacatcataaactgcatctttgatgtcatccttttcctatattttttccagattattggcttgcttcgttttcaatatcaggttctatgattcgacttctgttgttgccagtaataatttgtagttgctgcggaatatgaactcaaggagagctgatggtgctatgaagttgatttgatgggaggttgttctacacctgcacttgctgctcgacttaaatacatgccttggatttcttcccagctctagtacataatatttttcaaattaatgttccacgacataaaatttaaatccacaaacatatttttagtacatgaacaattttctaatatagggcaaacatttttcatatacaaaccgatcattttaatatatggtgaaaatcagtgtaatatatgctgaaatgttttcaaatacatattgaacatatttataataaatggtgaacattttttttaataattgatgaccatttttaaaatgcatattgaacattttataatatacactgtacagttttataataatcgacgaacatcttttggagttctgaacatttttttcaaaaacacaagccattttccaggaagaatacaaatgcaaaagaaatgagatatccaaaaagcaaaaaagaaaaacaaaacaaaacagagaaacctacaggaaaatccaaacagaaaaggcaaagaaagaacccgaactgggccaggcaatgtttccaacggcctcgctcttcctgaacaagaaggccagtcagcccatgggctgctcccagtactcgggccccgctgtggcagcacgccatgtaatagttttcgcgggaatccaacgccgaaatcgcccgcagcgggaacccgacgtcggtctggtgcgttctggcgccttccagaactctccacaggctcccgcagccgtccgatcagatcagcacgaagcacgaacattggcgcgcggcgatattttctttcctcgcccgacgacggccgcactgcatttcattttgaatttcaaaattcggaaacggaaaagctttctcgcatcccgaggcgaggcggttacgggcgccagaggggccaccccacccacccacccccgccctcacgtgccccgcgcggccgcatccgggccgtccgcgcggacagctggccgcgcccagcccgaaccgacgcccaggatcgagcgagggcggcgcgcccggggcttggcttagcgtccacgccacctccggctatataagccgccccacacccgctccccctccggcattccattccgccaccgcaccaccaccaccaccaaaccctagcgagcgagcgagggagagagagaccgccccgccgcgacg

SEQ ID NO: 8 - DNA, 663 bp, Triticum aestivum
  source 1..663: mol_type = genomic DNA
atggagagatcccgcgggctgctgctggtggcggggctgctggcggcgctgctgccggcggcggcggcgcagccgggggcgccgtgcgagcccgcgctgctggcgacgcaggtggcgctcttctgcgcgcccgacatgccgacggcccagtgctgcgagcccgtcgtcgccgccgtcgacctcggcggcggggtgccctgcctctgccgcgtcgccgccgagccgcagctcgtcatggcgggcctcaacgccacccacctcctcacgctctacagctcctgcggcggcctccgccccggcggcgcccacctcgccgccgcctgcgaaggacccgctcccccggccgccgtcgtcagcagccccccgcccccgcctccaccgtccgccgcacctcgccgcaagcagccagcgcacgacgcaccaccgccgccaccgccgtcgagcgagaagccgtcgtccccgccgccgtcccaggaccacgacggcgccgccccccgcgccaaggccgcgcccgcccaggcggccacctccacgctcgcgcccgccgccgccgccaccgccccgccgccccaggcgccgcactccgccgcgcccacggcgccgtccaaggcggccttcttcttcgtcgccacggccatgctcggcctctacatcatcctctga

SEQ ID NO: 9 - protein, 220 aa, Triticum aestivum
MERSRGLLLVAGLLAALLPAAAAQPGAPCEPALLATQVALFCAPDMPTAQCCEPVVAAVDLGGGVPCLCRVAAEPQLVMAGLNATHLLTLYSSCGGLRPGGAHLAAACEGPAPPAAVVSSPPPPPPPSAAPRRKQPAHDAPPPPPPSSEKPSSPPPSQDHDGAAPRAKAAPAQAATSTLAPAAAATAPPPQAPHSAAPTAPSKAAFFFVATAMLGLYIIL

SEQ ID NO: 10 - DNA, 1000 bp, Triticum aestivum
  source 1..1000: mol_type = genomic DNA
gtcgcgcgccgaccccgcgagagaccgtggtccgtccagtcgcagtagagtagagcgctcgtcgtctcgttccgtttcgtgcctgtcgccgttcgaggttcgtttctgcgtgcagtccggtcgaagaagccggtgggttttgagtactagtggtagtagtagcagcagctatcgtttctgtccgctcgtacgtgtttgcgtggtcgcggagaacaattaattgggtgtttgcgagtcctctggttaagatgaaccactgatgctatgtgatcgatcgatcggtatgatctgaatggaaatggatcaagttttgcgttctgctgatgatgtgatccatttggatctgtgtggggcaacagtttcgcttgcttttgctctgcgatgaacgaatgcttcttgcatgcatcttgtctttgcttaatttgaactgtagaacggatgcagtactgatttctgcttatgatgtgacgattcgtcgtacgcatatcatctcttcaaatttgtgtagcagctgtttgtagcttccattctgctatggacgaatgcctgtttttcacggagaaccgcgcgcggggaccgatgcggctttgtgttgccatgttgttttccacgccaggacaaaatagatggtgcggttttgatccccaatcccaccatcaccatgttccggagagccacatggaactcacgtcaagcggtcactttttgcagaatcactcttaccattttacccttttgttgaaacctctctcctcatccccaaaagttgatgcaacagtgctatgcgcgcccacccatgctttttcatatgattgtaaaatttggatcgattttatcttttgaaccctaagtccggtttacaatctgtttgcatgtttatgttccttgcggcgaggaccattaaacaagactactattggatatatttcgacaggctttgaaatccgaattctaaaacatggtgagactctatgagacacaagaatgctctttagaacac

SEQ ID NO: 11 - DNA, 1895 bp, Triticum aestivum
  source 1..1895: mol_type = genomic DNA
atggagagatcccgcggcctgctgctggcggcgggcctgctggcggcgctgctgccggcggcggcggccgcgttcgggcagcagccgggggcgccgtgcgagcccacgctgctggcgacgcaggtggcgctcttctgcgcgcccgacatgcccacggcccagtgctgcgagcccgtcgtcgccgccgtcgacctcggcggcggggtgccctgcctctgccgcgtcgccgcggagccgcagctcgtcatggcgggcctcaacgccacccacctcctcacgctctacggctcctgcggcggcctccgtcccggcggcgcccacctcgccgccgcctgcgaaggtacgtcgcgcacgttcaccgcctccctccctccctcgctctctctctctctctctctctctctctctacgtgccgattctctgtgttcgcttccctgcttacctagcacgtagttttccatggcttctcgactcgctggtcctccgatttgggtcggttaatttcctcgctgtactaccggatctgtcggcacggcgcgcggcgtcgggttctcgccgtctcccgtggcgagcgacctgcgcagcgcgcgcgcggcctagctagcttcataccgctgtaccttcagatacacggagcgatttagggtctactctgagtatttcgtcatcgtaggatgcatgtggcagtcgcgattgtttcatcgattttagatctgtgcttgttcccgcgagttaagatggatctagcgccgtacgcagacgcagatggtcttgctgtctctgttgctcgagttatcttatctactgtcgttcgagtatatttgcctgcttccttttgatctgtgtttatcgtgcagtagcagtagccatgtccacgccttcttgtttcgaggcgatcatcgtcgagatagcgctttgtttcaaaccgcaacgcagcctttgctttctgcggtatcttctgccttgtttttgttctgtgcagtacgtcttgcttggtcaaaagtaaaaactcttgctgttcgatcgaccgaggcctgatgcagagcaagagctggccgtgcttttcgctctgcagtgcatcgcctctgcctctttggccaaacatttccatgttgatcctctggtgtggtactacttttttgcatgcggtttgcgtagccttcctctttcgtgaaaaaaggtcgggtcgcctattggcagagcagcagcagcagcaacagatagctggctgtctcgcagctttgacagaaccggtctgtggccatctgtcgccgcctgccaccgtttccctgatgtttgtttctctcgtctcatctcgcctgccactgtttcttttcttgttgcgcacgtcgtcacctcctcctacttttttttccagttttgtttacttttgagatacggacgaacggctggtaattactaactttggttgctgttgttactgtggattttggacgcaggacccgctcccccggccgccatcgtcagcagccccccgcccccgccaccaccgtccgccgcacctcgccgcaagcagccagcgcgtacgaacctctccctccctctctctcgcctgcatctcgctctgtattagctgattgtgtttacttactgacgtgtgctttggctttggatctgtttcgcagacgacgcaccgccgccgccgccgccgtctagcgagaagccgtcgtccccgccgccgtcccaggagcacgacggcgccgccccccgcgccaaggccgcgcccgcccaggcgaccacctccccgctcgcgcccgctgccgccatcgccccgccgccccaggcgccacactccgcggcgcccacggcgtcgtccaaggcggccttcttcttcgtcgccacggccatgctcggcctctacatcatcctctga

SEQ ID NO: 12 - DNA, 2000 bp, Triticum aestivum
  source 1..2000: mol_type = genomic DNA
ccaaacaacagagtccactgtctccaagagcaccgggaaggaagcagcaagacggtgccaatcttccaactctacaggggacaacaccctatggaaacccaaatcccaattcctaccagagagctccgtgatggagatccctggatctgagcaataagaaaacaagtttggaaaagagcccgagagaggcccctctccaacccaccaatccaaccgaaagcaaacaaaacgaccatctcccaccacgaacttaactagagacctgaaatcatcatgaacatgaatcagctggcgcaagaaccgggagcccccagatccagaggcaaacaatgggttgccagtggggaaatatttagctttcaggatatcccaccatagggtcctcgcactagttctttactatactcaagccaactataagacttaaaccatttagctacaaatatcgatgcacacctcccgtggggtgttgcggaaaagcatgtttttttggtcgacaagcccctttcacaatgtatcctcttctaattctattcagatcattaacatcagctgtgattgacatcctcttcccaagatcagattcacgcaattgaacatcataaaccacatcttcaatgtcatcctcttcctatatatttttagatgattagcttgcttcgttctcaatatcaggttctatgaatggacttgagttgatgccactaataatttgtagttgttgcaaaatgtgaactgaaggggagctatgaatgaacttgagttgatttgatgggaaattgttctacacatgcacttgctgctcaacttaaatacgtgccttggatttcttcccaactttagtacataaagttctccaagtaatgttctactacataaaatttgaaatctgcaaacattttttagtacacgaacatttttctatatacagtgaacatttttcatctactgattttattttaatatatggtgaaaattggtgtaatatatgctgacatgtttttaagtacatattgaacatatatataaaatacatgatgaacatttttgttatatatgatgctcattttttcaatacatattgaacattttatattatacgatggacagttttataataatcaatgaacaacttttggagttctgaacatgcttttgaaaacacaagacattttccaataaaaaacaaaacaaaagaaatgagaaacccaaaaacaaaaacaaaacaaaacagagaaacctacagaaaaaacgaaacagagaaggcaaagaaagaaccggaactgggccagccaactcggcgtgccccagtggtccgtcgtggcgaatgtttgcaacggctacatgggccgctcctcgtgaaaaagaagaaggtcagtccatgggctgctaccagtacacgggcctcgctgtggcaaactggcaacacgccatattagttttcgcgggaatccaatgccgaaaaccacccaccgcgggaacccgacgtcggtctggtgacttctggcgccttccagaaccctccacaagctcccagagccgtctgatcagatcagcacgaagcacgaagcacgaacattggcgcgcgaagatattttctttccccagcctccgcctcgcccgacgacgccgcactgcatttcatttgaatttcaaaaatcgaaaacggaaaaactttctcgcatcccgaggagaggcggttacgcgcgccagaggagcacgagagaggccaccccacgcacccagccagctcacgtgccgccctcgcaccccccgcggccgcatccgggccgtccgatcgcacagctggccgcgctccacccgaaccgacgcccaggatcgcgcccgccacccgcttgccttcgcgtccacggctcctccggctatataacccgccccccacccgctccccctccggcattccaccccaacaccgcatcaccaccaccactccaccaaaccctagcgaccgagcgagagagggagagaccgccccgccg

SEQ ID NO: 13 - DNA, 672 bp, Triticum aestivum
  source 1..672: mol_type = genomic DNA
atggagagatcccgcggcctgctgctggcggcgggcctgctggcggcgctgctgccggcggcggcggccgcgttcgggcagcagccgggggcgccgtgcgagcccacgctgctggcgacgcaggtggcgctcttctgcgcgcccgacatgcccacggcccagtgctgcgagcccgtcgtcgccgccgtcgacctcggcggcggggtgccctgcctctgccgcgtcgccgcggagccgcagctcgtcatggcgggcctcaacgccacccacctcctcacgctctacggctcctgcggcggcctccgtcccggcggcgcccacctcgccgccgcctgcgaaggacccgctcccccggccgccatcgtcagcagccccccgcccccgccaccaccgtccgccgcacctcgccgcaagcagccagcgcacgacgcaccgccgccgccgccgccgtctagcgagaagccgtcgtccccgccgccgtcccaggagcacgacggcgccgccccccgcgccaaggccgcgcccgcccaggcgaccacctccccgctcgcgcccgctgccgccatcgccccgccgccccaggcgccacactccgcggcgcccacggcgtcgtccaaggcggccttcttcttcgtcgccacggccatgctcggcctctacatcatcctctga

SEQ ID NO: 14 - protein, 223 aa, Triticum aestivum
MERSRGLLLAAGLLAALLPAAAAAFGQQPGAPCEPTLLATQVALFCAPDMPTAQCCEPVVAAVDLGGGVPCLCRVAAEPQLVMAGLNATHLLTLYGSCGGLRPGGAHLAAACEGPAPPAAIVSSPPPPPPPSAAPRRKQPAHDAPPPPPPSSEKPSSPPPSQEHDGAAPRAKAAPAQATTSPLAPAAAIAPPPQAPHSAAPTASSKAAFFFVATAMLGLYIIL

SEQ ID NO: 15 - DNA, 1000 bp, Triticum aestivum
  source 1..1000: mol_type = genomic DNA
gtggccgaccccgcaagaccatgtccgtccagttgcagtagagtagagtgctcgtcgtcttgttccgtttcatgcttgtcgccgttcgaggttcgtctctgcatgcagtccgatcgaagaagacggtggattttgagtagtagctgtcgttggcaggagtatggagttcatgtgtcctcggtcgcctagttttggtctcaagtagtgtctgtctgtccgccgtgtttgcgtggtcgcggagaagtacaattggtgggtgtttgcgattcctctggttagatgaaccactgctatgtgatcgatcgatatgatctgaatggaatggatcaagttttgcgttccgctgatgatgatgtgatatgcttcttcatgtatatatattcatgctcgatctatttgtgtttctccgatttgaatctgtgttaagcaacagtttgtcttgcttctgttctgcagcttctgctatggatggatgcttcttgcatgcatcttgtctttgcttaatttgtagtagaacggatgcagttttgatctctgctgatgatgtgatgattcttcatatgcatatgctctgtacatgtctcttcaaatttgtgtagcaacagtctgtagttctcgttctgctctgaatgaatgcctcttgcatgttgtctttgctagctttgtggtagaaatgtagaatgcagacattgcttccgtcccaaataatctgttccttgcttcgtatatatattgacatgttgtgcatataatctgtgaatgaagttgtgaacaagtcttctttcagaaaaaaaagttgtgaacaagtgcctcacctcacctacaaggctacaaacacaacaacaacagaagctggcctcttcacggagaaccgcgcggggactgctgcagctttctgttgccatattgtttttcacgccaggacaaaatagacggtgcggtttgattcgatcccggttaattctcaatcccttcgtcactatgttccacatggaaccgg

SEQ ID NO: 16 - DNA, 1694 bp, Triticum aestivum
  source 1..1694: mol_type = genomic DNA
atggcccccaccgccctcctcatcgtcgtcctcgccgtcgctgccctccacgcccccgccgcctccgccgcgttgtcccaggagccaccagcgacgccgtgcgcggcagccatcgtgtccttctcgccgtgcctggcgcacgtcgcggtggtggcgccgcccgccctgccctcgcccgcgcccaccagcgcctgctgcgcggcgttcctgcgcgccgtttcctccggggacggcgaaggcggcggaggggagggatgcttctgccacctgctccgcaacccgctcctcctcggcttccccgtcgacgccgcgcgcctcggcactctcctccccacctgcgcctccgcgaaaacctccgccgccacggccgccgaggccgaggccctcttcgccgacaagtgccgaggtgagaaatctgtcccttcgtgctccctatttatgctcgtgcaatatgtatgcttcgatcaattctcgtgcgccatatgcgcgtgctgtcgcgttcctgttgttgatcgccgatcgaagcaaattttactctgcaaagtcctaactactgttgttctcatgatccttgtacaactactttcgtaatggttggttgtaatgcggattactgacgagctctgaatgctactagggatattagcacttgcattactgaactagtggaatgggggagaaacaccgcgggatttttcttttgttattaaggagaagatactgagtggcccagacttacttctgcgtttttgctgcgatttgtcatgattaccgttgatttaagcagtttgttgggttgcttgtttgatagtagtagtaatttcacaaaatattggcgatatttataaatagctaagggcttcgttacttggtggtttctctcaaagagttcacaaaagtcaattattaaattcaattaagggggcaagtattagtagtactggctaactccatttgccatttgccaattgaaacagagctcaagtcactgcctgagatgcattttacacctccatcgccacctcccgcaccaaaactttctccaggtaaatgttctgctgcttgtctaatgattccatagcttgttaaaaaaaatgattccataaatctgtgcccagtaatgctatttcggatttcggttgaatgaaccaattggcatttgggcagacatgtcatatgtcctctcctaccaaatgaaacttgaacttgttttatcttgtggtgctccatccatttcgtgttctatgccacaactgtacaggttcaaatgatagtagaacaactaattttgcatcggatactctctgcagcctctgttctgttcctactaattgcaggaccgagtctgtctttgtgatactatctgctttgcgtgcactgctcttttaggttgcagctgcaccttctattttgttcacctgagcattccattttggtcgactgaacaccgcaagtcctgaatatctttcttgaatcgtcggtcttacttggtgtgcactagttaaactgctgtgtatgcccttgcagctgccgttacagaaccagcgtccccgactccgaagatggaggagcattcgacctcgacgacgcctgtgtcggatgatcggtcgggatccgatgccttgtgtgcctgccgggtcttccttgtggccttggtcttgggagcagcagtcttgatcacgctgcagttctga

SEQ ID NO: 17 - DNA, 2000 bp, Triticum aestivum
  source 1..2000: mol_type = genomic DNA
tgctatgcttaatgcttgtcactagggctcgagtgtcatgatttcagatctaaaccctttatgtttttatcaatatatctgagttcctaatcctatatggtagttatgtgcactttttattgttctgaaccgtagaccccaaggtagcaacatgaaaggggggattaaagggttgaaaacattgatcatgaaagagtccccctctgcctattacatccactgttttgcacatcaacttcaattggttcttacagccgtggcaaaagaaaatgaaccatgttcgtggttctttgatcatgtttcttaattgcttaatattcttggagtttcttgcaagtgacatgacatgctttgagatgttagagctcaaaaggttttggaagcacttgaaatgggtgagattgaaagtggaagtgggctaaatcaagagatgggactagctagacccggcgatactagttggggttctcattttaagaccattatgcacattgttagcatgtatccacaatccttgaagtacttgatgctattggaaaagatccttcacaaaaaggcgagtggacaagaatatgttgagttactcatgcttttgttttcaatcttcgtttgatgctagttattcgtggctatacaaatgagttgtccaaatctttgcaaaagagagatcaagatattgttaatgcaatgacacttgttagtttggcaaatagtagaatgcaacacatgaggtctcatggttgggaagaatttcttgcaaagatgaccttatttggcaacaaaaatgacactgaagttcatttgtggtaggatacttataagcctcatggaagatcacgtcggtattatgaagtacaaacaaatgatgatcattatagaagagaaatgtatcttggtgtcattgattaaaccattcaagagcttgacaatcggtttgatgaggtttacatggagttacttatttgcatgttggctttgaatcccctcaattcatttgcttctgacgatgcaatcaaggtaatgagacttgtcgaattctatcccatggacatatcaagtacatatttgataaggctataatttcaacttgctaattttgttgatgatacgagacaatacgataggtttagaaatgcaagtaatattggtgagctctctattatgcttgttgcaacaatgaaacatgttctttatgatttggtctacttactcatcaaattgatattgattttaccgatggtgactgcgagtgttgaaagagtattttggatcatgaatatagcgaaaagtaagttaaggactagtatgagtgatgaccgcttgaatgattgcttggtgatatttattgagcgggatgtgttcatgaaagcatgtgaagatgacatagttgatgctttcatggcaatgcaaaacgtagagttacctactgttatgattttctacttgtgcttctttcatgtaagactattcgtattgttggagatttgaattgaactatccttgttattctttttgccatgttttgtttgacacacttggattaggtacagaaaaaaatagtgtgcacgcccttcgttttattcctgggttgtcattaggccgtaccaggatcgatcgtgtcctgttgattattaagaccacagtgatctggctcagacggtagtaaactggatcgtgttaatttaggttaagttataatgaaacttcttatcccgcaaaaaagttaatggaacccctttccttcgaaaaaaagataacggacgaatgcccgcagaaaaaccgagtgttgatgacagctagcgacaggcactggatcatcacgagccgtcggatgtgcccaactttgcgccgcctttgttaacggacgggcatgatcgcttttgcaaacccctcagcattacacactcacaccctaaaaccaccaggcatcatcacctcaccacccatcccgccccgatacgagagagagagtggggacgggtgtgctgagagcctgagac

SEQ ID NO: 18 - DNA, 642 bp, Triticum aestivum
  misc_feature 101: note = R = G or A
  source 1..642: mol_type = genomic DNA
atggcccccaccgccctcctcatcgtcgtcctcgccgtcgctgccctccacgcccccgccgcctccgccgcgttgtcccaggagccaccagcgacgccgtrcgcggcagccatcgtgtccttctcgccgtgcctggcgcacgtcgcggtggtggcgccgcccgccctgccctcgcccgcgcccaccagcgcctgctgcgcggcgttcctgcgcgccgtttcctccggggacggcgaaggcggcggaggggagggatgcttctgccacctgctccgcaacccgctcctcctcggcttccccgtcgacgccgcgcgcctcggcactctcctccccacctgcgcctccgcgaaaacctccgccgccacggccgccgaggccgaggccctcttcgccgacaagtgccgagagctcaagtcactgcctgagatgcattttacacctccatcgccacctcccgcaccaaaactttctccagctgccgttacagaaccagcgtccccgactccgaagatggaggagcattcgacctcgacgacgcctgtgtcggatgatcggtcgggatccgatgccttgtgtgcctgccgggtcttccttgtggccttggtcttgggagcagcagtcttgatcacgctgcagttctga

SEQ ID NO: 19 - protein, 213 aa, Triticum aestivum
MAPTALLIVVLAVAALHAPAASAALSQEPPATPCAAAIVSFSPCLAHVAVVAPPALPSPAPTSACCAAFLRAVSSGDGEGGGGEGCFCHLLRNPLLLGFPVDAARLGTLLPTCASAKTSAATAAEAEALFADKCRELKSLPEMHFTPPSPPPAPKLSPAAVTEPASPTPKMEEHSTSTTPVSDDRSGSDALCACRVFLVALVLGAAVLITLQF

SEQ ID NO: 20 - DNA, 1000 bp, Triticum aestivum
  source 1..1000: mol_type = genomic DNA
tggatgttcgacctcgctcttgaaaggtctttgccctcgctcttgatacaatacttgtgcgcgatgctgcgccattcttgattggacctcttgttgcatacggtgaggtgccacgtgatgcagggcatgatattggtatacctgggaactcttcctgaccagtgcagtttgttccgtgcgcgttttgatgacctgacaacgcaagtagtaactgttatccggtgacataatcgtatcatttcaaacatggtacagatttatacaatgcagtttagtttctcttgcgatttattactttctgtgaatctgcatgccattctcttgcactcgctgagagagacggagaactcactggaatttgttttcactatacaaatatgcgcagttgcagcacacgcacgaacaaatacaaagtcacacatcattcgcatttttctttcagagttacacatttgaaatgaaggggaagaaaaacagaagtagcatcgtcagcggaatgaagcattacatgccaattggttgctgatcaggttcagtctgtggactgaatgtattctaggtacattatatgagcaatgacagtagccctgctgccacgatcgaggaggcgagaactacggaggatatcccacgacagagcttccggtgggcgctggacgggttgaacatgctgtcacgcgggtacatctgccccggcgtccccactatgccaaacaccggcgccggctgcacgtacgggtacacgtacggctcagccggcggtggaaggtagtacacgggcgttggttccggctccgacggcggtgggtagtagtagtacactggcgtcggcacaggctccggcggaggcgggcagtgctgtggctttggcttcggctcgggctccggctccggctgtggctcaggctcaggctttggctccggctctggttccggctgtggctcaggctttggctctggctctggctctggctccgacggtggcgggtaaggcgctggg

SEQ ID NO: 21 - DNA, 1629 bp, Triticum aestivum
  source 1..1629: mol_type = genomic DNA
atgccccccaccgccctcctcctcgtcgtcctcgccgtcgccgccctccacgcccctgccgcctccgccgcgttgtcccaggagccaccagcgacgccgtgcgcggcggccatcgtgtccttctcgccgtgcctggcgccgtcgcggtggtggcgccgcccgccctgccctctcccgcgcccaccagcgcctgctgcgcggcgttcctgcgcgccgtttccgccggggacggcgaaggcggcggaggggagggatgcttctgccacctgctccgcgacccgctcctcctcggcttccccgtcgacgccgcgcgcctcggcgctctcctccccacctgcgcctccgcgaaaacctccgccgccacggccgtcgaggctgaggccctcttcgccgacaagtgccgaggtgagaaatctgtcccttcgtgctccctatttatgctcgtgcaatatgcatgcttcgatcaattctcgtgcgccatatgcgcgagctgtcgcgttcctgttgttgatcgccgatcgaaacaaatcttactctgcaaagccctaactactgttgttctcatgatccttgtacaactactttcgtaatggttggttgtaatgcggattactgacgagctctggatgctactagggatattagtacttgcatttctgaactagttgaatgggggagaaacaccgcgggatttttcttttgtggcccagacttacttctgcatttttgctgcgatttgtcatgattaccgttcagttaagcagtttgttgggttgcttgtttgatagtagtaatttcacaaaatattggcgatatttataaatagctaaaggcttcgttacttggtggtttctctcaaagagttcacaaaagtcaatcattaaatcaattaagggggcaagtattagtagtactggctaactccatttgccattgaaacagagctcaagtcactgcctgagatgcattttacacctccatcgccacctcccgcaccaaaactttctccaggtaaacgttctccctcttgtctaatgatttgataaatctgtgcccagtctattcgggtttcggttgaataaaccaattggcatttgggcagcatgttatatggtctctcctcatcccaaataaaacttgaacttgttttatcttgccgtgctccgtccattttgtgctctatgccgcaattgcacaggttcaatgatagtagaacacctagttttgcatcgcatactctctgcagcctctgttctgttcctgctaattgcagtatcgtgtctgtctttgtgatactatctcctttgcatgcactgcacttttaggttgcagttgcacgtttgattctagtttgttcacctgagcattccattttggtcgactgaacaccgcaagtcctgaatatctttcttgaatcgtcggtcgtacttcgtgtgcactagttaaactgctgtctatgcccttgcagctgccgttccagaaccagcgtccccgaagatggaggagcattcgacctcagcgacgcctgtgccggatgatcggtcgggatccgatgccttgtgtgcctgccgggtcttccttgtggccttggtcttgggagcagcagtcttgatcacgctgcagttctga

SEQ ID NO: 22 - DNA, 2000 bp, Triticum aestivum
  source 1..2000: mol_type = genomic DNA
aacatccgtacaagttacatgaccagaaacacgtatttttttacaagtttgtacaccgcgagccgtcctatgcggattttttgttgaaaaggaccatctgggcgaatgaattgacaaaaaagaccacctgccgataaatttaacaaaaaggaccccctcggctgtggcggcaggtgcggcaggcgacacgtggcacctgccgccacacagcaaggcggcagcactgtaccaacgggaatcggcgctggtgacggaacggcagtggcgtgtggggcccggccgccaagggacgtggcagcagtactgtacctggggcgcagccgcctggcgggctggcggcagccctgttcatggcgctgctgctctactgcacgctacgctgattgctaccgctgctgcatgcgcttttagccggccactgctgctgattggtactgttactgcatgcgtttttagccggccactcatgctgcatgcaagatgttattttcttgacatgcacgatgttgttaattttgtcattaacctttgtgttagagcatgaattagagttaggatgcagaagatgttttagttgaagaaaagatagatcaaggtaagatatacccagtaattagtaattgaatattgtattattcggtttgactaataatcaagtggaaaaacattttaacatccgtatttatgtttgaaataatcattttttattaatatcattcggttcgactggaaacataataatatgatgataacatttaaatagtatgacttaataaataaccgtatttccgttagcaataatcggtttgcttttagtatcatttgcttagaatttaaacataatagtttgtttacggataataatacgacaaatattcaaagcagcggtaacatcattcgcaacagtacatcaatataaaaaatattcataatattacacggcgtcatttatacctcagcttcgatatggatgtcgttccgaataaaattaaccgtcaaaagggagatcgaggatcgaaacgcaatttcttcaactacgtcatctcctccttgtttgttactcagatgataccttttttacctaattacatacatcattaaggatattagcattttataatatacaaaacttagattattaacaaaataaactacggtttcgtaactatacaacttattaatatatttttttaccacatgaaataacaacgcattaaatcatccaatgaccaataacaacacaaactttttcttacttaccatattgataaattcttgcatgtcaaactaaatcgatacattttaaaggcctaaagctgatctacaatactgttaaatatttgctttactaattcctgctctaacacaaaggttaatgatagaattaacaacatcgtgcatgtcaagaaaataacatcttgcatgcagcagcagtggccggctaaaagcgcatgcagtagcagtaccaatcagcaacagtggccggctaaaagcgcatgcagcagtagcaatcaacgtagtaatagagcagcagggccatgaacagggctgccgccagcccgcgaggcggccgctccccaggtacagtactgccgccacgccccttggcggctgggccccacacgccgctgccgttccgtcgccagcgcccattcccgttggtacagtgctgccgcctcgctgtgtggcggcaggtgccacgtgtcgcctgccgcacctgccgccacagccgagggggtcctttttgttaaatttatcggcatgtggtcctttttgtcaattcattcgcgcatgtggtccttttcaccaaaaaatctcctatgcgcccacctttgcgccatcttgttaaacccctcagtattacactttttttttgcggggacagtattacacactcacgccctaaaaccaccaggcatcatcacctcaccacccatcccgccccaatacgagagaaagagagagagagagtggggacgggtgtgctgagagcctgagac

SEQ ID NO: 23 - DNA, 635 bp, Triticum aestivum
  source 1..635: mol_type = genomic DNA
atgccccccaccgccctcctcctcgtcgtcctcgccgtcgccgccctccacgcccctgccgcctccgccgcgttgtcccaggagccaccagcgacgccgtgcgcggcggccatcgtgtccttctcgccgtgcctggcgccgtcgcggtggtggcgccgcccgccctgccctctcccgcgcccaccagcgcctgctgcgcggcgttcctgcgcgccgtttccgccggggacggcgaaggcggcggaggggagggatgcttctgccacctgctccgcgacccgctcctcctcggcttccccgtcgacgccgcgcgcctcggcgctctcctccccacctgcgcctccgcgaaaacctccgccgccacggccgtcgaggctgaggccctcttcgccgacaagtgccgagagctcaagtcactgcctgagatgcattttacacctccatcgccacctcccgcaccaaaactttctccagctgccgttccagaaccagcgtccccgaagatggaggagcattcgacctcagcgacgcctgtgccggatgatcggtcgggatccgatgccttgtgtgcctgccgggtcttccttgtggccttggtcttgggagcagcagtcttgatcacgctgcagttctga

SEQ ID NO: 24 - DNA, 324 bp, Triticum aestivum
  source 1..324: mol_type = genomic DNA
atggcccccaccgccctcctcctcgtggtcctcgccgtcgccgccctccacgcccctgccgcctccgccgcgttgtcccaggagccaccagcgatgcgggatttttcttttagctcaagtcactgcctgagatgcattttacacctccatcgccacctcccgcaccaaaactttctccagctgccgttccagaaccagcgtccccgaagatggaggagcattcgacctcagcgacgcctgtgccggatgatcggtcgggatccgatgccttgtgtgcctgccgggtcttccttgtggccttggtcttgggagcagcagtcttga

SEQ ID NO: 25 - protein, 205 aa, Triticum aestivum
MPPTALLLVVLAVAALHAPAASAALSQEPPATPCAAAIVSFSPCLAPSRWWRRPPCPLPRPPAPAARRSCAPFPPGTAKAAEGRDASATCSATRSSSASPSTPRASALSSPPAPPRKPPPPRPSRLRPSSPTSAESSSHCLRCILHLHRHLPHQNFLQLPFQNQRPRRWRSIRPQRRLCRMIGRDPMPCVPAGSSLWPWSWEQQS

SEQ ID NO: 26 - protein, 107 aa, Triticum aestivum
MAPTALLLVVLAVAALHAPAASAALSQEPPAMRDFSFSSSHCLRCILHLHRHLPHQNFLQLPFQNQRPRRWRSIRPQRRLCRMIGRDPMPCVPAGSSLWPWSWEQQS

SEQ ID NO: 27 - DNA, 1000 bp, Triticum aestivum
  source 1..1000: mol_type = genomic DNA
tggatgttcgacctcgctcttgatacaatacctgtgcattgctgcgtcattcttgattggatcctcttgttgcatacggtgaagtgccacgtgatgcagcgcgtgatacctgagaactcttcctgactagtgcagtttgttctgtgcgcgttttggatgacctgacaacgcaagtaactgttatccggtgacatactcgtatcgtttcaaacatggtatagatttatacaatgcagtctagtttctcttgcgattttctttctgtgaatctgcatgccatttccttgcgctcgctgagagagacgagaactcaggagcacagcacaggagcaaactctcatgctttcatggtgcatgaccttgctgcgctagctgcgccggtgaccactccggtcccggcggccaccagtccccccccccccccccccccccccccccccccccccccccccccccggggcccggcgtccccctccgcttcccatggcctcgcaagtctctctctcggcggattcgtcttcctcgacctcaccttccctgcctagtgccccgctgcgctccatcgtcgtcgttcctgatgtcctgggcacgcgcttcggccccatcgcccagggcgctctcggcccatcgcgggccccggcggcggcctcttgagcgtggcaaaccaagatggggcgtcgcccccgcagcccggcttctccgcctcgccccccccccccccccccccccccccccccccccccccccccccccccccccccgcgtcgccatggcaacaccctgcccacctccaggccgccgaggaccaacatccctgcggtcctccacggttgctgctataactgcggcgatgacgggcacatctctgtggactgctggaaggagaccgtctgcatgcgctatgggggtgcgggccacatcgccagggattgcgcctcgccccgcgactcctctccggtggacaggcatttgccgcgctggtcggcggccccggctcagc

SEQ ID NO: 28 - DNA, 1665 bp, Triticum aestivum
  misc_feature 25: note = S = C or G
  source 1..1665: mol_type = genomic DNA
atggcccccaccgccctcctcctcstcgtcctcgccgtcgccgccctccacgcccccgccgccaacgccgcgttgtcccaggagccaccagcgaccccgtgcgcggcggccatcgtgtccttctcgccgtgcctggcgcacgtcgcggtggtggcgccgcccgccctgccctcgcccgcgcccaccagcgcctgctgcgcggcgttcctgcgcgccgtttccgccggggacggcgaaggcggcggaggggagggatgcctctgccacctgctccgcgacccgctcctcctcggcttccccgtcgacgccgcgcgcctcggcgctctcctccccacctgcgcctccgcgaaaacctccgccgccacggccgtcgaggccgaggccctcttcgccgacaagtgccgaggtgagaaatctgtcccttcgtgctccctatttatgctcgtgcaatatgtatgctccgatcaattctcgtgcgccatatgcgcgtgctgtcgcgttcctgttgttgatcgccgaccgaaacaaattttactctgcaaagtcctaactactgttgttctcattatcctcgtacaactactttcgtaatggttggttgtaatgcggattactgacgagctctggatgctactagggatattagtacttgcatttctgaactagtggatgggggagaaagcgggatttttcttttgtggcccagacttacttctgcatttttgctgcgatttgtcatgattaccgttcagttaagcagtttgctgggttgcctgtttgatagtagtaatttcacaaaatattggcgatatttataaatagctaagggcttcgttacttggtggtttctctcaaagagttcacaaaagtcaatcattaaattcaattaagggggcaagtattagtagtactggctaactccatttgccatttgccaattgaaacagagctcaagtcactgcctgagatgcattttacacctccatcgccacctcccgcaccaaaactttctccaggtaaatgttctgctgcttgtctaatgattccatagctcgtcaaaaaaaaaatgattccataaatctgtgcccagtaatgctatttcggatttcggttgaatgaaccaattggcatttgggcagacatgtcatatttctttcctcccaaatgaaacttgaacttgttttatcttgtggtgctccatccatttcgtgctctatgccgcaactgtacaggttgaaatgatagtagaacatctaattttgcatcggatactctctgcagcctctgttctgtttctactaattgcaggatcgagtctgtctttgtgatactatctgctttgcgtgcactgctcttttaggttgcagctgcaccttctattttgttcacctgagcattccattttggtcgactgaacaccgcaagtcctgaatatctttcttgaatcgtcggtcgtacttcgtgtgcactagttaaactgctgtgtatgcccttgcagctgccgttccagaaccagcgtccccgactccgaagatggaggagcattcgacctcgacgacgtccatgtcggatgatcggtcgggatctgatgccttgtgtgcctgccgggtcttccttgtggccttggtcttgggagcagcagtcttgatcacgctgcagttctga

SEQ ID NO: 29 - DNA, 1666 bp, Triticum aestivum
  source 1..1666: mol_type = genomic DNA
atggcccccaccgccctcctcctcgtcgtcctcgccgtcgccgccctccacgcccccgccgccaacgccgcgttgtccccaggagccaccagcgaccccgtgcgcggcggccatcgtgtccttctcgccgtgcctggcgcacgtcgcggtggtggcgccgcccgccctgccctcgcccgcgcccaccagcgcctgctgcgcggcgttcctgcgcgccgtttccgccggggacggcgaaggcggcggaggggagggatgcctctgccacctgctccgcgacccgctcctcctcggcttccccgtcgacgccgcgcgcctcggcgctctcctccccacctgcgcctccgcgaaaacctccgccgccacggccgtcgaggccgaggccctcttcgccgacaagtgccgaggtgagaaatctgtcccttcgtgctccctatttatgctcgtgcaatatgtatgctccgatcaattctcgtgcgccatatgcgcgtgctgtcgcgttcctgttgttgatcgccgaccgaaacaaattttactctgcaaagtcctaactactgttgttctcattatcctcgtacaactactttcgtaatggttggttgtaatgcggattactgacgagctctggatgctactagggatattagtacttgcatttctgaactagtggatgggggagaaagcgggatttttcttttgtggcccagacttacttctgcatttttgctgcgatttgtcatgattaccgttcagttaagcagtttgctgggttgcctgtttgatagtagtaatttcacaaaatattggcgatatttataaatagctaagggcttcgttacttggtggtttctctcaaagagttcacaaaagtcaatcattaaattcaattaagggggcaagtattagtagtactggctaactccatttgccatttgccaattgaaacagagctcaagtcactgcctgagatgcattttacacctccatcgccacctcccgcaccaaaactttctccaggtaaatgttctgctgcttgtctaatgattccatagctcgtcaaaaaaaaaatgattccataaatctgtgcccagtaatgctatttcggatttcggttgaatgaaccaattggcatttgggcagacatgtcatatttctttcctcccaaatgaaacttgaacttgttttatcttgtggtgctccatccatttcgtgctctatgccgcaactgtacaggttgaaatgatagtagaacatctaattttgcatcggatactctctgcagcctctgttctgtttctactaattgcaggatcgagtctgtctttgtgatactatctgctttgcgtgcactgctcttttaggttgcagctgcaccttctattttgttcacctgagcattccattttggtcgactgaacaccgcaagtcctgaatatctttcttgaatcgtcggtcgtacttcgtgtgcactagttaaactgctgtgtatgcccttgcagctgccgttccagaaccagcgtccccgactccgaagatggaggagcattcgacctcgacgacgtccatgtcggatgatcggtcgggatctgatgccttgtgtgcctgccgggtcttccttgtggccttggtcttgggagcagcagtcttgatcacgctgcagttctga

SEQ ID NO: 30 - DNA, 2000 bp, Triticum aestivum
  source 1..2000: mol_type = genomic DNA
caatgtgggcacagatggcttcctcctttgcggtcgcccgcgtcgccgccatgtcggtggcgagacgggtctcgaaggcccttatcctctccttcccacggcgggcacactggtcccggtgggactcatagtccacccgatcgatgatggggaaggagagattttccggctaccgggtctgcgatgatccagcccaaaggattcaagcgccattgagtcgatgctacggagtcgcgccggacagatcctgccgtcggccaggcgctctcctcccgggagcggcggagtgcaatgaggacaaccattgcttcctccaacccgcacgggatgacaccctagtcgacataatcggactggtcggattcagatccactgcccgccatgccggcgacggccggagaacgccggaggtgagcttgtgtggcgcaggagagtgtagtgaagtggctagggtttgcttcggcgagcggatggggaggaatatatgtggggacgggtgggccaccgcgggcctgttctgacatggcggacgcgctagggcgcgcctgggtgcccccatatccgttctatatttgggctggatatgaggggtgtcggtcagcccgagcgtatgaggctcgtttgaggcgcccgcctgggtcgaaatttcgtgaccggtcagtgaccaggcgacccgtccggacgtatgaggcgggtttggagtgccggactgcagattctctaattttctacttgtgcttcttttcatgtaagactattcgtattctaattgagcatgtttgggatatattttgtttactaagttgtgagattctaattgagctattcatgtgattcttttgccatattttgtttgacacttggattaggtagaaaaaaattagagtatgcacttccttcattttactcctgggtccgcactgagtcgtagttaggttcggtcttcttgtgttgatgttaagaccacagtctggttcggatttggctcagatgggcagtaaactggatcgtgtttatccaagtcaagttggtaccattaaaagaaatatactcatttgtttggaaccgctactcaagagaaaggcgaagtgagtggcgactcagtttctctccagtttccccgacaccgttgtgggttccctttcccatcttcggccgcttcggcagcggaagggcaggggaaccccggatctggttgtctagtgtgtagatagggtgtgggtatgtgtgcctggcggcaacgtcttcatgggagtggtggcgctatgcatggcaataaagtatccccgatcctccctcctctcacggcgatcttggtatcggcgttgatggatcagtgggagtttggtttggtgctgcagcggcggcaggagtagcacctctggctgtttgggctctcggttctttggcttcggatccggcgaccatgacctcgtctccgagatgtttggcagtgatgacttcccgtccgcgtgattcttgaaggttcgccgcaatctggcagcggggttgccggagcggctacgacggcacgccatcggaccatcttggaggttggagaagtgtgacatcacagggacctgattgcagtttctttctttttcatgggtgtcttgtactgctggtttgtgcttaattgatcctagtgttttttttaaatccaggtcaagttaatgggtccctttaccccgtcaaaaaagttagttaatggaacccatttcctccggaaaaaaaagataacggacggaatgcccgcagaaaaatcaagtgttgatgacggctagcgattggcactagatcaccacgagccatccgatgcacccaactttgcgccgccttgttaacggacgggcatgatcgcttttgcaaacccctcggtatcacacactcacgccctaaaaccaccaggcatcatcacctcaccacccatcccgccccgatacgagagagggaggagagagagagagagagtggggacgggtgtgctgagagcctgagac

SEQ ID NO: 31 - DNA, 642 bp, Triticum aestivum
  misc_feature 25: note = S = G or C
  source 1..642: mol_type = genomic DNA
atggcccccaccgccctcctcctcstcgtcctcgccgtcgccgccctccacgcccccgccgccaacgccgcgttgtcccaggagccaccagcgaccccgtgcgcggcggccatcgtgtccttctcgccgtgcctggcgcacgtcgcggtggtggcgccgcccgccctgccctcgcccgcgcccaccagcgcctgctgcgcggcgttcctgcgcgccgtttccgccggggacggcgaaggcggcggaggggagggatgcctctgccacctgctccgcgacccgctcctcctcggcttccccgtcgacgccgcgcgcctcggcgctctcctccccacctgcgcctccgcgaaaacctccgccgccacggccgtcgaggccgaggccctcttcgccgacaagtgccgagagctcaagtcactgcctgagatgcattttacacctccatcgccacctcccgcaccaaaactttctccagctgccgttccagaaccagcgtccccgactccgaagatggaggagcattcgacctcgacgacgtccatgtcggatgatcggtcgggatctgatgccttgtgtgcctgccgggtcttccttgtggccttggtcttgggagcagcagtcttgatcacgctgcagttctga

SEQ ID NO: 32 - DNA, 643 bp, Triticum aestivum
  source 1..643: mol_type = genomic DNA
atggcccccaccgccctcctcctcstcgtcctcgccgtcgccgccctccacgcccccgccgccaacgccgcgttgtccccaggagccaccagcgaccccgtgcgcggcggccatcgtgtccttctcgccgtgcctggcgcacgtcgcggtggtggcgccgcccgccctgccctcgcccgcgcccaccagcgcctgctgcgcggcgttcctgcgcgccgtttccgccggggacggcgaaggcggcggaggggagggatgcctctgccacctgctccgcgacccgctcctcctcggcttccccgtcgacgccgcgcgcctcggcgctctcctccccacctgcgcctccgcgaaaacctccgccgccacggccgtcgaggccgaggccctcttcgccgacaagtgccgagagctcaagtcactgcctgagatgcattttacacctccatcgccacctcccgcaccaaaactttctccagctgccgttccagaaccagcgtccccgactccgaagatggaggagcattcgacctcgacgacgtccatgtcggatgatcggtcgggatctgatgccttgtgtgcctgccgggtcttccttgtggccttggtcttgggagcagcagtcttgatcacgctgcagttctga

SEQ ID NO: 33 - protein, 213 aa, Triticum aestivum
MAPTALLLVVLAVAALHAPAANAALSQEPPATPCAAAIVSFSPCLAHVAVVAPPALPSPAPTSACCAAFLRAVSAGDGEGGGGEGCLCHLLRDPLLLGFPVDAARLGALLPTCASAKTSAATAVEAEALFADKCRELKSLPEMHFTPPSPPPAPKLSPAAVPEPASPTPKMEEHSTSTTSMSDDRSGSDALCACRVFLVALVLGAAVLITLQF

SEQ ID NO: 34 - protein, 141 aa, Triticum aestivum
MAPTALLLVVLAVAALHAPAANAALSPGATSDPVRGGHRVLLAVPGARRGGGAARPALARAHQRLLRGVPARRFRRGRRRRRRGGMPLPPAPRPAPPRLPRRRRAPRRSPPHLRLRENLRRHGRRGRGPLRRQVPRAQVTA

SEQ ID NO: 35 - DNA, 600 bp, Triticum aestivum
  source 1..600: mol_type = genomic DNA
tggatgttcgacctcgctcttgaaaggtctttgccctcgctcttgatacaatacttgtgcgatttttttttagaaaaggaggacgacccctggcctctctgcatctggacgatgcatacggccattttattaattatattggtatacctgagaactcttcctgatcagtgcagtttgttccgtgcgcattttgatgacctgacagcgcaagtaattgttatccggtgacataatcgtataatttcaaacatggtacagatttatacaatgcagtttagtttctcttgcgatttattactttctgtgaatctgcatgccattctcttgcgctcgctgagagagacggagaactcactggaatttattttcactatacaaatatgcgcatttgcaccacacgcacgaacaaattcaaagttcaacatcagtcgcatttttctttcagagttacacatttgaaatgaaggggaaaaaaatgaacaggagtagcatcgtcagcggaatgaagcattacatacaaattggttgctgatcaggttcagtctgtggactgaatgttttctgggtacattatatgagcaatgtcagtagccctgctgc

SEQ ID NO: 36 - DNA, 1716 bp, Hordeum vulgare
  source 1..1716: mol_type = genomic DNA
atggccccccccgccctcctcctcctcgtcctcgccgtcaccgccctccacgcccacgccgcctccgccgcgttgtcccaggagccaccagcgacgccgtgcgcggcagccatcgtgtccttctcgccgtgcctggcgcacgtcgccgtggtcgcgccgcccgccctgccctcgtccgcgcccaccagcgcctgctgcgcggcgttcctgcgcgccgtttcctccggggacggcgaaggcgctggcggaggggagggatgcttctgccacctgctccgcgacccgctcctcctcggcttccccgtcgacgccgcgcgcctcggcgctctcctccccacctgcgcctccgcgaaaacctccgccgccacggccgtggaggccgaggccctcttcgccgacaagtgccgaggtgataaatctgtcccttcgtgctccctatatttgctcgtgcgacatgtatgcttcgatcgattctcgtgcgtcatatgcgcgtcttgtcgcgttcctgttgttgatcaccgatcgaaacaaattttactctgcaaacttctaactaatgttgttctcatgatccttgcacaaccacttccgtaatggttgtttgtaatgcggattacaaacgagctctgggtgctactagggatactaccacttgcattcctgaactagtggaaagggggagagacaccgtgcgtctttttcctttgtaaagcagaaaatacaaaatagtgagaggcccagaattacttttgcatttgcgctacgatttgtcatgattaccatccagttaagctgtttgttgggttgcttgttttacagtaatttcacaaaattttggcgacatttataaatagctaagggcgtcattacttggtggtttctatcaacgagttcagaaaagtcaatattaattcagagggcaaatattagtagtactggctaactccatttgctatctgccaattgaaacagagctcaagtcactgcctgagatgcattttacacctccatcgccacctcccgcacccaaactttctccaggtaagcgttctcctgcttgtctaatgattccataaatctgtgtgcagtaatgctattcggatttcggttgaatgaaccaattggaatttgggcagacatgtcatatgttgaatgtatcaattggcatttgggcagacatgtcatatgttggatgaaacaattggcatttgggcagacatgccatatgttctctcctcctcccaaattaaaccatccatttcgtgctatatgccgcaactgtacaggatcaaatgatagtggaacatctgattttgcatcgcatactctctgcagcctctgttctgttcctactaatcgcaggattgagtctgtctttgtgataccatctcctttgcgtgcactgctcttttagtttgcagctgcacgttctagtttgttcacctgagcatgccattttggtcgtctgaacaccgcaagtcctgaatgtctttcttgaatcatcggtcgtacttggtgcgcactagttaaactgctgtgtatgcccttgcagctgccgttccagaaccagcgttcccggctccgaaaatggaggagcattcgagctcgacgcctgcgccgggtgatcggtcgggatccgatgccgtgtgtgcatgccgggttttccttgtggccttggtcttgggagcagcagtcttgatcacgctgaacttctga

SEQ ID NO: 37 - DNA, 2000 bp, Hordeum vulgare
  source 1..2000: mol_type = genomic DNA
agcaccgtagcagtagcaagggtgcccgcgctattgatacggggaaaacccgcgctactgctagactttccctagtagtgaccgggcacaccgttgtgtctcttcttggatggtacttgtgatgtacatcatgagggctatgtaccaacaagtggactctttgtttagttttgtcaaggaaagacattgttcgggtgcttacctcaggtatgtaatgtaggacgagtcgtggcttgacacaaaagttccctaaatcttgtggataaagtgtgaaacctctacagaatgtaaactatttgaatagtcatgtctgcggtcaaggacagttgggtgttgcttcttaaactacatccaatgttttacaaaacttgatgtgcgtgtgggaaagaaatacttgagctaagtcaaatgacttggtgagatgatttaaaaatggagttgttccaaacttattttatattgatatatgtaacagttcatatggatactcataaccacatgatgcatgttttataagttgttagaacatgtcattgcagtaaaaattagctttctgcaaaattacctacttagtgttataccatgcatttgttttaccttgttctaaacatgagttggttgcaagtacattaaaagtacttattggcttgccactggttattttattggtcaagcatgaaagaacgagaatatgatgaagaattccttggtgacgatcatgcgaattaggtcgtttcctagtcacatgagatatatgttattaataattaccaactccaccaagggattggatgcacttttagctgcttgagtcctaccactccgtccgtgagatccgcttcgactgttggcagtaccgctagcgttaggcgacgaggagagttcaacgaggcgacatatgaggtgtggggcaagagcgacgacgaggaggacatcactcgcttcaccaaggccgcttcccgttgccacgaccacgaagctggcacgtccgcgagcgccgctggcagtaaggaagagtggtgcatggtaagtcgctatcgctcgggtctaggaaggacaccactcttcccatccacctgaaggcaagcttggcgggttaataaacgtcggggttagctgctcaagctgcgatggtggaggctacagaggcaaagctggagaaggcgaaggcaagtgtcatagcttcaatttccagggctcatggtggtctggtagggaacggatgctaacgatcattttgattaggaatcatacctcgtgagttccatttttagttgaaatatgtctaaaatgtaatgacttttatcatgttcatatgaaatccgtcgtgtttacataaatcgctttttatttatttcaaaatccatcaattttgcacaaattccgtccgttagctcaattattatccattaattgttagaatatatatggacaatattgaatgacggacttcgacatcagtaaccgtggatttattccgcggatatatgtccatgaaatttgaccacattgtagtcgtgtacaggaacggatttaggaggagggggggggggctagaccccctaacaaattgattcttcacctatgattatggttgttataacaaaaaaaattcacttcacatgaatttttccttttcgatgaacaaacttgccctttcctatgcatgaatgttggctccgtcgtgaacgtgtgtggcccaaattggttgtaaatgtctgtaaactggattgcgttgatccaggttaggttgaacccctctccttgtaaacgagtgttgatgacggctggcgacaggcagtggatcatcacgagccgtccgacgtaggcgactttgcgccgcctctgttaacagacgggcacgatcgcttttgcaaacccctcggttttacacactcacgccctaaaaccaccaaggcatcatcacccaccacccacgcccctatacgagagagagagagagagagagtgtggggacgggcgtgctgagagcctgagac

SEQ ID NO: 38 - DNA, 642 bp, Hordeum vulgare
  source 1..642: mol_type = genomic DNA
atggccccccccgccctcctcctcctcgtcctcgccgtcaccgccctccacgcccacgccgcctccgccgcgttgtcccaggagccaccagcgacgccgtgcgcggcagccatcgtgtccttctcgccgtgcctggcgcacgtcgccgtggtcgcgccgcccgccctgccctcgtccgcgcccaccagcgcctgctgcgcggcgttcctgcgcgccgtttcctccggggacggcgaaggcgctggcggaggggagggatgcttctgccacctgctccgcgacccgctcctcctcggcttccccgtcgacgccgcgcgcctcggcgctctcctccccacctgcgcctccgcgaaaacctccgccgccacggccgtggaggccgaggccctcttcgccgacaagtgccgagagctcaagtcactgcctgagatgcattttacacctccatcgccacctcccgcacccaaactttctccagctgccgttccagaaccagcgttcccggctccgaaaatggaggagcattcgagctcgacgcctgcgccgggtgatcggtcgggatccgatgccgtgtgtgcatgccgggttttccttgtggccttggtcttgggagcagcagtcttgatcacgctgaacttctga

SEQ ID NO: 39 - protein, 213 aa, Hordeum vulgare
MAPPALLLLVLAVTALHAHAASAALSQEPPATPCAAAIVSFSPCLAHVAVVAPPALPSSAPTSACCAAFLRAVSSGDGEGAGGGEGCFCHLLRDPLLLGFPVDAARLGALLPTCASAKTSAATAVEAEALFADKCRELKSLPEMHFTPPSPPPAPKLSPAAVPEPAFPAPKMEEHSSSTPAPGDRSGSDAVCACRVFLVALVLGAAVLITLNF

SEQ ID NO: 40 - DNA, 1000 bp, Hordeum vulgare
  source 1..1000: mol_type = genomic DNA
tggatgttggacggacctcgctcttgaaaggtctttgccctcgctcttgatacaatacttgtgcgttgctgcgtcgttcttgattggatcctcttgttgcatacggtgaggtaccacgtgatgcagggcatgatattgatacttgaaaactcttcctgacaagtgcagtttgttctgtgcgcgttttgatgacctgacaacgcaagtaactgttatccttgtggcatactcgtatcgttccaaacatggtacagatttatacaatgcagtttagtttctcttgcaattgtccttctatgaatctgcatgccgttttcttgggatcactggaattttattttcaccatacaaatatgcttaattgcaccacacgcatgaacaaaattcagtgttacacatttgaaatgaagcaaacaaaatgaaaaataaacagggttagcatcgtcagcggaatgaagcattacatgccaattggttgctgatcaggttcagtctgtggacagaatggtttgtacgaaagctacacgagcattgacagtagccctgctgccacaatcgaggaggcgaggattgcagaagatatcccacgacacagcttccggtgggcgctggacgggtagaacatgctgtcgcgcgggtacatctgccccggcgtgcccactatgccaaacaccggcgccggctgcacgtacgggtacacgtacggctcagccggcggtggaaggtagtacacgggcgttggttccggctccgacggcggtgggtagtagtacgccggcgtcggcacagcctccggcggaggcgggcagtgctgtgtcgctggctccggctgtggctccggccgtggctccggttccggctgtggctccggccgtggctccggctctggctcaggctccggctctgacggtggcgggtacttgtactcgtgctgaggaggcgctgggtggtgcgacggcattgccggcggcagcgggtgcggatccaggtcgcagacgca

SEQ ID NO: 41 - DNA, 1568 bp, Brachypodium distachyon
  source 1..1568: mol_type = genomic DNA
atgggacccaccgccctccacgtcgtcatcgccgtcgcggccctcctcctcgccgccgccgcctccgccgcgtcgtcccaggagcctccggcgacgccgtgcgcggcggccatcgtggccttctcgccgtgcctggcgcacgtcgcggtggtggcgccgcccgccgtggccgcgcccgcgcccaccggcgcctgctgcgcggcgttcctgcgcgccgtctcggccggggacggcgaaggcggcggcggggagggatgcttctgccacctcgtccgcgacccgctcctcttcggcttccccgtcgacgtcgggcgcctcggcgcgctcctccccacctgcgcctccgccaacgcctccgcagccaccaccgtcgaggccgaggccctcttcgccgacaagtgccgaggtgacaaagtccgtcctcccttcctgctgatatgctccccatacatgttcgtgtgatatgtatgcttcgatcgattctcgttctcgctatatatgcgcgtgttcttcgctcttgattaccgctccacttaaaagttacttcacgatccatctccatagttcctgtgctcgaaatccttgtggaaccacatttgtaattaatagttggtgggagatgcggagtatcgacgagctctagatgttattgggatgttattatttgtgcattgagaagttaatcagttagtttgctcgtaccaattctgttattaatatagttctcgtttattagacaagaatttcataaatattgggtgatctttgataattatttatttaaggtgtcatttcttggtggtctcttttaagaagttcatggaagtaaaatgctagaaaaatcagtggcacaggtgttactattctctaattcgaattgcaaattgaagcagagctcaagtcactgcctgagatgcatttgtcacctccatcaccacctcctgcgccaaaactttctccaggtaaacattttttctgcttgtctaatgattcgttaatctgtgtccagtaatcccattcagaggcacaaaaaggaattggtacttcggctcatgcgccgtatgatttctcgcaaacttaatttgttaaactctgcaatgttctatccatttcgtgttgtatgaagataactgcagaagtacagattcaaacttaactgtatcagtttaaaaaaaaaacttaactctatggtaaaccaaatagtttggctactagttgctgcatcgagtccctctgttatgttcagttgcactgctctgcaacgttgtagctttccagtttgtcaacctgagtaatcgctgatcgatcatccattcaggtcatttgaacgctgcaatgtctttttggaatcgtcagtcatgtacttcgtgtgcacttgttaatctgctgtgcatgtctctgcagctgccgtcccaggaccagcgtcgtccccgaaggtggaggcggcgcaatcgacgacgtccacgccacgtgatcggtcaggatcggatggcttgtgtgccttccgggtttcgctcatggcgttggtcttcacagcagcagtcttgatcatgttgcagctctggtag

SEQ ID NO: 42 - DNA, 2000 bp, Brachypodium distachyon
  source 1..2000: mol_type = genomic DNA
agatttcatcccacctttgtggtgtggtgagttggagaatagagcgagccttcatggcgtctctaactttgtggtagagtactcatccaaacggagacgtacaccgatcccaataggtgaaactccgatgaaatcttcgtctccacgtgtggtatcatttcctattcacccccgctctagtcgatcataccgatctttcaataagcgacgtggatttgtatagattcttatgcaaatccacttcacttatttcggacggaggggtatattcacatagatcaacaacacatacagctcgtcgtgcaccatgagacacaaactcaagattaactagcagcacgtactccactgctcgtacactccagcaagctagcatgtactcactccttctcatgttaagtgactcaaatttgtctaaatatgaatgtatctatgtctaaaaaatgtctaaatacataaatacatgtaatgaaaagtcagttaatatggaacggagagagtacaatgcaattgtgatccagtccagcaaactacagtactcaactgctaggtcgtacagtccagcagtacatgcaagttggggagaaacagtggctgctgtcccttccttcgtgtgatttagagatccacgtatcgccccgtgcactttacgtttaaacactgagcttcgccgtttgtgcgctgtgagttgatcgacggctccagattagtggagcaagacgctcgggagcagttgagactttctctattttgaaatattattctcttgacatatcaaaattttgttgttatatcactgaaatttcattatcacatcactatttcattactatttcaatcaataacactacggttgtatgttggtaaattcaagctcagaattgagatttggaattggggttcctgaattctgttgtttggatgtccgtggaattcatagttggaatttgagtcttattcattggtattcaactacaaccgaaagagacctctctcaataccgagccagacgcagtgctcctgctaaacgcagccatggtcatgggaggctccctgccgaccaccaccgctacccgcagccgcctcgtcgccggccatctccgcggcctgcagccaccgcgctgccggacatcttcgtggcccgtggccactccgccaccggacatctccgcgccctgcagacctcgttgtcggccatcaccgacgcccgcaacgccggccatctccgcctcctgcacccatcccgtcgctggccaccgctccgttgatttatttcctttcttccccaactccggcgggtcctctcccacggtgctacctgcggccacccatgggaggttccctgctgtcgcccatagctaacgccaccatagcacctctttttcctcaactctggtgactcctcctccggcgagacctcttccatgcggcggtgctacaccgcggacaagcgttgttgcgtcgatctattgggacaaggtgagcagtttcaatgggtgaattggttgctctcttgattccattagcaatacttcgcacaaccaaacatcaaaattggtattggaacccaattcaatctagaggctagctgattggtatttagctcaattcaatttcatgccttccaatacatacatccagacgaagcttaagatctcaatatcaccattattttcgttctttggtacggagtatatacttcgacaacaccttagtttatttttttttagctggagcgcatacgccgttaccgtgtcaggtgtgacggacgaaatgcccgcacgaatcgacagcatgcacgggtaagatgacagctagcaacagcgggtacacaatcatcacgagccatccgatgggagctttgcgccgcctttgtgagcggacggacaggatcgctctcccaaacccctcaaattacgtacggggtcacgcctcgacaaaccgtcgtccttcctccctaaaccacccgcaccaccccagagagagaggggcacgcggac

SEQ ID NO: 43 - DNA, 639 bp, Brachypodium distachyon
  source 1..639: mol_type = genomic DNA
atgggacccaccgccctccacgtcgtcatcgccgtcgcggccctcctcctcgccgccgccgcctccgccgcgtcgtcccaggagcctccggcgacgccgtgcgcggcggccatcgtggccttctcgccgtgcctggcgcacgtcgcggtggtggcgccgcccgccgtggccgcgcccgcgcccaccggcgcctgctgcgcggcgttcctgcgcgccgtctcggccggggacggcgaaggcggcggcggggagggatgcttctgccacctcgtccgcgacccgctcctcttcggcttccccgtcgacgtcgggcgcctcggcgcgctcctccccacctgcgcctccgccaacgcctccgcagccaccaccgtcgaggccgaggccctcttcgccgacaagtgccgagagctcaagtcactgcctgagatgcatttgtcacctccatcaccacctcctgcgccaaaactttctccagctgccgtcccaggaccagcgtcgtccccgaaggtggaggcggcgcaatcgacgacgtccacgccacgtgatcggtcaggatcggatggcttgtgtgccttccgggtttcgctcatggcgttggtcttcacagcagcagtcttgatcatgttgcagctctggtag

SEQ ID NO: 44 - protein, 212 aa, Brachypodium distachyon
MGPTALHVVIAVAALLLAAAASAASSQEPPATPCAAAIVAFSPCLAHVAVVAPPAVAAPAPTGACCAAFLRAVSAGDGEGGGGEGCFCHLVRDPLLFGFPVDVGRLGALLPTCASANASAATTVEAEALFADKCRELKSLPEMHLSPPSPPPAPKLSPAAVPGPASSPKVEAAQSTTSTPRDRSGSDGLCAFRVSLMALVFTAAVLIMLQLW

SEQ ID NO: 45 - DNA, 1000 bp, Brachypodium distachyon
  source 1..1000: mol_type = genomic DNA
acgcttgccctcgctctcgacacacatacttgtgcctgctccgtgtttcttgatcggctcctaatgttggctatggtgaagtgtcatgtgatgccgcgtggcaggcttataatctctgaaaactcgtccttactactggtgcagtttttcgtatgcgtagtctgacagtacaggtaactgctgattggtgacattcatgtaccgttgcaaacatggtaggaacacgtacaatgcagttttacaccctatcctttccgagacacaaaactgaaatgaccgggattttatttcactagagtatatagctgcgccattgcacggacgaacaaattcagctagtgatcataagagagttgcacacttgagattactggtgcatctttacatgccattcggtgtctgatcaatttgagtcaactggacgaacaaattgtacgcgcggcacatgactacataatcagcgacagactgacagtgcccctgccacgatcgaggaagcgaggaccaaagtggataccccttgacaccgactccggcgagcgctggacgggttgaacgcgcggtcttgcgggtacatctgcccgggggtgcccgttatgcccatcaccggcgccggcggcccgtacgtgtcaccccacgggtactcgtacggctcagccggcggcggtaggtaatactctggctccgacggcggcgggtagtagtagcccggcgtcggctcaggctccgacggcggtgggtagcaccctggcgtcggctcaggctccgacggcggcggatagtactctggcgtcggctcaggctcagacggaggcgggtagtactcgcgttccggcggcggcgggtggtcagtcggcagagctggtggaagcgggtgcggatccacgtcgcagaggcacatgaagcacttgcacttgtggtctgtttcgacccccaatgaccgggacacgaccatgcacatggacacgaggattgttatcttgagatgcaacattttgct

SEQ ID NO: 46 - DNA, 1772 bp, Oryza sativa
  source 1..1772: mol_type = genomic DNA
atgggccccaccgcgctccacctcatcgccgtcgccgtcgccgccgtcgtggccgcggcggcggcaccggcttcggcgtcggcgtcggcggccggggcgttctcggaggtgccgccggagacgccgtgcgcggcggccatcgtgtcggtggcgccgtgcctggcgcacgtcgcggtggtggcgccgcccgcgcggcccgcgcccgcgcccacggaggcctgctgcgcggccttcctccgcggcgtctcccccagcggcggcggcggggaggggtgcttctgccacctgctccgcgacccgctcctccttggcttccccgtcaacaccgcgaggctcggcgcgctcctccccacctgcgccgccgccaacgccaacgcctccgcggccgccgccgtcgaggccgccacgctcttcgccgacacgtgccgaggtcattaccgtgtgccccccttcttcctccttttacatgtccttgcttccatcggttttgcgtattcttgtgttcgtgatcaccgatcatgttggagaactactgaactagtatgtgcattcacgacggttggtgatagatgcaaatagttggcgagccgcaagctctagatactactgagagcaaatagattgaaccagataaatttattcgagaaatgaaatgaaatttatttgtgaacagataaaatttagtttggttagtagttactgggaattgtattactgtgtgaaactattgaattggaaaacagtgtttgcctatttttgtgtctatccataaacgtcaaccttacttcataaattctcgttttatccaaactactaaaaagttgcagagatacaaatgtaggacaaattgatactacggtcatcaaggaaatttgcaaattgcaatgcatgacacacattgaaagccttgtttggcatcaatttcaccaaatttcttgagctttacaatatttcattctcagataatacgtcattgcttttctcttagagagttcttaaaactattcactgaacttgatttgttaattgacatcatgagttcttgattctttatcatctttgtttgtttcacccctctaaagcagacctcaagtcactgccagagatgcgtttcctacctgacccacctcccacgccaacaatttctccaggtaaacgttattcagcttgttcacttatcctgtgaccttttggccactgactccattcgtgagataatcctcatccgtatcatcctgtacttcgcctctgctcacatgttgttttgtctcaattttgtgttatgtgcagctataaaactatgatagtagaaatttgaacttagtaataccacgaaaagaatcacatacgtaattgtgcgatcatggtatactgttacttgcagcatctcttctgttgagttgtaagacttgtaactgttactaccatttccagcatggagtgtcctgctgatgctatactatatgtataatcttctcatctttcagttgtaacagtactttttagctcctgtatgaccatgatcatatcgtctatttcatcgtttaatcacagtcacggcaacgcctatgtggactcgctaatctgttgtgaatgcctgtgcagctgccgtcccaggatcaatgcccccgacgacggaggagcgttcgacgcccgtgcctgtgccgccacaggaccggtctggatcggagacctcaactcccagccggaatttccttgtggtgttgttagccctaacagcagcagcagcggcagatttgatccagctgtag

SEQ ID NO: 47 - DNA, 2000 bp, Oryza sativa
  source 1..2000: mol_type = genomic DNA
ttcttggaacactttgacaggttgttgtcagacaaaagatatgaggaagtcaaaaatgactttaagacaacacaaagtacaccatggccgaaagcagatttgacaatattaaggcacaacaagaatttatactccagcaatatttaaagtgtttcaggaacaagtgttgcaaacattgaactgtgacctatactggtgtacaaattaaaagttcgcggtaagcaccattagcatgttgtaaaattttctccaacagaaggtaaagtcaggtgcagctgcaagaagtttgaatttctcggaatcctatgttgccatgcattgaagatacttgatgttaacaatataaaagaaattcctaagcaatacatactacaaaggtggaccattgatgcaaaatctctacatataaagagcaactgcagtactcatgaagatcctaaaataaagttatcaacgcgcaggagagacttgtgcagaatgtttgttaaaatagcatctcgagctgcagaatctgatgaaacatacttaatggctgccaacaatgcacaaaagttagcagaagatgtggagaaatacttgagtataaggcctgatccagatttggataaatctggtttgtatcctactctgagttgaatagtacttatgctagtgactactatgttataatctaaatcagctatctcaaaaatattttatcataggcacagaggaaaatgttgacccgagtaaagcaagaggcataaaggttaaagagaaagccattcgtgggtcgaggagaccaattggtggttttgacaaggcaacacaacggagtaaaaagaaaaagagtgattcaaacacatcaaagtgtccagtacaagcagaggtggtaacaccatcactgccatacaccatgatgcaggtcagtagattcaagatatacacacacttggcaaaagagactcttattagtgctactgattattatttgccttatacagattcagggtcgttcagagattcccactaactacaatcacatgcaaatgccagactactatcatgttgaaggtgcttcattacttcagtcatctggttatttcagaacttctgaaaaaagtcaagggatgcagcatgaagagactgcacagacatttgatacatatccctatagcatgtttgcccatttaaattaaaatccccaccttttttcatattaattagcacctttaccaatttccaatcacattacaacttataaggttggatcttaatctgaattcccatatcgattcaagctgtcaaatcatactagtactttctcttgtattagcatcaaatatatggttcccaccaaacatgttgtttggctcatataaaaaaaatcctaataatttagaagcatcaaaatacaagaggtgtttgcaaaaatgaacttaccaaataaaactaccttaatgcagctgtatggtgaaaaaagtttcgatcttcaattcttcacttcttcaccatagcaaacagtagttgccagcagcaagggagttccacatttggtactgaccattaaactacaccaagccgacgagtatgcggagtgcagaccacgccaagccggcgagcccggccgccgacaacgcaagagcaaccatggttgcgtgcggccaagaggaagatgaggagagcagcagatgagcagaacttcagatgaactctctgatttggggacaagagataaggatgcagtggttttccaagttgtgcagcgcggagtgaaaagtactagtacccggtttgttttgacggagtagtgacggcgtcatattctgcacggcagatagaagccgaccccaccaccaaaaagaatcgacaaaccgatgccctccacgtggtacggaacacggatggaaacacaacaacggaacagtgaagccgcagcccctcctccaaaacccctccgtccccacagacacacggggcccacctacagaggcagagagcgcgcgccgcggcgagagcac

SEQ ID NO: 48 - DNA, 666 bp, Oryza sativa
  source 1..666: mol_type = genomic DNA
atgggccccaccgcgctccacctcatcgccgtcgccgtcgccgccgtcgtggccgcggcggcggcaccggcttcggcgtcggcgtcggcggccggggcgttctcggaggtgccgccggagacgccgtgcgcggcggccatcgtgtcggtggcgccgtgcctggcgcacgtcgcggtggtggcgccgcccgcgcggcccgcgcccgcgcccacggaggcctgctgcgcggccttcctccgcggcgtctcccccagcggcggcggcggggaggggtgcttctgccacctgctccgcgacccgctcctccttggcttccccgtcaacaccgcgaggctcggcgcgctcctccccacctgcgccgccgccaacgccaacgcctccgcggccgccgccgtcgaggccgccacgctcttcgccgacacgtgccgagcagacctcaagtcactgccagagatgcgtttcctacctgacccacctcccacgccaacaatttctccagctgccgtcccaggatcaatgcccccgacgacggaggagcgttcgacgcccgtgcctgtgccgccacaggaccggtctggatcggagacctcaactcccagccggaatttccttgtggtgttgttagccctaacagcagcagcagcggcagatttgatccagctgtag

SEQ ID NO: 49 - protein, 221 aa, Oryza sativa
MGPTALHLIAVAVAAVVAAAAAPASASASAAGAFSEVPPETPCAAAIVSVAPCLAHVAVVAPPARPAPAPTEACCAAFLRGVSPSGGGGEGCFCHLLRDPLLLGFPVNTARLGALLPTCAAANANASAAAAVEAATLFADTCRADLKSLPEMRFLPDPPPTPTISPAAVPGSMPPTTEERSTPVPVPPQDRSGSETSTPSRNFLVVLLALTAAAAADLIQL

SEQ ID NO: 50 - DNA, 1000 bp, Oryza sativa
  source 1..1000: mol_type = genomic DNA
ttctgatatgtcattgcccatgcccagtactcacgctcctgacgaacttatgcctgttgcgtctttcctctgtcggtcggctttggactcaaatcaggtggtgccgtgtgttgtacagatgcaggggaggatgatctctgagaaattttgtccggaatggtgatgttttctcttttggtgtgctccgatagtccgatccatggtatgtcagtacaagtgtgctgcttctggctatgtcatgcaccattgacaacattcatagggtacggatagaatatggtttatattctcccatagtcttcatgtcactggaagccaaagaagagcatttctgcagtcacacggtcatgcagcatccgcactcaatcgatgtagtaatatagagtccagtcgcgcacaagtcaaacagttaaacaaaacccgtttggctcaccttgtcaaaatgttggccatgcctgacgtcttcttctacaacctggccagctgggccgtttggttcggctcctcgcacctgattcacatatccagcttaattcctttaggattatttattctattttttaaaaaaaaatacatcctgtcaagaaaaatattctagtatttgcttagaccacttgtcttaaatagtttgttaaatttatttatcacaacatttaatttatccctattaaaaagtagataatctacaaaaaaacagtccatccaatattttctaaacaaagccttaagttgaacgattagcgaatggctggtgcaacaaaatcaacgtagctgcttccccatttacgttccaagatggccagcctatgtgctaactgctactaccttcatcgccacaagtcaactggaagtaggcatcagggttaccttctgcagaggcaacccagtctagtcaagcccagcaacttgcacaacttgaccaagaaggcaagaacacacacaccctcgcttgcttaaattctagctgaattccattcgattctagtctcactctcatgta

SEQ ID NO: 51 - DNA, 2543 bp, Zea mays
  source 1..2543: mol_type = genomic DNA
atgcccatcctcaaaacccaacccaaccaacttgagcgcgcgactcaaggcgccacaccggcaccggcatgcaccatgggtcctaccacagctcgcctccttcccctcctgcccttcgtcgccgtgttcctcctcgcctcccccgtggacgccgactccgccctggacgcggccacgcggtgcgcggccacgatcgtgtccatctcgccgtgcctgccgcacgtggcggccgtggcgccaccgctggctagctgcccgcctgccccaaccgacgcctgctgcgtcgccttcctccgcgctgtatccccgttggcagcgggaggcggagaggagggctgcctgtgccacctgctccgcaacccgcttctccttggctttcccatcgacgccgcccgcctcgccgcgctcctccctgcctgcgctgcgggaaatgccttcgccgccgccaacgtggaggccgccaccctcttcgctgacgcctgccgtggtgagggaatcacgccccctcgtgcctttctcccctctcgcaccatgatcgaagaatcatgtgcgttgttgcgtcaggttcctgtacgcgttctcgtcgcaatgctgctgtgtttgcgcgcttttgttatgcaacaaccgcatatggtggactagctcaagttgtggatacaaatagcactgcctgcttgcttgctgcctcctagacgcgccattatcgttcaatgggtagtcatcaacttaatcagtcgtatattgttctttcaaatttttagcgactctgtttgttctgacattaattgctgcaatagtagtatttctgctatttagcactggtgcgtagcatttagaattccttgcgttagtaagattgtatgcgatgtatagtatagggctaattattattggccaggtaagttttagcccagatctagcctatattttttagccccaactcgtaactaggcttagctgattcaagtagacccttagcaagactcgctcatttgtcgtgttgcttatcaaccgttattttgccttgaagtggaaatacatgctattgaagtgtataagtggattatctaatttctgtcgttctcttattagtttaatcttttgacttaaattgattagcacgtccactctaacatggtattgaagccagaggtctcgagttcgaatcctgacaaaagcgacagagactttttttatgtctctatctatttattttcacgtttgtgactttctctctggctgtacgtgagtggaggtgttgaagtgtataagtcgattgtctatcttttgctaatagcttaagcttttgggtgcaactggttagtgcgtcccctctaacacatgccaacatgtttcatttttgtaatagaagggaattgtacttttgcacacttttctttcataatttaacgatacaccattagtttggcatgtgataaacaaaatgttagtttcttaggaaccatgattggtcatctacttggggcgtatctaatgccataggcttcccacactatgcagggtctgggaaagagtatctttaagtgtaagccttacccgcataatatgcagaggttggagcactggccatctactaatcttaatcaattttccatgttttgatttaagagtcatttttgttctcaaacaaatttattgtaacagtgtgatagttctgtcacatgtcatatcctgatggtttagcaggaaacaaaagttacctgttgctggtaaatttgtctcccttactataagttcacatatttttgttatatgttagattagaaggtgccatctttcttattgacatgcagatcgcgcatgtactgcttgtggtgtttatcctaatcatatgacgaaatgagataagagtataatattgcacaatccattaactttccacttatcattttgttgtttcatttcctgagcatagatctaaaggtgctgccgggattgcatttcatgccgcaatcaacaaccgggccagaaatttcaccaggtaaacatgcatatttcttcagtgtaccactccattttttttaaaaaaagggttcacactgctcgtctgtccaagcaaacttagtttgttgtgcaattacagtttgtgtttcgtgagatcggacatgttcataatgtatcgtaactacatgacacatcatgcatgcgatgcacatttcatagttcataacggtttagtactttgtgctattgtttatgtcaactactcttactgtgttgtaatgaagagtgctttgtaattttgttgccatgtatggactattgtgtacttaatttgtggtaatgagcacttgtacttgtctatgtcatggtttgccttcctatgaaggttatataaattaatctgttgatacatatgtcttggcgcagctgctgtccctgaattgatgtccaagccaaaggaggcggttccacttccaccagctggatcaatggttcgttcaggcactgaggtcaccagttcccgtggtattcctactgcagcattgatcctcgcggcagcaggagctgttatcacatag

SEQ ID NO: 52 - DNA, 2001 bp, Zea mays
  source 1..2001: mol_type = genomic DNA
caacagaagcattgaagcacgaagtgatatgaagactagccaaaggccatctaccgaacgtccaatctgttgcagaccaaataaatttaaaacctaacaagatatgggcagatcgcgtgctcgatctcaaaggcaaaactgtatgctgacttctaaagcataaagttgatgatataatgctgatctctaaggcattcgacaaaaggaaaggacaatttctgaaaaacagcgtgagatgaagaccttcgagtggattattttcagtaatccactcaaagctcgggggctacacccaatgaatcatcaaccccaagagacagaatgctgatttctaaagcataagatgaaactaagacaaggctgacttctaaaagcacaagtggaagataaaagtgatttctgagaagacttgaaatgaagaccttcaagtggattatttacaaaaaaccactcgaagctcgggggctacacccattgggtgcaccaatgaaatttgaattctaaagagcaaaatgctgaccaccaaagcataaactcgagatagagcaccgagttctgaggaataaagcgaaggccgacgaataatggcagaagaagacaatagaagatcgtttttaccgggtcactcagagttcagaagcaattgcttgacagacttattttcaaggcattccttatcaaaataaaaaactgcaagctggacctaggatctttgggccggttatttcaatatcaacccaaagatcgggggcttgtgggggacggatatcccccgggtccaccggaaggataaaagacctcacgaaaggcccgtggggccaataaatcgcaaggttatcccttcgtgggcctagagaggaacgtctagtgaagtggattgacacaaggccggattgacgcaagcccagacggcctgatgaatttaagcaatgatcgcaacagtgattcgacctgcccgtgcagtgccctcgtacatcggaactgaataagggataggtcggcggaattataggaagataggctcagttggttcactattacttaggcacacattgttatcatatccacatgtaacgccccacggtcgagtatataaggcctagggggcaccccctcgaaagcatctcattacttagccatccatccggttctctaacatcttcgacactagagagccttcttgtaatccaccacataaagtactcacgctaggacgtagggtgttacacatctcaaagcggcccgaacctgtacaaaattgtccactgtctctcgtgcatcaagcgcgaactatcgagctacagtcggtaacaccgtcctactccaaaaatcacctcgaggagcaaccccgggtgcgcggtcggacccaaaacaccataacacttagaatttataagacctatttttggtagttttatagaggagaataataaaaatggatagcattaaaacatgactatcaaatagatagatactacctccgttcttgaatatttgtcgctcactagttcacttttgaactaaaacgtgacaaataaaaaagaatgaagggagtacgatccagaaacaaagaagaatctatatgaaaacaaggtatcaacattgcagtaatctgattagtcctatgtaaaccgccttttagatgatgcacaaaatctgtctctagaaaacctatttctttatcgaggccatccttcatggagattttggaatgatgtatacatcaacacgtggtttcgctaaaatgcccatcaggccataggacttaccaaaatgttctactaatatgaatatattgctataatactcgtaccttttcaatttcataatatcatatcttatgttttgtaatcttcatattgaccaaaatacccttcttgcaaaagaacttactcttcctgatgtcattttcttgctaaaaaattaacacatattttgaacgaagccacataatacgtttattccaaatatctcctcgctgtctcctggctcaattttccc

SEQ ID NO: 53 - DNA, 705 bp, Zea mays
  source 1..705: mol_type = genomic DNA
atgcccatcctcaaaacccaacccaaccaacttgagcgcgcgactcaaggcgccacaccggcaccggcatgcaccatgggtcctaccacagctcgcctccttcccctcctgcccttcgtcgccgtgttcctcctcgcctcccccgtggacgccgactccgccctggacgcggccacgcggtgcgcggccacgatcgtgtccatctcgccgtgcctgccgcacgtggcggccgtggcgccaccgctggctagctgcccgcctgccccaaccgacgcctgctgcgtcgccttcctccgcgctgtatccccgttggcagcgggaggcggagaggagggctgcctgtgccacctgctccgcaacccgcttctccttggctttcccatcgacgccgcccgcctcgccgcgctcctccctgcctgcgctgcgggaaatgccttcgccgccgccaacgtggaggccgccaccctcttcgctgacgcctgccgtgatctaaaggtgctgccgggattgcatttcatgccgcaatcaacaaccgggccagaaatttcaccagctgctgtccctgaattgatgtccaagccaaaggaggcggttccacttccaccagctggatcaatggttcgttcaggcactgaggtcaccagttcccgtggtattcctactgcagcattgatcctcgcggcagcaggagctgttatcacatag

SEQ ID NO: 54 - protein, 234 aa, Zea mays
MPILKTQPNQLERATQGATPAPACTMGPTTARLLPLLPFVAVFLLASPVDADSALDAATRCAATIVSISPCLPHVAAVAPPLASCPPAPTDACCVAFLRAVSPLAAGGGEEGCLCHLLRNPLLLGFPIDAARLAALLPACAAGNAFAAANVEAATLFADACRDLKVLPGLHFMPQSTTGPEISPAAVPELMSKPKEAVPLPPAGSMVRSGTEVTSSRGIPTAALILAAAGAVIT

SEQ ID NO: 55 - DNA, 1000 bp, Zea mays
  source 1..1000: mol_type = genomic DNA
ttcttgtgatgtttaagtgccacttgatactctcgtcggcctcgctcgatcttgatctgcgtttgtcgtgcctagctttgagcctttgacgattcagatgatctgcatttgccatgcctagcactttgcggtctccagtgttgtacgtggcagagttgtgtcacttgacacttcaatggaatctagtgatcagctgataccttcagttgtttctcattcagaagcaggtcaaagatctactcagtcttgatgagatcgaagttgaaacccgccgatatatcaggagttgtggttcagggccaagagacagattgtgtgccccggcccccgggacaagtcgacaactgcgaacttggtttcatcagacgagtcgtcactttcctatcagacatgcatatataagatatcgattttttatacctttccctgataagcctcatgatgttgttgttcgaaatccgatgcccgtgccagcaattcgtcgatggactcaaagtttgtcatggtaactctgtcagggtcttagaccgaggggatatcatgcggtactctgggcagcagatgatgaactccaccttcatgccttcaggtgcgctgatctgcattggctccactccactggtcgagtatagagcaacttctctccttaatgtgacctgaacgtgggccctggtacatggtagttgtcggccaaaatagatctggccactcatgcaatgattccctccactaccggaatcgccggctttgccgagtgtcgcactcggcaaagagggctcggcatacagttcatcggcaaagccgtctttgccgagtactttttctcgggcactcggcaaagtggtttgccgagtgccagagagcactcggcaaagaaaagcagccgttacggcgacgggtgacggagacggagtctttgccgagtgtcccaggcgacactcggcaaaggagttacctttgccgagtgtcggcctgacagcactcggcaaagaatccgcta

SEQ ID NO: 56 - DNA, 22 bp, Triticum aestivum
  source 1..22: mol_type = genomic DNA
cgccaccagcagcagcccgcgg

SEQ ID NO: 57 - DNA, 23 bp, Triticum aestivum
  source 1..23: mol_type = genomic DNA
gggcggggggctgctgacgacgg

SEQ ID NO: 58 - DNA, 23 bp, Triticum aestivum
  source 1..23: mol_type = genomic DNA
gtcgtccccgccgccgtcccagg

SEQ ID NO: 59 - DNA, 23 bp, Triticum aestivum
  source 1..23: mol_type = genomic DNA
gacgaagaagaaggccgccttgg

SEQ ID NO: 60 - DNA, 23 bp, Triticum aestivum
  source 1..23: mol_type = genomic DNA
gcccacggcgccgtccaaggcgg

SEQ ID NO: 61 - DNA, 23 bp, Triticum aestivum
  source 1..23: mol_type = genomic DNA
ggccgtggcgacgaagaagaagg

SEQ ID NO: 62 - DNA, 23 bp, Triticum aestivum
  source 1..23: mol_type = genomic DNA
gtagaggccgagcatggccgtgg

SEQ ID NO: 63 - DNA, 23 bp, Triticum aestivum
  source 1..23: mol_type = genomic DNA
ggccttcttcttcgtcgccacgg

SEQ ID NO: 64 - DNA, 23 bp, Triticum aestivum
  source 1..23: mol_type = genomic DNA
gatgatgtagaggccgagcatgg

SEQ ID NO: 65 - DNA, 23 bp, Triticum aestivum
  source 1..23: mol_type = genomic DNA
gagatcccgcgggctgctgctgg

SEQ ID NO: 66 - DNA, 23 bp, Triticum aestivum
  source 1..23: mol_type = genomic DNA
gctgctggcggcgctgctgccgg

SEQ ID NO: 67 - DNA, 23 bp, Triticum aestivum
  source 1..23: mol_type = genomic DNA
gcacggcgagaaggacacgatgg

SEQ ID NO: 68 - DNA, 23 bp, Triticum aestivum
  source 1..23: mol_type = genomic DNA
gcagcaggcgctggtgggcgcgg

SEQ ID NO: 69 - DNA, 23 bp, Triticum aestivum
  source 1..23: mol_type = genomic DNA
gccgcgcagcaggcgctggtggg

SEQ ID NO: 70 - DNA, 23 bp, Triticum aestivum
  source 1..23: mol_type = genomic DNA
gaacgccgcgcagcaggcgctgg

SEQ ID NO: 71 - DNA, 23 bp, Triticum aestivum
  source 1..23: mol_type = genomic DNA
gcgcaggaacgccgcgcagcagg

SEQ ID NO: 72 - DNA, 23 bp, Triticum aestivum
  source 1..23: mol_type = genomic DNA
gcccaccagcgcctgctgcgcgg

SEQ ID NO: 73 - DNA, 23 bp, Triticum aestivum
  source 1..23: mol_type = genomic DNA
gccgccttcgccgtccccggagg

SEQ ID NO: 74 - DNA, 23 bp, Triticum aestivum
  source 1..23: mol_type = genomic DNA
ggggacggcgaaggcggcggagg

SEQ ID NO: 75 - DNA, 23 bp, Triticum aestivum
  source 1..23: mol_type = genomic DNA
gggacggcgaaggcggcggaggg

SEQ ID NO: 76 - DNA, 23 bp, Triticum aestivum
  source 1..23: mol_type = genomic DNA
ggacggcgaaggcggcggagggg

SEQ ID NO: 77 - DNA, 23 bp, Triticum aestivum
  source 1..23: mol_type = genomic DNA
ggcgaaggcggcggaggggaggg

SEQ ID NO: 78 - DNA, 23 bp, Triticum aestivum
  source 1..23: mol_type = genomic DNA
gccgaggcgcgcggcgtcgacgg

SEQ ID NO: 79 - DNA, 23 bp, Triticum aestivum
  source 1..23: mol_type = genomic DNA
gttttcgcggaggcgcaggtggg

SEQ ID NO: 80 - DNA, 23 bp, Triticum aestivum
  source 1..23: mol_type = genomic DNA
ggttttcgcggaggcgcaggtgg

SEQ ID NO: 81 - DNA, 23 bp, Triticum aestivum
  source 1..23: mol_type = genomic DNA
ggaggttttcgcggaggcgcagg

SEQ ID NO: 82 - DNA, 19 bp, synthetic construct
  misc_feature 1..19: note = DNA version of guideRNA to target DNA target
  source 1..19: mol_type = other DNA
cgccaccagcagcagcccg

SEQ ID NO: 83 - DNA, 20 bp, synthetic construct
  misc_feature 1..20: note = DNA version of guideRNA to target DNA target
  source 1..20: mol_type = other DNA
gggcggggggctgctgacga

SEQ ID NO: 84 - DNA, 20 bp, synthetic construct
  misc_feature 1..20: note = DNA version of guideRNA to target DNA target
  source 1..20: mol_type = other DNA
gtcgtccccgccgccgtccc

SEQ ID NO: 85 - DNA, 20 bp, synthetic construct
  misc_feature 1..20: note = DNA version of guideRNA to target DNA target
  source 1..20: mol_type = other DNA
gacgaagaagaaggccgcct

SEQ ID NO: 86 - DNA, 20 bp, synthetic construct
  misc_feature 1..20: note = DNA version of guideRNA to target DNA target
  source 1..20: mol_type = other DNA
gcccacggcgccgtccaagg

SEQ ID NO: 87 - DNA, 20 bp, synthetic construct
  misc_feature 1..20: note = DNA version of guideRNA to target DNA target
  source 1..20: mol_type = other DNA
ggccgtggcgacgaagaaga

SEQ ID NO: 88 - DNA, 20 bp, synthetic construct
  misc_feature 1..20: note = DNA version of guideRNA to target DNA target
  source 1..20: mol_type = other DNA
gtagaggccgagcatggccg

SEQ ID NO: 89 - DNA, 20 bp, synthetic construct
  misc_feature 1..20: note = DNA version of guideRNA to target DNA target
  source 1..20: mol_type = other DNA
ggccttcttcttcgtcgcca

SEQ ID NO: 90 - DNA, 20 bp, synthetic construct
  misc_feature 1..20: note = DNA version of guideRNA to target DNA target
  source 1..20: mol_type = other DNA
gatgatgtagaggccgagca

SEQ ID NO: 91 - DNA, 20 bp, synthetic construct
  misc_feature 1..20: note = DNA version of guideRNA to target DNA target
  source 1..20: mol_type = other DNA
gagatcccgcgggctgctgc

SEQ ID NO: 92 - DNA, 20 bp, synthetic construct
  misc_feature 1..20: note = DNA version of guideRNA to target DNA target
  source 1..20: mol_type = other DNA
gctgctggcggcgctgctgc

SEQ ID NO: 93 - DNA, 20 bp, synthetic construct
  misc_feature 1..20: note = DNA version of guideRNA to target DNA target
  source 1..20: mol_type = other DNA
gcacggcgagaaggacacga

SEQ ID NO: 94 - DNA, 20 bp, synthetic construct
  misc_feature 1..20: note = DNA version of guideRNA to target DNA target
  source 1..20: mol_type = other DNA
gcagcaggcgctggtgggcg

SEQ ID NO: 95 - DNA, 20 bp, synthetic construct
  misc_feature 1..20: note = DNA version of guideRNA to target DNA target
  source 1..20: mol_type = other DNA
gccgcgcagcaggcgctggt

SEQ ID NO: 96 - DNA, 20 bp, synthetic construct
  misc_feature 1..20: note = DNA version of guideRNA to target DNA target
  source 1..20: mol_type = other DNA
gaacgccgcgcagcaggcgc

SEQ ID NO: 97 - DNA, 20 bp, synthetic construct
  misc_feature 1..20: note = DNA version of guideRNA to target DNA target
  source 1..20: mol_type = other DNA
gcgcaggaacgccgcgcagc

SEQ ID NO: 98 - DNA, 20 bp, synthetic construct
  misc_feature 1..20: note = DNA version of guideRNA to target DNA target
  source 1..20: mol_type = other DNA
gcccaccagcgcctgctgcg

SEQ ID NO: 99 - DNA, 20 bp, synthetic construct
  misc_feature 1..20: note = DNA version of guideRNA to target DNA target
  source 1..20: mol_type = other DNA
gccgccttcgccgtccccgg

SEQ ID NO: 100 - DNA, 20 bp, synthetic construct
  misc_feature 1..20: note = DNA version of guideRNA to target DNA target
  source 1..20: mol_type = other DNA
ggggacggcgaaggcggcgg

SEQ ID NO: 101 - DNA, 20 bp, synthetic construct
  misc_feature 1..20: note = DNA version of guideRNA to target DNA target
  source 1..20: mol_type = other DNA
gggacggcgaaggcggcgga

SEQ ID NO: 102 - DNA, 20 bp, synthetic construct
  misc_feature 1..20: note = DNA version of guideRNA to target DNA target
  source 1..20: mol_type = other DNA
ggacggcgaaggcggcggag

SEQ ID NO: 103 - DNA, 20 bp, synthetic construct
  misc_feature 1..20: note = DNA version of guideRNA to target DNA target
  source 1..20: mol_type = other DNA
ggcgaaggcggcggagggga

SEQ ID NO: 104 - DNA, 20 bp, synthetic construct
  misc_feature 1..20: note = DNA version of guideRNA to target DNA target
  source 1..20: mol_type = other DNA
gccgaggcgcgcggcgtcga

SEQ ID NO: 105 - DNA, 20 bp, synthetic construct
  misc_feature 1..20: note = DNA version of guideRNA to target DNA target
  source 1..20: mol_type = other DNA
gttttcgcggaggcgcaggt

SEQ ID NO: 106 - DNA, 20 bp, synthetic construct
  misc_feature 1..20: note = DNA version of guideRNA to target DNA target
  source 1..20: mol_type = other DNA
ggttttcgcggaggcgcagg

SEQ ID NO: 107 - DNA, 20 bp, synthetic construct
  misc_feature 1..20: note = DNA version of guideRNA to target DNA target
  source 1..20: mol_type = other DNA
ggaggttttcgcggaggcgc

SEQ ID NO: 108 - DNA, 20 bp, Triticum aestivum
  source 1..20: mol_type = genomic DNA
cgccaccagcagcagcaccg

SEQ ID NO: 109 - DNA, 20 bp, Triticum aestivum
  source 1..20: mol_type = genomic DNA
cgccaccagcagcagctccg

SEQ ID NO: 110 - DNA, 20 bp, Triticum aestivum
  source 1..20: mol_type = genomic DNA
cgccaccagcagcagccccg

SEQ ID NO: 111 - DNA, 16 bp, Triticum aestivum
  source 1..16: mol_type = genomic DNA
cgccaccagcagcccg

SEQ ID NO: 112 - DNA, 56 bp, synthetic construct
  misc_feature 1..56: note = amplicon
  source 1..56: mol_type = other DNA
ctcggatcctccgtgagatcaaattactcygcctattgagacatcctgatatagtt

SEQ ID NO: 113 - DNA, 65 bp, synthetic construct
  misc_feature 1..65: note = amplicon
  source 1..65: mol_type = other DNA
cagtcatggaaaaggagcctgagaagggacgcggcgatgcgcrgttccttggcaatcgccttctt

SEQ ID NO: 114 - DNA, 65 bp, synthetic construct
  misc_feature 1..65: note = amplicon
  source 1..65: mol_type = other DNA
catccatctttgacttgttcttttatttacycttcttcatttaacttgctcaccgcgaagacttg

SEQ ID NO: 115 - DNA, 54 bp, synthetic construct
  misc_feature 1..54: note = amplicon
  source 1..54: mol_type = other DNA
gctgtagtgatttcactcctatcgcyaagcaaattgagtcagacaagtcgacta

SEQ ID NO: 116 - DNA, 65 bp, synthetic construct
  misc_feature 1..65: note = amplicon
  source 1..65: mol_type = other DNA
ctgcagccgacctccagctatttccacgacatcgtgccggggaartgtgcccagccattgggatg

SEQ ID NO: 117 - DNA, 52 bp, synthetic construct
  misc_feature 1..52: note = amplicon
  source 1..52: mol_type = other DNA
ctgatccactcaccagtatgaaatkataaacacagcacaggtaacaagagcc

SEQ ID NO: 118 - DNA, 60 bp, synthetic construct
  misc_feature 1..60: note = amplicon
  source 1..60: mol_type = other DNA
agtttgaattgcttaccacatgtcacrtggttgatctcatctcttacatcctttttgtag

SEQ ID NO: 119 - DNA, 51 bp, synthetic construct
  misc_feature 1..51: note = amplicon
  source 1..51: mol_type = other DNA
cttcttgtatagaggtgatcgcgttyatacagtcacacgccacacatactc

SEQ ID NO: 120 - DNA, 61 bp, synthetic construct
  misc_feature 1..61: note = amplicon
  source 1..61: mol_type = other DNA
ctcccggcttctggcactcraggtggcattcttctggcctaggatccccttgtggtccagc

SEQ ID NO: 121 - DNA, 51 bp, synthetic construct
  misc_feature 1..51: note = amplicon
  source 1..51: mol_type = other DNA
caagccacatagccaagggtgattamaatttcacacgtgttgctcaaccat

SEQ ID NO: 122 - DNA, 54 bp, synthetic construct
  misc_feature 1..54: note = amplicon
  source 1..54: mol_type = other DNA
caggactccctcataggggcttcrcgaaaatttaaaattgtagacttctatggc

SEQ ID NO: 123 - DNA, 54 bp, synthetic construct
  misc_feature 1..54: note = amplicon
  source 1..54: mol_type = other DNA
catgctcctctggatgcggaagagcatrggctaccctctggtggtcttcatgcg

SEQ ID NO: 124 - DNA, 54 bp, synthetic construct
  misc_feature 1..54: note = amplicon
  source 1..54: mol_type = other DNA
agcaccaaggcaaacaccaaccaatcraggaaactactgattgcggattaaaag

SEQ ID NO: 125 - DNA, 61 bp, synthetic construct
  misc_feature 1..61: note = amplicon
  source 1..61: mol_type = other DNA
gtggaaataaggcaagtgattcatcacatatgttgtygatccatggacagagtagtttgtg

SEQ ID NO: 126 - DNA, 57 bp, synthetic construct
  misc_feature 1..57: note = amplicon
  source 1..57: mol_type = other DNA
cctcatgagtataaatgacacctcaayttgctaatttgacaagaacaatgcctataa

SEQ ID NO: 127 - DNA, 86 bp, synthetic construct
  misc_feature 1..86: note = amplicon
  source 1..86: mol_type = other DNA
tatgacctcaaatttgttccagtagatattrtatccacagacagaaaggggaagtaaatttgaaatgtaattgcggtgccaaactg

SEQ ID NO: 128 - DNA, 54 bp, synthetic construct
  misc_feature 1..54: note = amplicon
  source 1..54: mol_type = other DNA
ggaattaggtcaaatggagctgtgtrggacccatgagcttaggagggcgtgtcg

SEQ ID NO: 129 - DNA, 58 bp, synthetic construct
  misc_feature 1..58: note = amplicon
  source 1..58: mol_type = other DNA
atgacgattctcatttgtgcagataaatrttaaggttaaggttgtttctggatctcct

SEQ ID NO: 130 - DNA, 66 bp, synthetic construct
  misc_feature 1..66: note = amplicon
  source 1..66: mol_type = other DNA
gctgcgagtagcaagttaagatcatatgracaagtaaaaataaggcagaaagatggggataacatg

SEQ ID NO: 131 - DNA, 69 bp, synthetic construct
  misc_feature 1..69: note = amplicon
  source 1..69: mol_type = other DNA
ccccgttccattttataatgcgtatgattttttctctcakattccgaaatgtagtgcgtatatgcagca

SEQ ID NO: 132 - DNA, 19 bp, synthetic construct
  misc_feature 1..19: note = primer
  source 1..19: mol_type = other DNA
acgacgcctgtgtcggatg

SEQ ID NO: 133 - DNA, 22 bp, synthetic construct
  misc_feature 1..22: note = primer
  source 1..22: mol_type = other DNA
caccgtatgcaacaagaggtcc

SEQ ID NO: 134 - DNA, 19 bp, synthetic construct
  misc_feature 1..19: note = primer
  source 1..19: mol_type = other DNA
cagaaccagcgtccccgaa

SEQ ID NO: 135 - DNA, 23 bp, synthetic construct
  misc_feature 1..23: note = primer
  source 1..23: mol_type = other DNA
ttcaccgtatgcaacaagaggat

SEQ ID NO: 136 - DNA, 22 bp, synthetic construct
  misc_feature 1..22: note = primer
  source 1..22: mol_type = other DNA
gtcggatgatcggtcgggatct

SEQ ID NO: 137 - DNA, 21 bp, synthetic construct
  misc_feature 1..21: note = primer
  source 1..21: mol_type = other DNA
catcgtccagatgcagagagg

SEQ ID NO: 138 - DNA, 48 bp, synthetic construct
  misc_feature 1..48: note = primer
  source 1..48: mol_type = other DNA
gaaggtgaccaagttcatgctaactatatcaggatgtctcaataggca

SEQ ID NO: 139 - DNA, 46 bp, synthetic construct
  misc_feature 1..46: note = primer
  source 1..46: mol_type = other DNA
gaaggtcggagtcaacggattctatatcaggatgtctcaataggcg

SEQ ID NO: 140 - DNA, 25 bp, synthetic construct
  misc_feature 1..25: note = primer
  source 1..25: mol_type = other DNA
ctcggatcctccgtgagatcaaatt

SEQ ID NO: 141 - DNA, 44 bp, synthetic construct
  misc_feature 1..44: note = primer
  source 1..44: mol_type = other DNA
gaaggtgaccaagttcatgctaagaaggcgattgccaaggaact

SEQ ID NO: 142 - DNA, 43 bp, synthetic construct
  misc_feature 1..43: note = primer
  source 1..43: mol_type = other DNA
gaaggtcggagtcaacggattagaaggcgattgccaaggaacc

SEQ ID NO: 143 - DNA, 25 bp, synthetic construct
  misc_feature 1..25: note = primer
  source 1..25: mol_type = other DNA
cagtcatggaaaaggagcctgagaa

SEQ ID NO: 144 - DNA, 52 bp, synthetic construct
  misc_feature 1..52: note = primer
  source 1..52: mol_type = other DNA
gaaggtgaccaagttcatgctcatccatctttgacttgttcttttatttact

SEQ ID NO: 145 - DNA, 51 bp, synthetic construct
  misc_feature 1..51: note = primer
  source 1..51: mol_type = other DNA
gaaggtcggagtcaacggattatccatctttgacttgttcttttatttacc

SEQ ID NO: 146 - DNA, 25 bp, synthetic construct
  misc_feature 1..25: note = primer
  source 1..25: mol_type = other DNA
caagtcttcgcggtgagcaagttaa

SEQ ID NO: 147 - DNA, 44 bp, synthetic construct
  misc_feature 1..44: note = primer
  source 1..44: mol_type = other DNA
gaaggtgaccaagttcatgctgtagtgatttcactcctatcgct

SEQ ID NO: 148 - DNA, 44 bp, synthetic construct
  misc_feature 1..44: note = primer
  source 1..44: mol_type = other DNA
gaaggtcggagtcaacggattgtagtgatttcactcctatcgcc

SEQ ID NO: 149 - DNA, 28 bp, synthetic construct
  misc_feature 1..28: note = primer
  source 1..28: mol_type = other DNA
tagtcgacttgtctgactcaatttgctt

SEQ ID NO: 150 - DNA, 42 bp, synthetic construct
  misc_feature 1..42: note = primer
  source 1..42: mol_type = other DNA
gaaggtgaccaagttcatgctcatcccaatggctgggcacat

SEQ ID NO: 151 - DNA, 42 bp, synthetic construct
  misc_feature 1..42: note = primer
  source 1..42: mol_type = other DNA
gaaggtcggagtcaacggattcatcccaatggctgggcacac

SEQ ID NO: 152 - DNA, 22 bp, synthetic construct
  misc_feature 1..22: note = primer
  source 1..22: mol_type = other DNA
ctgcagccgacctccagctatt

SEQ ID NO: 153 - DNA, 44 bp, synthetic construct
  misc_feature 1..44: note = primer
  source 1..44: mol_type = other DNA
gaaggtgaccaagttcatgctgatccactcaccagtatgaaatg

SEQ ID NO: 154 - DNA, 48 bp, synthetic construct
  misc_feature 1..48: note = primer
  source 1..48: mol_type = other DNA
gaaggtcggagtcaacggattaactgatccactcaccagtatgaaatt

SEQ ID NO: 155 - DNA, 25 bp, synthetic construct
  misc_feature 1..25: note = primer
  source 1..25: mol_type = other DNA
ggctcttgttacctgtgctgtgttt

SEQ ID NO: 156 - DNA, 48 bp, synthetic construct
  misc_feature 1..48: note = primer
  source 1..48: mol_type = other DNA
gaaggtgaccaagttcatgctagtttgaattgcttaccacatgtcaca

SEQ ID NO: 157 - DNA, 47 bp, synthetic construct
  misc_feature 1..47: note = primer
  source 1..47: mol_type = other DNA
gaaggtcggagtcaacggattgtttgaattgcttaccacatgtcacg

SEQ ID NO: 158 - DNA, 30 bp, synthetic construct
  misc_feature 1..30: note = primer
  source 1..30: mol_type = other DNA
ctacaaaaaggatgtaagagatgagatcaa

SEQ ID NO: 159 - DNA, 45 bp, synthetic construct
  misc_feature 1..45: note = primer
  source 1..45: mol_type = other DNA
gaaggtgaccaagttcatgcttcttgtatagaggtgatcgcgttt

SEQ ID NO: 160 - DNA, 44 bp, synthetic construct
  misc_feature 1..44: note = primer
  source 1..44: mol_type = other DNA
gaaggtcggagtcaacggattcttgtatagaggtgatcgcgttc

SEQ ID NO: 161 - DNA, 25 bp, synthetic construct
  misc_feature 1..25: note = primer
  source 1..25: mol_type = other DNA
gagtatgtgtggcgtgtgactgtat

SEQ ID NO: 162 - DNA, 39 bp, synthetic construct
  misc_feature 1..39: note = primer
  source 1..39: mol_type = other DNA
gaaggtgaccaagttcatgctcccggcttctggcactca

SEQ ID NO: 163 - DNA, 39 bp, synthetic construct
  misc_feature 1..39: note = primer
  source 1..39: mol_type = other DNA
gaaggtcggagtcaacggattcccggcttctggcactcg

SEQ ID NO: 164 - DNA, 22 bp, synthetic construct
  misc_feature 1..22: note = primer
  source 1..22: mol_type = other DNA
gctggaccacaaggggatccta

SEQ ID NO: 165 - DNA, 45 bp, synthetic construct
  misc_feature 1..45: note = primer
  source 1..45: mol_type = other DNA
gaaggtgaccaagttcatgctggttgagcaacacgtgtgaaattg

SEQ ID NO: 166 - DNA, 47 bp, synthetic construct
  misc_feature 1..47: note = primer
  source 1..47: mol_type = other DNA
gaaggtcggagtcaacggattatggttgagcaacacgtgtgaaattt

SEQ ID NO: 167 - DNA, 25 bp, synthetic construct
  misc_feature 1..25: note = primer
  source 1..25: mol_type = other DNA
caagccacatagccaagggtgatta

SEQ ID NO: 168 - DNA, 52 bp, synthetic construct
  misc_feature 1..52: note = primer
  source 1..52: mol_type = other DNA
gaaggtgaccaagttcatgctgccatagaagtctacaattttaaattttcgt

SEQ ID NO: 169 - DNA, 51 bp, synthetic construct
  misc_feature 1..51: note = primer
  source 1..51: mol_type = other DNA
gaaggtcggagtcaacggattccatagaagtctacaattttaaattttcgc

SEQ ID NO: 170 - DNA, 22 bp, synthetic construct
  misc_feature 1..22: note = primer
  source 1..22: mol_type = other DNA
caggactccctcataggggctt

SEQ ID NO: 171 - DNA, 43 bp, synthetic construct
  misc_feature 1..43: note = primer
  source 1..43: mol_type = other DNA
gaaggtgaccaagttcatgctcctctggatgcggaagagcata

SEQ ID NO: 172 - DNA, 42 bp, synthetic construct
  misc_feature 1..42: note = primer
  source 1..42: mol_type = other DNA
gaaggtcggagtcaacggattctctggatgcggaagagcatg

SEQ ID NO: 173 - DNA, 23 bp, synthetic construct
  misc_feature 1..23: note = primer
  source 1..23: mol_type = other DNA
cgcatgaagaccaccagagggta

SEQ ID NO: 174 - DNA, 47 bp, synthetic construct
  misc_feature 1..47: note = primer
  source 1..47: mol_type = other DNA
gaaggtgaccaagttcatgcttttaatccgcaatcagtagtttcctt

SEQ ID NO: 175 - DNA, 46 bp, synthetic construct
  misc_feature 1..46: note = primer
  source 1..46: mol_type = other DNA
gaaggtcggagtcaacggattttaatccgcaatcagtagtttcctc

SEQ ID NO: 176 - DNA, 25 bp, synthetic construct
  misc_feature 1..25: note = primer
  source 1..25: mol_type = other DNA
agcaccaaggcaaacaccaaccaat

SEQ ID NO: 177 - DNA, 45 bp, synthetic construct
  misc_feature 1..45: note = primer
  source 1..45: mol_type = other DNA
aaggtgaccaagttcatgctcacaaactactctgtccatggatca

SEQ ID NO: 178 - DNA, 45 bp, synthetic construct
  misc_feature 1..45: note = primer
  source 1..45: mol_type = other DNA
gaaggtcggagtcaacggattacaaactactctgtccatggatcg

SEQ ID NO: 179 - DNA, 29 bp, synthetic construct
  misc_feature 1..29: note = primer
  source 1..29: mol_type = other DNA
gtggaaataaggcaagtgattcatcacat

SEQ ID NO: 180 - DNA, 48 bp, synthetic construct
  misc_feature 1..48: note = primer
  source 1..48: mol_type = other DNA
gaaggtgaccaagttcatgctcctcatgagtataaatgacacctcaat

SEQ ID NO: 181 - DNA, 47 bp, synthetic construct
  misc_feature 1..47: note = primer
  source 1..47: mol_type = other DNA
gaaggtcggagtcaacggattctcatgagtataaatgacacctcaac

SEQ ID NO: 182 - DNA, 30 bp, synthetic construct
  misc_feature 1..30: note = primer
  source 1..30: mol_type = other DNA
ttataggcattgttcttgtcaaattagcaa

SEQ ID NO: 183 - DNA, 51 bp, synthetic construct
  misc_feature 1..51: note = primer
  source 1..51: mol_type = other DNA
gaaggtgaccaagttcatgctatgacctcaaatttgttccagtagatatta

SEQ ID NO: 184 - DNA, 49 bp, synthetic construct
  misc_feature 1..49: note = primer
  source 1..49: mol_type = other DNA
gaaggtcggagtcaacggattgacctcaaatttgttccagtagatattg

SEQ ID NO: 185 - DNA, 29 bp, synthetic construct
  misc_feature 1..29: note = primer
  source 1..29: mol_type = other DNA
cagtttggcaccgcaattacatttcaaat

SEQ ID NO: 186 - DNA, 47 bp, synthetic construct
  misc_feature 1..47: note = primer
  source 1..47: mol_type = other DNA
gaaggtgaccaagttcatgctggaattaggtcaaatggagctgtgta

SEQ ID NO: 187 - DNA, 46 bp, synthetic construct
  misc_feature 1..46: note = primer
  source 1..46: mol_type = other DNA
gaaggtcggagtcaacggattgaattaggtcaaatggagctgtgtg

SEQ ID NO: 188 - DNA, 22 bp, synthetic construct
  misc_feature 1..22: note = primer
  source 1..22: mol_type = other DNA
cgacacgccctcctaagctcat

SEQ ID NO: 189 - DNA, 50 bp, synthetic construct
  misc_feature 1..50: note = primer
  source 1..50: mol_type = other DNA
gaaggtgaccaagttcatgctatgacgattctcatttgtgcagataaata

SEQ ID NO: 190 - DNA, 48 bp, synthetic construct
  misc_feature 1..48: note = primer
  source 1..48: mol_type = other DNA
gaaggtcggagtcaacggattgacgattctcatttgtgcagataaatg

SEQ ID NO: 191 - DNA, 29 bp, synthetic construct
  misc_feature 1..29: note = primer
  source 1..29: mol_type = other DNA
aggagatccagaaacaaccttaaccttaa

SEQ ID NO: 192 - DNA, 47 bp, synthetic construct
  misc_feature 1..47: note = primer
  source 1..47: mol_type = other DNA
gaaggtgaccaagttcatgctgcgagtagcaagttaagatcatatgg

SEQ ID NO: 193 - DNA, 47 bp, synthetic construct
  misc_feature 1..47: note = primer
  source 1..47: mol_type = other DNA
gaaggtcggagtcaacggattgcgagtagcaagttaagatcatatga

SEQ ID NO: 194 - DNA, 29 bp, synthetic construct
  misc_feature 1..29: note = primer
  source 1..29: mol_type = other DNA
catgttatccccatctttctgccttattt

SEQ ID NO: 195 - DNA, 47 bp, synthetic construct
  misc_feature 1..47: note = primer
  source 1..47: mol_type = other DNA
gaaggtgaccaagttcatgctgcatatacgcactacatttcggaata

SEQ ID NO: 196 - DNA, 47 bp, synthetic construct
  misc_feature 1..47: note = primer
  source 1..47: mol_type = other DNA
gaaggtcggagtcaacggattgcatatacgcactacatttcggaatc

SEQ ID NO: 197 - DNA, 29 bp, synthetic construct
  misc_feature 1..29: note = primer
  source 1..29: mol_type = other DNA
ccccgttccattttataatgcgtatgatt

SEQ ID NO: 198 - DNA, 4472 bp, synthetic construct
  misc_feature 1..4472: note = synthesized
  source 1..4472: mol_type = other DNA
ctcgagtgtcatgatttcagatctaaaccctttatgtttttatcaatatatctgagttcctaatcctatatggtagttatgtgcactttttattgttctgaaccgtagaccccaaggtagcaacatgaaaggggggattaaagggttgaaaacattgatcatgaaagagtccccctctgcctattacatccactgttttgcacatcaacttcaattggttcttacagccgtggcaaaagaaaatgaaccatgttcgtggttctttgatcatgtttcttaattgcttaatattcttggagtttcttgcaagtgacatgacatgctttgagatgttagagctcaaaaggttttggaagcacttgaaatgggtgagattgaaagtggaagtgggctaaatcaagagatgggactagctagacccggcgatactagttggggttctcattttaagaccattatgcacattgttagcatgtatccacaatccttgaagtacttgatgctattggaaaagatccttcacaaaaaggcgagtggacaagaatatgttgagttactcatgcttttgttttcaatcttcgtttgatgctagttattcgtggctatacaaatgagttgtccaaatctttgcaaaagagagatcaagatattgttaatgcaatgacacttgttagtttggcaaatagtagaatgcaacacatgaggtctcatggttgggaagaatttcttgcaaagatgaccttatttggcaacaaaaatgacactgaagttcatttgtggtaggatacttataagcctcatggaagatcacgtcggtattatgaagtacaaacaaatgatgatcattatagaagagaaatgtatcttggtgtcattgattaaaccattcaagagcttgacaatcggtttgatgaggtttacatggagttacttatttgcatgttggctttgaatcccctcaattcatttgcttctgacgatgcaatcaaggtaatgagacttgtcgcattctatcccatggacatatcaagtacatatttgataaggctataatttcaacttgctaattttgttgatgatacgagacaatacgataggtttagaaatgcaagtaatattggtgagctctctattatgcttgttgcaacaatgaaacatgttctttatgatttggtctacttactcatcaaattgatattgattttaccgatggtgactgcgagtgttgaaagagtattttggatcatgaatatagcgaaaagtaagttaaggactagtatgagtgatgaccgcttgaatgattgcttggtgatatttattgagcgggatgtgttcatgaaagcatgtgaagatgacatagttgatgctttcatggcaatgcaaaacgtagagttacctactgttatgattttctacttgtgcttctttcatgtaagactattcgtattgttggagatttgaattgaactatccttgttattctttttgccatgttttgtttgacacacttggattaggtacagaaaaaaatagtgtgcacgcccttcgttttattcctgggttgtcattaggccgtaccaggagcgatcgtgtcctgttgattattaagaccacagtgatctggctcagacggtagtaaactggatcgtgttaatttaggttaagttataatgaaacttcttatcccgcaaaaaagttaatggaacccctttccttcgaaaaaaagataacggacgaatgcccgcagaaaaaccgagtgttgatgacagctagcgacaggcactggatcatcacgagccgtcggatgtgcccaactttgcgccgcctttgttaacggacgggcatgatcgcttttgcaaacccctcagcattacacactcacaccctaaaaccaccaggcatcatcacctcaccacccatcccgccccgatacgagagagagagtggggacgggtgtgctgagagcctgagacatggcccccaccgccctcctcatcgtcgtcctcgccgtcgctgccctccacgcccccgccgcctccgccgcgttgtcccaggagccaccagcgacgccgtgcgcggcagccatcgtgtccttctcgccgtgcctggcgcacgtcgcggtggtggcgccgcccgccctgccctcgcccgcgcccaccagcgcctgctgcgcggcgttcctgcgcgccgtttcctccggggacgggatgcttctgccacctgctccgcaacccgctcctcctcggcttccccgtcgacgccgcgcgcctcggcactctcctccccacctgcgcctccgcgaaaacctccgccgccacggccgccgaggccgaggccctcttcgccgacaagtgccgaggtgagaaatctgtcccttcgtgctccctatttatgctcgtgcaatatgtatgcttcgatcaattctcgtgcgccatatgcgcgtgctgtcgcgttcctgttgttgatcgccgatcgaagcaaattttactctgcaaagtcctaactactgttgttctcatgatccttgtacaactactttcgtaatggttggttgtaatgcggattactgacgagctctgaatgctactagggatattagcacttgcattactgaactagtggaatgggggagaaacaccgcgggatttttcttttgttattaaggagaagatactgagtggcccagacttacttctgcgtttttgctgcgatttgtcatgattaccgttgatttaagcagtttgttgggttgcttgtttgatagtagtagtaatttcacaaaatattggcgatatttataaatagctaagggcttcgttacttggtggtttctctcaaagagttcacaaaagtcaattattaaattcaattaagggggcaagtattagtagtactggctaactccatttgccatttgccaattgaaacagagctcaagtcactgcctgagatgcattttacacctccatcgccacctcccgcaccaaaactttctccaggtaaatgttctgctgcttgtctaatgattccatagcttgttaaaaaaaatgattccataaatctgtgcccagtaatgctatttcggatttcggttgaatgaaccaattggcatttgggcagacatgtcatatgtcctctcctaccaaatgaaacttgaacttgttttatcttgtggtgctccatccatttcgtgttctatgccacaactgtacaggttcaaatgatagtagaacaactaattttgcatcggatactctctgcagcctctgttctgttcctactaattgcaggaccgagtctgtctttgtgatactatctgctttgcgtgcactgctcttttaggttgcagctgcaccttctattttgttcacctgagcattccattttggtcgactgaacaccgcaagtcctgaatatctttcttgaatcgtcggtcttacttggtgtgcactagttaaactgctgtgtatgcccttgcagctgccgttacagaaccagcgtccccgactccgaagatggaggagcattcgacctcgacgacgcctgtgtcggatgatcggtcgggatccgatgccttgtgtgcctgccgggtcttccttgtggccttggtcttgggagcagcagtcttgatcacgctgcagttctgatggatgttcgacctcgctcttgaaaggtctttgccctcgctcttgatacaatacttgtgcgcgatgctgcgccattcttgattggacctcttgttgcatacggtgaggtgccacgtgatgcagggcatgatattggtatacctgggaactcttcctgaccagtgcagtttgttccgtgcgcgttttgatgacctgacaacgcaagtagtaactgttatccggtgacataatcgtatcatttcaaacatggtacagatttatacaatgcagtttagtttctcttgcgatttattactttctgtgaatctgcatgccattctcttgcactcgctgagagagacggagaactcactggaatttgttttcactatacaaatatgcgcagttgcagcacacgcacgaacaaatacaaagtcacacatcattcgcatttttctttcagagttacacatttgaaatgaaggggaagaaaaacagaagtagcatcgtcagcggaatgaagcattacatgccaattggttgctgatcaggttcagtctgtggactgaatgtattctaggtacattatatgagcaatgacagtagccctgctgccacgatcgaggaggcgagaactacggaggatatcccacgacagagcttccggtgggcgctggacgggttgaacatgctgtcacgcgggtacatctgccccggcgtccccactatgccaaacaccggcgccggctgcacgtacgggtacacgtacggctcagccggcggtggaaggtagtacacgggcgttggttccggctccgacggcggtgggtagtagtagtacactgg

SEQ ID NO: 199 - DNA, 1047 bp, Triticum aestivum
  source 1..1047: mol_type = genomic DNA
atggcccccaccgccctcctcctcgtggtcctcgccgtcgccgccctccacgcccctgccgcctccgccgcgttgtcccaggagccaccagcgatgcgggatttttcttttgtggcccagacttacttctgcatttttgctgcgatttgtcatgattaccgttcagttaagcagtttgttgggttgcttgtttgatagtagtaatttcacaaaatattggcgatatttataaatagctaagggcttcgttacttggtggtttctctcaaagagttcacaaaagtcaatcattaaatcaattaagggggcaagtattagtagtactggctaactccatttgccatttgccaattgaaacagagctcaagtcactgcctgagatgcattttacacctccatcgccacctcccgcaccaaaactttctccaggtaaacgttctccctcttgtctaatgatttgataaatctgtgcccagtctattcgggtttcggttgaataaaccaattggcatttgggcagcatgttatatggtctctcctcatcccaaataaaacttgaacttgttttatcttgccgtgctccgtccattttgtgctctatgccgcaattgcacaggttcaatgatagtagaacacctagttttgcatcgcatactctctgcagcctctgttctgttcctgctaattgcagtatcgggtctgtctttgtgatactatctcctttgcatgcactgcacttttaggttgcagttgcacgtttgattctagtttgttcacctgagcattccattttggtcgactgaacaccgcaagtcctgaatatctttcttgaatcgtcggtcgtacttcgtgtgcactagttaaactgctgtctatgcccttgcagctgccgttccagaaccagcgtccccgaagatggaggagcattcgacctcagcgacgcctgtgccggatgatcggtcgggatccgatgccttgtgtgcctgccgggtcttccttgtggccttggtcttgggagcagcagtcttgatcacgctgcagttctga

SEQ ID NO: 200 - DNA, 735 bp, Triticum aestivum
  source 1..735: mol_type = genomic DNA
ttataaattcttgcatgtcaaactaaatcgatacattttaaaggcctaaagctgatctacaatattgttaaatatttgctttactaattcctgctctaacacaaaggttaatgacagaattaacaacatcgtgcatgtcaagaaaataacatcttgcatgcagcagcagtggccggctaaaagcgcatgcagtagcagtaccaatcagcaacagtggccggctaaaagcgcatgcagcagtagcaatcaacgtagtagtagagcagcagggccatgaacagggctgccgccagcccgcgaggcggccgttccccaggtacagtactgccgccacgccccttggcggctgggccccacacgccgctgccgttccgtcgccagcgccgattcccgttggtacagtgctgtcgcctcgctgtgtggcggcaggtgccacgtgtcgcctgccgcacctgccgccacagctgagggggtcctttttgttaaatttatcggcaggtggtcctttttgtcaattcattcgcgcatgtggtccttttcaataaaaaatctcctatgcgcccacctttgcgcggtcttgttaaacccctcagtattacactttttttttgcggggacagtattacacactcacgccctaaaaccaccaggcatcatcacctcaccacccatcccgccccaatacgagagaaagagagagagagagtggggacgggtgtgctgagagcctgagac

SEQ ID NO: 201 - DNA, 420 bp, Triticum aestivum
  source 1..420: mol_type = genomic DNA
tggatgttcgacctcgctcttgatacaatacctgtgcattgctgcgtcattcttgattggatcctcttgttgcatacggtgaagtgccacgtgatgcagggcgtgatacctgagaactcttcctgactagtgcagtttgttctgtgcgcgttttggatgacctgacaacgcaagtaactgttatccggtgacatactcgtatcgtttcaaacatggtatagatttatacaatgcagtctagtttctcttgcgattttctttctgtgaatctgcatgccatttccttgcgctcgctgagagagacgagaactcaggagcacagcacaggagcaaactctcatgctttcatggtgcatgaccttgctgcgctagctgcgccggtgaccactccggtcccggcggccaccagtcccccccccc

SEQ ID NO: 202 - DNA, 1356 bp, Triticum aestivum
  source 1..1356: mol_type = genomic DNA
gaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttagccgaaccccaaaaagagaaaaagtgatccctggtggtccggatttggcattgccgactgcccaaacatgcctcgccattttcattctccccttgcatatatgttcgatttgaggttcgcatatagtctagtcatatgcggactatttctgaggtttggttggacgacgtttctcctctctcctattcatgatgccttatgggcaagatatttgtttcactgatattttagttttccccgtttcttaaaacatgcttattctctttagcatggccaacaaatttgtaagagacccaccgagaggtgaccgaccaaaatagacaactaagcatgaaaaaatctcaccggcagaaggaaccgcgcggcgcaagcgcgggtatccctctagtacactactacttgcccatagagccagtggtgtacgagtacgaccgacgatgccttagacttagacttcccagtccgtgcaccggaagcacagaaccgtccacgactccacggacagaattcgctccatcaacacccaccaccacaccagcagtagctttcttcacgtgaaaaacaaccacatatacatgcctgccgtagacgacacccatgtactctaccatatatgtacggtcgagacgatacatcggcacatcgcgcctacgtgcccccaaagcatgaaatcaatcgacatgacacatgaacctcacgtgagcacacctctttttgattttatcttttttgcaaatcctcttttttgatttccggctcggctcctctagttgggcccgaagcagggcatatggcaaatgctccagctcgattccttaagtgggccggctgaggaggatcaagtatacacgaggatacagcccacttggccgtttggtggcatcccgcggtcctggtgcttgctgaacgcagcgaacgaccctggtttagtcccacctcgctcaccggaagaagcatcgatcggtttataagccccgcgcttgcaccctcgct

SEQ ID NO: 203 - DNA, 978 bp, synthetic construct
  misc_feature 1..978: note = synthesized
  source 1..978: mol_type = other DNA
atgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtaaacaacatcgtctctgtctttctactcattttctagctgacggacggaagtcaataacatcaactgtgtagaactaaattctagcctgttaatgtatcaaaaatgtcaatcttatttgcaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtcccgccccgtccggtcctgcccgtcaccgagatttgatctgtacattgctttgggtttccttctccattatctttcttgtttgttcctaagaatatgtgtatgtccatgttcatgtaccaacatgctcgagaaagcatgctcgtatgtgaatgcaatcggtggctatgtatatgcacatgcacataatatattatacaatctgtcgactatgtattcgggtaattaataatgagaaacatctttggcaccgcacgcacatcttttattccgccgctagacatagtttatgttggaatgatgagcaatcaagaataacacggtaaggcattgtctgatac

SEQ ID NO: 204 - DNA, 107 bp, synthetic construct
  misc_feature 1..107: note = synthesized
  source 1..107: mol_type = other DNA
gctgtcttcaacacaagaagacacgttttagagctagaaatagcaagttaaaataaggctagtccgttatcaacttgaaaaagtggcaccgagtcggtgcttttttt

SEQ ID NO: 205 - DNA, 1991 bp, Zea mays
  source 1..1991: mol_type = genomic DNA
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 206 - DNA, 4750 bp, synthetic construct
  misc_feature 1..4750: note = synthesized
  source 1..4750: mol_type = other DNA
atggctcctaagaagaagcggaaggttggtattcacggggtgcctgcggctatggataagaagtacagcattggtctggacatcgggacgaattccgttggctgggccgtgatcaccgatgagtacaaggtcccttccaagaagtttaaggttctggggaacaccgatcggcacagcatcaagaagaatctcattggagccctcctgttcgactcaggcgagaccgccgaagcaacaaggctcaagagaaccgcaaggagacggtatacaagaaggaagaataggatctgctacctgcaggagattttcagcaacgaaatggcgaaggtggacgattcgttctttcatagattggaagaaagtttcctcgtcgaggaagataagaagcacgagaggcatcctatctttggcaacattgtcgacgaggttgcctatcacgaaaagtaccccacaatctatcatctgcggaagaagcttgtggactcgactgataaggcggaccttagattgatctacctcgctctggcacacatgattaagttcaggggccattttctgatcgagggggatcttaacccggacaatagcgatgtggacaagttgttcatccagctcgtccaaacctacaatcagctctttgaggaaaacccaattaatgcttcaggcgtcgacgccaaggcgatcctgtctgcacgcctttcaaagtctcgccggcttgagaacttgatcgctcaactcccgggcgaaaagaagaacggcttgttcgggaatctcattgcactttcgttggggctcacaccaaacttcaagagtaattttgatctcgctgaggacgcaaagctgcagctttccaaggacacttatgacgatgacctggataaccttttggcccaaatcggcgatcagtacgcggacttgttcctcgccgcgaagaatttgtcggacgcgatcctcctgagtgatattctccgcgtgaacaccgagattacaaaggccccgctctcggcgagtatgatcaagcgctatgacgagcaccatcaggatctgacccttttgaaggctttggtccggcagcaactcccagagaagtacaaggaaatcttctttgatcaatccaagaacggctacgctggttatattgacggcggggcatcgcaggaggaattctacaagtttatcaagccaattctggagaagatggatggcacagaggaactcctggtgaagctcaatagggaggaccttttgcggaagcaaagaactttcgataacggcagcatccctcaccagattcatctcggggagctgcacgccatcctgagaaggcaggaagacttctacccctttcttaaggataaccgggagaagatcgaaaagattctgacgttcagaattccgtactatgtcggaccactcgcccggggtaattccagatttgcgtggatgaccagaaagagcgaggaaaccatcacaccttggaacttcgaggaagtggtcgataagggcgcttccgcacagagcttcattgagcgcatgacaaattttgacaagaacctgcctaatgagaaggtccttcccaagcattccctcctgtacgagtatttcactgtttataacgaactcacgaaggtgaagtatgtgaccgagggaatgcgcaagcccgccttcctgagcggcgagcaaaagaaggcgatcgtggaccttttgtttaagaccaatcggaaggtcacagttaagcagctcaaggaggactacttcaagaagattgaatgcttcgattccgttgagatcagcggcgtggaagacaggtttaacgcctcactggggacttaccacgatctcctgaagatcattaaggataaggacttcttggacaacgaggaaaatgaggatatcctcgaagacattgtcctgactcttacgttgtttgaggatagggaaatgatcgaggaacgcttgaagacgtatgcccatctcttcgatgacaaggttatgaagcagctcaagagaagaagatacaccggatggggaaggctgtcccgcaagcttatcaatggcattagagacaagcaatcagggaagacaatccttgactttttgaagtctgatggcttcgcgaacaggaattttatgcagctgattcacgatgactcacttactttcaaggaggatatccagaaggctcaagtgtcgggacaaggtgacagtctgcacgagcatatcgccaaccttgcgggatctcctgcaatcaagaagggtattctgcagacagtcaaggttgtggatgagcttgtgaaggtcatgggacggcataagcccgagaacatcgttattgagatggccagagaaaatcagaccacacaaaagggtcagaagaactcgagggagcgcatgaagcgcatcgaggaaggcattaaggagctggggagtcagatccttaaggagcacccggtggaaaacacgcagttgcaaaatgagaagctctatctgtactatctgcaaaatggcagggatatgtatgtggaccaggagttggatattaaccgcctctcggattacgacgtcgatcatatcgttcctcagtccttccttaaggatgacagcattgacaataaggttctcaccaggtccgacaagaaccgcgggaagtccgataatgtgcccagcgaggaagtcgttaagaagatgaagaactactggaggcaacttttgaatgccaagttgatcacacagaggaagtttgataacctcactaaggccgagcgcggaggtctcagcgaactggacaaggcgggcttcattaagcggcaactggttgagactagacagatcacgaagcacgtggcgcagattctcgattcacgcatgaacacgaagtacgatgagaatgacaagctgatccgggaagtgaaggtcatcaccttgaagtcaaagctcgtttctgacttcaggaaggatttccaattttataaggtgcgcgagatcaacaattatcaccatgctcatgacgcatacctcaacgctgtggtcggaacagcattgattaagaagtacccgaagctcgagtccgaattcgtgtacggtgactataaggtttacgatgtgcgcaagatgatcgccaagtcagagcaggaaattggcaaggccactgcgaagtatttcttttactctaacattatgaatttctttaagactgagatcacgctggctaatggcgaaatccggaagagaccacttattgagaccaacggcgagacaggggaaatcgtgtgggacaaggggagggatttcgccacagtccgcaaggttctctctatgcctcaagtgaatattgtcaagaagactgaagtccagacgggcgggttctcaaaggaatctattctgcccaagcggaactcggataagcttatcgccagaaagaaggactgggatccgaagaagtatggaggtttcgactcaccaacggtggcttactctgtcctggttgtggcaaaggtggagaagggaaagtcaaagaagctcaagtctgtcaaggagctcctgggtatcaccattatggagaggtccagcttcgaaaagaatccgatcgattttctcgaggcgaagggatataaggaagtgaagaaggacctgatcattaagcttccaaagtacagtcttttcgagttggaaaacggcaggaagcgcatgttggcttccgcaggagagctccagaagggtaacgagcttgctttgccgtccaagtatgtgaacttcctctatctggcatcccactacgagaagctcaagggcagcccagaggataacgaacagaagcaactgtttgtggagcaacacaagcattatcttgacgagatcattgaacagatttcggagttcagtaagcgcgtcatcctcgccgacgcgaatttggataaggttctctcagcctacaacaagcaccgggacaagcctatcagagagcaggcggaaaatatcattcatctcttcaccctgacaaaccttggggctcccgctgcattcaagtattttgacactacgattgatcggaagagatacacttctacgaaggaggtgctggatgcaacccttatccaccaatcgattactggcctctacgagacgcggatcgacttgagtcagctcggtggcgataagagacccgcagcaaccaagaaggcagggcaagcaaagaagaagaagtgactaactaactaggcgcgcccgggtaccgtttttctcagacagttttctaaaaaaagggcgtttctggggaagttcgagatggttcgtaaggtgttactggctcctgtgaaccaatacatgatactgccatgataagggttataattagtcaagcagagtaagaagaaacaacagtagcagtgactccgattcctgaagatgagtcatatttgtcttgtgctcctgctgtatgaaatggatcgcatgtgtatattcgtcgccgcgccgcactggtgtaacctgttgcctcagagtttgcttttagctggttctgttttaaaaataagtactgttttttggttggctgcaagccattctgaacttcagtttaccaattgtttttatgttgtggttgaatattttaattttttatttaatgtttggttctttttttatatatatttgcaaaaatgatacaagtggtcaagttttcatatagtatgggctctatttcctagagctctacctctaggaacgaattttgtggaggttttcttttggctag

SEQ ID NO: 207 - DNA, 517 bp, synthetic construct
  misc_feature 1..517: note = synthesized
  source 1..517: mol_type = other DNA
gtttttctcagacagttttctaaaaaaagggcgtttctggggaagttcgagatggttcgtaaggtgttactggctcctgtgaaccaatacatgatactgccatgataagggttataattagtcaagcagagtaagaagaaacaacagtagcagtgactccgattcctgaagatgagtcatatttgtcttgtgctcctgctgtatgaaatggatcgcatgtgtatattcgtcgccgcgccgcactggtgtaacctgttgcctcagagtttgcttttagctggttctgttttaaaaataagtactgttttttggttggctgcaagccattctgaacttcagtttaccaattgtttttatgttgtggttgaatattttaattttttatttaatgtttggttctttttttatatatatttgcaaaaatgatacaagtggtcaagttttcatatagtatgggctctatttcctagagctctacctctaggaacgaattttgtggaggttttcttttggctag

SEQ ID NO: 208 - DNA, 486 bp, synthetic construct
  misc_feature 1..486: note = synthesized
  source 1..486: mol_type = other DNA
ggaattcccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactaagct

SEQ ID NO: 209 - DNA, 856 bp, synthetic construct
  misc_feature 1..856: note = synthesized
  source 1..856: mol_type = other DNA
ctctagaactagtggatctcgatgtgtagtctacgagaagggttaaccgtctcttcgtgagaataaccgtggcctaaaaataagccgatgaggataaataaaatgtggtggtacagtacttcaagaggtttactcatcaagaggatgcttttccgatgagctctagtagtacatcggacctcacatacctccattgtggtgaaatattttgtgctcatttagtgatgggtaaattttgtttatgtcactctaggttttgacatttcagttttgccactcttaggttttgacaaataatttccattccgcggcaaaagcaaaacaattttattttacttttaccactcttagctttcacaatgtatcacaaatgccactctagaaattctgtttatgccacagaatgtgaaaaaaaacactcacttatttgaagccaaggtgttcatggcatggaaatgtgacataaagtaacgttcgtgtataagaaaaaattgtactcctcgtaacaagagacggaaacatcatgagacaatcgcgtttggaaggctttgcatcacctttggatgatgcgcatgaatggagtcgtctgcttgctagccttcgcctaccgcccactgagtccgggcggcaactaccatcggcgaacgacccagctgacctctaccgaccggacttgaatgcgctaccttcgtcagcgacgatggccgcgtacgctggcgacgtgcccccgcatgcatggcggcacatggcgagctcagaccgtgcgtggctggctacaaatacgtaccccgtgagtgccctagctagaaacttacacctgcaactgcgagagcgagcgtgtgagtgtagccgagta

SEQ ID NO: 210 - DNA, 678 bp, synthetic construct
  misc_feature 1..678: note = synthesized
  source 1..678: mol_type = other DNA
atggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtag

SEQ ID NO: 211 - DNA, 311 bp, synthetic construct
  misc_feature 1..311: note = synthesized
  source 1..311: mol_type = other DNA
cctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgc

SEQ ID NO: 212 - DNA, 1502 bp, synthetic construct
  misc_feature 1..1502: note = synthesized
  source 1..1502: mol_type = other DNA
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag